SEQUENCE LISTING (12 sequences)

SEQ ID NO: 1 - skipped

SEQ ID NO: 2 - protein, 14 aa, synthetic construct
  REGION 1..14: note = Synthetic peptide
KDDYAGGGAAGKDA

SEQ ID NO: 3 - skipped

SEQ ID NO: 4 - protein, 7 aa, synthetic construct
  REGION 1..7: note = Synthetic peptide
ENLYFQG

SEQ ID NO: 5 - protein, 7 aa, synthetic construct
  REGION 1..7: note = Synthetic peptide
ENLYFQS

SEQ ID NO: 6 - protein, 4 aa, synthetic construct
  REGION 1..4: note = Synthetic peptide
KAKA

SEQ ID NO: 7 - protein, 4 aa, synthetic construct
  REGION 1..4: note = Synthetic peptide
KAAK

SEQ ID NO: 8 - protein, 5 aa, synthetic construct
  REGION 1..5: note = Synthetic peptide
DFSKL

SEQ ID NO: 9 - protein, 9 aa, synthetic construct
  REGION 1..9: note = Synthetic peptide
KAKADFSKL

SEQ ID NO: 10 - protein, 9 aa, synthetic construct
  REGION 1..9: note = Synthetic peptide
KAAKDFSKL

SEQ ID NO: 11 - protein, 6 aa, synthetic construct
  REGION 1..6: note = Synthetic peptide
MEPAER

SEQ ID NO: 12 - protein, 9 aa, synthetic construct
  REGION 1..9: note = Synthetic peptide
MLKPALEPR

(19) United States
(12) Patent Application Publication
(10) Pub. No.: US 2024/0201198 A1
(43) Pub. Date: Jun. 20, 2024

(54) COMPOSITIONS, METHODS, AND UTILITY OF CONJUGATED BIOMOLECULE BARCODES

(71) Applicants: BOARD OF REGENTS, THE UNIVERSITY OF TEXAS SYSTEM, Austin, TX (US); ERISYON, INC., Austin, TX (US)

(72) Inventors: Edward MARCOTTE, Austin, TX (US); Eric V. ANSLYN, Austin, TX (US); Jagannath SWAMINATHAN, Austin, TX (US); Cecil J. HOWARD, II, Austin, TX (US); Angela M. BARDO, Austin, TX (US); Zachary Booth SIMPSON, Austin, TX (US)

(21) Appl. No.: 18/518,854

(22) Filed: Nov. 24, 2023

Related U.S. Application Data
(63) Continuation of application No. PCT/US2022/031079, filed on May 26, 2022.
(60) Provisional application No. 63/193,436, filed on May 26, 2021.

(51) Int. Cl. G01N 33/68

(52) U.S. Cl. CPC G01N 33/6824; G01N 33/6821; G01N 33/6848; G01N 2333/9015; G01N 2458/15

(57) ABSTRACT

The present disclosure provides a range of methods for dense information storage in oligomeric constructs, such as peptides. Various methods of the present disclosure provide for information storage in oligomers and oligomer libraries, and information retrieval by subsequent oligomer sequencing or analysis. The present disclosure further provides methods for appending information to materials and molecules with analyzable oligomeric constructs.

### CROSS-REFERENCE

This application is a continuation of International Application No. PCT/US2022/031079, filed May 26, 2022, which claims the benefit of priority to U.S. Provisional Patent Application No. 63/193,436, filed on May 26, 2021, which are hereby incorporated by reference in their entirety.

### STATEMENT REGARDING FEDERALLY SPONSORED RESEARCH OR DEVELOPMENT

This invention was made with government support under Grant No. R35 GM122480 awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Nov. 21, 2023, is named UTSBP1255US.xml and is 13,830 bytes in size.

### BACKGROUND

Bioanalytical methods typically capture a sparse fraction of the information contained within a system. For example, a simple bacterial cell often comprises millions of proteins dynamically progressing through ranges of conformations, activities, cofactor associations, oxidation states, and/or sub-cellular or extracellular localizations. Biological and/or diagnostic assays typically discern a fraction of this complexity, focusing instead on singular aspects of dynamic and/or complex biological systems, or destroying a system in the process of analysis. In many cases, the limited information obtained from such assays is insufficient for identifying health (e.g., disease) or system-level markers. Accordingly, there is a need for enhanced information tracking and/or retrieval from complex and/or active biological systems.

### SUMMARY

The present disclosure provides a range of compositions, systems, and/or methods for ascertaining information from complex systems. Aspects of the present disclosure provide methods for appending information to individual species in the form of information dense oligomeric barcodes. Such a method may comprise coupling an oligomeric barcode, such as an oligopeptide, to a molecule or a species of interest, thereby storing information of the oligomeric barcode within The biomolecule or species. The present disclosure also provides methods for retrieving information from oligomeric barcodes, including collecting oligomeric barcodes from systems (e.g., cleaving and/or separating a plurality oligomeric barcodes from a complex system), storing oligomeric barcodes (e.g., lyophilized coupled to a surface), and/or extracting information from the oligomeric barcodes (e.g., by fluorosequencing or nanopore sequencing the oligomeric barcodes). The present disclosure further provides methods for selectively coupling oligomeric barcodes to individual species of interest within complex samples.

Various aspects of the present disclosure provide a method for identifying a biomolecule, the method comprising: (a) providing the biomolecule having coupled thereto an oligomeric barcode, wherein the oligomeric barcode comprises a plurality of monomeric subunits, wherein at least a subset of the monomeric subunits comprise a label; and/or (b) identifying the label, wherein the identifying is by sequencing by degradation. In some embodiments, the biomolecule is a polypeptide. In some embodiments, the biomolecule is a protein. In some embodiments, the method further comprises coupling the oligomeric barcode to the biomolecule.

In some embodiments, the coupling comprises enzymatic ligation. In some embodiments, the coupling comprises transesterification. In some embodiments, the coupling comprises chemical coupling or enzymatic coupling. In some embodiments, the coupling comprises expressing the biomolecule coupled to the oligomeric barcode or co-translation of the oligomeric barcode as a peptide tag. In some embodiments, the coupling comprises expressing the biomolecule coupled to the oligomeric barcode. In some embodiments, the coupling comprises chemically synthesizing the biomolecule having coupled thereto the oligomeric barcode.

In some embodiments, the oligomeric barcode comprises a polymer. In some embodiments, the oligomeric barcode comprises a polypeptide. In some embodiments, the oligomeric barcode comprises from about 2 to about 30 amino acids. In some embodiments, the oligomeric barcode comprises a non-natural amino acid. In some embodiments, the plurality of monomeric subunits is a plurality of amino acids. In some embodiments, the oligomeric barcode comprises at least about 2, at least about 5, at least about 10, at least about 15, at least about 20, at least about 25, or at least about 30 amino acids. In some embodiments, the oligomeric barcode comprises about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids. In some embodiments, the oligomeric barcode comprises at most about 2, at most about 5, at most about 10, at most about 15, at most about 20, at most about 25, or at most about 30 amino acids.

In some embodiments, the label is coupled to an internal monomeric subunit of the plurality of monomeric subunits. In some embodiments, the label is an amino acid specific label. In some embodiments, the amino acid specific label comprises a methionine specific label, an arginine specific label, a histidine specific label, a tyrosine specific label, a carboxylic acid R-group specific label, a lysine specific label, a cysteine specific label, a tryptophan specific label, or any combination thereof. In some embodiments, the amino acid specific label comprises a non-natural amino acid specific label. In some embodiments, the non-natural amino acid specific label is a phosphoserine specific label, phosphothreonine specific label, pyroglutamic acid specific label, hydroxyproline specific label, azidolysine specific label, or dehydroalanine specific label. In some embodiments, the label is a fluorescent label. In some embodiments, the label is a dye.

In some embodiments, the sequencing by degradation comprises Edman degradation. In some embodiments, the sequencing by degradation comprises subjecting the oligomeric barcode to conditions sufficient to remove at least one monomeric subunit from the oligomeric barcode. In some embodiments, the sequencing by degradation comprises subjecting the oligomeric barcode to conditions sufficient to remove at least one amino acid from the oligomeric barcode. In some embodiments, the label generates at least one signal or at least one signal change. In some embodiments, the at least one signal or the at least one signal change is an optical signal. In some embodiments, the at least one signal or the at least one signal change comprises a plurality of signals of different intensities. In some embodiments, the at least one signal or the at least one signal change comprises a plurality of signals of different frequencies or signals of different frequency ranges.

In some embodiments, the sequencing by degradation comprises enzymatic cleavage of the oligomeric barcode from the biomolecule. In some embodiments, the sequencing by degradation comprises chemical cleavage of the oligomeric barcode from the biomolecule. In some embodiments, the chemical cleavage comprises cyanogen bromide cleavage, BNPS-skatole cleavage, formic acid cleavage, hydroxylamine cleavage, 2-nitro-5-thiocyanobenzoic acid cleavage, or any combination thereof. In some embodiments, the oligomeric barcode is coupled to the biomolecule via an N-terminal tag, a C-terminal tag, or an amino acid sidechain. In some embodiments, the N-terminal tag is a purification tag, a localization signal, a fluorescent tag, a chemically modifiable tag, or an enzymatically modifiable tag. In some embodiments, the C-terminal tag is a purification tag, a localization signal, a fluorescent tag, a chemically modifiable tag, or an enzymatically modifiable tag.

In some embodiments, the oligomeric barcode is coupled to the biomolecule via a cleavable linker. In some embodiments, the cleavable linker comprises a TEV protease cleavage site, a thrombin cleavage site, an enterokinase cleavage site, or any combination thereof. In some embodiments, the cleavable linker comprises an amino acid cleavage sequence not present in the oligomeric barcode. In some embodiments, the cleavable linker comprises a chemically cleavable group. In some embodiments, the chemically cleavable comprises a disulfide. In some embodiments, wherein the method further comprises cleaving the oligomeric barcode from the biomolecule.

In some embodiments, wherein the method further comprises separating the oligomeric barcode from the biomolecule after the cleaving. In some embodiments, wherein the separating comprises isoelectric focusing. In some embodiments, wherein the separating comprises chromatographic separation. In some embodiments, wherein the separating comprises electrophoretic separation.

In some embodiments, wherein the method further comprises coupling the oligomeric barcode to a substrate after the cleaving. In some embodiments, wherein the method further comprises coupling the oligomeric barcode to a substrate after the separating. In some embodiments, wherein the oligomeric barcode is selected from a library comprising at least 216 uniquely identifiable oligomeric barcodes. In some embodiments, wherein the identifying comprises a resolution capable of resolving a single oligomeric barcode. In some embodiments, wherein the biomolecule and the oligomeric barcode comprise a common sequence.

Various aspects of the present disclosure provide a method comprising: (a) providing a polypeptide immobilized to a support, wherein the polypeptide comprises at least one labeled internal amino acid, and wherein the polypeptide encodes data; (b) detecting at least one signal or signal change from the polypeptide immobilized to the support to identify at least a portion of a sequence of the polypeptide; and/or (c) subjecting the polypeptide to conditions sufficient to remove at least one amino acid from the polypeptide.

In some embodiments, the at least one labeled internal amino acid comprises a plurality of amino acid specific labels. In some embodiments, the amino acid specific labels comprise a methionine specific label, an arginine specific label, a histidine specific label, a tyrosine specific label, a carboxylic acid-containing amino acid specific label, a lysine specific label, a cysteine specific label, or any combination thereof. In some embodiments, the at least one labeled internal amino acid comprises an optically detectable label. In some embodiments, the at least one amino acid is removed from an N-terminus of the polypeptide.

In some embodiments, subsequent to (c), the at least one labeled internal amino acid becomes a labeled terminal amino acid. In some embodiments, the at least one labeled internal amino acid is from a plurality of labeled amino acids, and/or wherein the at least one signal or signal change comprises a collective signal from the plurality of labeled amino acids. In some embodiments, the plurality of labeled amino acids comprise amino acids with different labels. In some embodiments, the different labels generate signals with different signal patterns. In some embodiments, the at least one labeled internal amino acid comprises one or more members selected from the group consisting of lysine, glutamate, and aspartate. In some embodiments, the at least one labeled internal amino acid comprises an amino acid having a dye coupled thereto, which dye generates the at least one signal or signal change. In some embodiments, the at least one signal or signal change is an optical signal. In some embodiments, the at least one signal or signal change comprises a plurality of signals of different intensities. In some embodiments, the at least one signal or signal change comprises a plurality of signals of different frequencies or frequency ranges.

In some embodiments, the method further comprises cleaving the polypeptide from the support. In some embodiments, at least one amino acid is removed from the polypeptide by a degradation reaction. In some embodiments, the degradation reaction is Edman degradation. In some embodiments, the polypeptide is a protein. In some embodiments, the polypeptide is part of a protein. In some embodiments, the at least one signal or signal change is detected with an optical detector having single-molecule sensitivity. In some embodiments, the method further comprises processing the at least the portion of the sequence against a reference sequence to identify the polypeptide or a protein from which the polypeptide is derived. In some embodiments, the method further comprises, subsequent to (c), (i) identifying the at least the portion of the sequence of the polypeptide to identify the polypeptide, and/or (ii) using the polypeptide identified in (i) to quantify the polypeptide or a protein from which the polypeptide was derived.

In some embodiments, in (a), less than all amino acids of the polypeptide are labeled. In some embodiments, the method further comprises (i) repeating (b) and/or (c) to detect at least one additional signal or signal change from the polypeptide immobilized to the support and/or (ii) using the at least one signal or signal change and/or the at least one additional signal or signal change to identify the at least the portion of the sequence. In some embodiments, the detecting identifies a sequence of the polypeptide. In some embodiments, the detecting is performed at a read rate of at least 36 bits/s. In some embodiments, the detecting comprises fluorimetry. In some embodiments, the detecting comprises imaging.

In some embodiments, the method further comprises assigning the polypeptide a optically resolvable address. In some embodiments, the optically resolvable address comprises digital information. In some embodiments, the method further comprises comparing the portion of the sequence of the polypeptide against a database of known sequences. In some embodiments, the method further comprises, prior to (a), coupling the polypeptide to the support. In some embodiments, the method further comprises determining a physical property of the polypeptide. In some embodiments, the physical property is selected from the group consisting of isoelectric point, molecular weight, and hydrophobicity index. In some embodiments, the method further comprises, prior to (a), coupling the polypeptide to an array. In some embodiments, the method further comprises lyophilizing the array. In some embodiments, the array comprises an information storage density of at least 10⁷ bytes/cm³. In some embodiments, the array comprises an information storage density of at least 10³⁰ bytes/cm³.

Another aspect of the present disclosure provides a non-transitory computer readable medium comprising machine executable code that, upon execution by one or more computer processors, implements an of the methods above or elsewhere herein.

Another aspect of the present disclosure provides a system comprising one or more computer processors and/or computer memory coupled thereto. The computer memory comprises machine executable code that, upon execution by one or more computer processors, implements any of the methods above or elsewhere herein.

Additional aspects and/or advantages of the present disclosure will become readily apparent to those skilled in this art from the following detailed description, wherein only illustrative embodiments of the present disclosure are shown and/or described. As will be realized, the present disclosure is capable of other and/or different embodiments, and/or its several details are capable of modifications in various obvious respects, all without departing from the disclosure. Accordingly, the drawings and/or description are to be regarded as illustrative in nature, and/or not as restrictive.

### INCORPORATION BY REFERENCE

All publications, patents, and/or patent applications mentioned in this specification are herein incorporated by reference to the same extent as if each individual publication, patent, or patent application was specifically and/or individually indicated to be incorporated by reference. To the extent publications and/or patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and/or advantages of the present invention will be obtained by reference to the following detailed description that sets forth illustrative embodiments, in which the principles of the invention are utilized, and/or the accompanying drawings (also “figure” and “FIG.” herein), of which:

FIG. 1 shows a computer system that is programmed or otherwise configured to implement methods provided herein.

FIG. 2 provides an example of a plasmid encoding a peptide barcode adjacent to a gene of interest.

FIG. 3 illustrates a method for peptide-based information storage consistent with the present disclosure.

### DETAILED DESCRIPTION

While various embodiments of the invention have been shown and/or described herein, it will be obvious to those skilled in the art that such embodiments are provided by way of example only. Numerous variations, changes, and/or substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3, respectively.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1, respectively.

The term “biomolecule” as used herein generally refers to any biomolecule associated with a cell. In some examples, a biomolecule is polypeptide, peptide, or protein. In some embodiments, the biomolecule is a polypeptide. In some embodiments, the biomolecule is a protein. In some embodiments, the biomolecule is an antibody. In some embodiments, the biomolecule is an enzyme, a hormonal protein, a structural protein, a storage protein, or a transport protein.

The terms “polypeptide,” “oligopeptide,” and/or “peptide” generally refer to a polymer of amino acids in which an amino acid may be linked to another amino acid by a peptide bond. In some examples, a polypeptide is a protein. The amino acid may be a naturally occurring amino acid or a non-naturally occurring amino acid (i.e., amino acid analogue such as azidolysine). The polymer can be linear or branched and/or can include modified amino acids, and/or may be interrupted or terminated by non-amino acids. The polymer may comprise a non-amino acid building block, such as an ethylene glycol or functionalized alkyl moiety. Peptides can occur as single chains or associated chains. The polymer may include a plurality of amino acids and/or may have a secondary and/or tertiary structure (i.e., protein). In some examples, the polymer comprises at least 2, 3, 4, 5, 6, 7, 8, 9, 10, 20, 30, 40, 50, 100, 1,000, 10,000, or more amino acid. The polypeptide may be a fragment of a larger polymer. In some examples, the polypeptide is a fragment of a larger polypeptide, such as a fragment of a protein.

The term “amino acid,” as used herein, generally refers to a naturally occurring or non-naturally occurring amino acid (amino acid analogue). The non-naturally occurring amino acid may be a synthesized amino acid. In some embodiments, the non-natural amino acid is citrulline (Cit), hydroxyproline (Hyp), norleucine (Nle), 3-nitrotyrosine, nitroarginine, ornithine (Orn), naphtylalanine (Nal), Abu, DAB, methionine sulfoxide, or methionine sulfone. In some embodiments, the non-natural amino acid is an analogue of alanine, valine, glycine, and/or leucine. In some embodiments, the non-natural amino acid is an analogue of arginine and/or lysine. In some embodiments, the non-natural amino acid is racemic.

As used herein, the terms “amino acid sequence,” “peptide sequence,” “oligopeptide sequence,” and/or “polypeptide sequence,” as used herein, generally refer to at least two amino acids or amino acid analogs that are covalently linked by a peptide (amide) bond or an analog of a peptide bond. The term peptide includes oligomers and/or polymers of amino acids or amino acid analogs. The amino acids of the peptide may be L-amino acids or D-amino acids. A peptide, polypeptide, or protein may be synthetic, recombinant, or naturally occurring. A synthetic peptide may be a peptide that is produced by artificial approaches in vitro.

As used herein, the term “side chains” or “R” generally refers to unique structures attached to the alpha carbon (attaching the amine and/or carboxylic acid groups of the amino acid) that render uniqueness to each type of amino acid. R groups have a variety of shapes, sizes, charges, and/or reactivities, such as charged polar side chains, either positively or negatively charged, such as lysine (+), arginine (+), histidine (+), aspartate (−), and/or glutamate (−); amino acids can also be basic, such as lysine, or acidic, such as glutamic acid; uncharged polar side chains have hydroxyl, amide, or thiol groups, such as cysteine having a chemically reactive side chain, i.e., a thiol group that can form bonds with another cysteine, serine (Ser) and threonine (Thr), that have hydroxylic R side chains of different sizes; asparagine (Asn), glutamine (Gln), and/or tyrosine (Tyr); non-polar hydrophobic amino acid side chains include the amino acid glycine, alanine, valine, leucine, and/or isoleucine having aliphatic hydrocarbon side chains ranging in size from a methyl group for alanine to isomeric butyl groups for leucine and/or isoleucine; methionine (Met) has a thiol ether side chain; proline (Pro) has a cyclic pyrrolidine side group. Phenylalanine (Phe) and/or tryptophan (Trp) contain aromatic side chains, which are characterized by bulk as well as lack of polarity.

The term “cleavable unit,” as used herein, generally refers to a molecule that can be split into at least two molecules. Non-limiting examples of cleavage reagents and/or conditions to split a cleavable unit include: enzymes, nucleophilic or basic reagents, reducing agents, photo-irradiation, electrophilic or acidic reagents, organometallic or metal reagents, and/or oxidizing reagents.

The term “sample,” as used herein, generally refers to a sample containing or suspected of containing a polypeptide. For example, a sample can be a biological sample containing one or more polypeptides. The biological sample can be obtained (e.g., extracted or isolated) from or include blood (e.g., whole blood), plasma, serum, urine, saliva, mucosal excretions, sputum, stool and/or tears. The biological sample can be a fluid or tissue sample (e.g., skin sample). In some examples, the sample is obtained from a cell-free bodily fluid, such as whole blood, saliva, or urine. In some examples, the sample can include circulating tumor cells. In some examples, the sample is an environmental sample (e.g., soil, waste, ambient air), industrial sample (e.g., samples from any industrial processes), and/or food samples (e.g., dairy products, vegetable products, and/or meat products). The sample may be processed prior to loading into a microfluidic device. For example, the sample may be processed to purify the polypeptides and/or to include reagents.

As used herein, sequencing of peptides “at the single molecule level” generally refers to amino acid sequence information obtained from individual (i.e., single) peptide molecules in a mixture of diverse peptide molecules. The amino acid sequence information may be obtained from an entirety of an individual peptide molecule or one or more portion of the individual peptide molecule, such as a contiguous amino acid sequence of at least a portion of the individual peptide molecule. Alternatively, partial amino acid sequence information may be obtained, which may allow for identification of the peptide or protein. Partial amino acid sequence information, including for example, the pattern of a specific amino acid residue (i.e., lysine) within individual peptide molecules, may be sufficient to uniquely identify an individual peptide molecule. For example, a pattern of amino acids may comprise a plurality of identified positions (e.g., identified as a particular amino acid type, such as lysine, or identified as a particular set of amino acids, such as the set of carboxylate side chain-containing amino acids), and/or a plurality of unidentified positions. The sequence of identified positions may be searched against a known proteome of a given organism to identify the individual peptide molecule. The search may comprise a correction or tolerance for sequencing errors, such as misidentified positions, or misphasing due to unsuccessful or incomplete peptide cleavage steps. In some examples, sequencing of a peptide at the single molecule level may identify a pattern of a certain type of amino acid (e.g., lysine) in an individual peptide molecule. Such information may be used to identify a macromolecule (e.g., protein) from which the peptide was derived. This method may advantageously preclude the need to identify all amino acids of the peptide.

As used herein, the term “barcode” or “oligopeptide barcode” generally refers to a species that is coupled or provided coupled to a biomolecule. In some embodiments, a barcode comprises a polypeptide. In some embodiments, a barcode is a polypeptide sequence. The barcode may comprise information in the form of a sequence, a composition, a recognizable chemical feature (e.g., an optically detectable label), a physical property (e.g., isoelectric point), or any combination therein. In coupling the barcode to the species, the species may carry the information contained by the barcode. The information may be used to identify point of origin, such as the cell, volume, or emulsion from which a species was generated; a chemical treatment, such as a reaction or condition to which the species was subjected; an interaction, such as a transient binding event with a second species; or any combination thereof. Barcode information may be extracted by analyzing (e.g., sequencing or identifying an electrophoretic mobility of) the barcode. In this way, barcoding can increase the amount of information derived from an experiment or process.

As used herein, the term “Edman degradation” generally refers to methods comprising chemical removal of amino acids from peptides or proteins. In some cases, Edman degradation denotes terminal (e.g., N- or C-terminal) amino acid removal. In some cases, Edman degradation refers to N-terminal amino acid removal. In some cases, Edman degradation refers to N-terminal amino acid removal through isothiocyanate (e.g., phenyl isothiocyanate) coupling and/or cyclization with the terminal amine group of an N-terminal residue, such that the N-terminal amino acid is removed from a peptide. In some cases, Edman degradation broadly encompasses N-terminal amino acid functionalization leading to N-terminal amino acid removal. In some cases, Edman degradation encompasses C-terminal amino acid removal. In some cases, Edman degradation comprises terminal amino acid functionalization (e.g., N-terminal amino acid isothiocyanate functionalization) followed by enzymatic removal (e.g., by an ‘Edmanase’ with specificity for chemically derivatized N-terminal amino acids).

As used herein, the term “single molecule sensitivity” generally refers to the ability to acquire data (including, for example, amino acid sequence information) from individual molecules (e.g., individual peptide molecules) from mixtures of molecules. Single molecule sensitivity may include the ability to simultaneously record the fluorescence intensity of multiple individual (i.e., single) molecules distributed across a surface (including, for example, a glass slide, or a glass slide whose surface has been chemically modified). Certain commercial optical devices can be applied in this manner. For example, a conventional microscope equipped with total internal reflection illumination and/or an intensified charge-couple device (CCD) detector is available. Imaging with a high sensitivity CCD camera allows the instrument to simultaneously record the fluorescent intensity of multiple individual (i.e., single) peptide molecules distributed across a surface. Image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

As used herein, “single molecule resolution” refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). In one embodiment, this may include the ability to simultaneously record the fluorescent intensity of multiple individual (i.e. single) peptide molecules distributed across the glass surface. Optical devices are commercially available that can be applied in this manner. For example, a conventional microscope equipped with total internal reflection illumination and/or an intensified charge-couple device (CCD) detector is available. Imaging with a high sensitivity CCD camera allows the instrument to simultaneously record the fluorescent intensity of multiple individual (i.e. single) peptide molecules distributed across a surface. In one embodiment, image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

As used herein, the term “collective signal” refers to the combined signal that results from the first and/or second labels attached to an individual peptide molecule. As used herein, the term “experimental cycle” refers to one round of single molecule sequencing, comprised of the Edman degradation of a single amino acid residue followed by TIRF measurement of fluorescence intensities.

As used herein, the term “support” generally refers to an entity to which a substance (e.g., molecular construct) can be immobilized. The solid may be a solid or semi-solid (e.g., gel) support. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, or a functionalized surface. The support may be planar. As an alternative, the support may be non-planar, such as including one or more wells. A bead can be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional group that can react with a molecule (e.g., an aldehyde, a phosphate, a silicate, an ester, an acid, an amide, an alkyne, an azide, or an aldehyde dithiolane). The functional group may couple specifically to an N-terminus or a C-terminus of a peptide. The functional group may couple specifically to an amino acid side chain. The functional group may couple to a side chain of an amino acid (e.g., the acid of a glutamate or aspartate, the thiol of a cysteine, the amine of a lysine, or the amide of a glutamine or asparagine). The functional group may couple specifically to a reactive group on a particular species, such as a label. In some examples of functionalized beads, the functional motif can be reversibly coupled and/or cleaved. A functional motif can also irreversibly couple to a molecule.

As used herein, the term “array” generally refers to a population of species or sites. Such populations of sites can often be differentiated from one another according to relative location. For example, a plurality of molecules coupled to a plurality of sites of an array may be differentiated from each other by ascertaining their locations with an imaging technique. A location may denote a 1-dimensional (e.g., location along a channel), 2-dimensional (e.g., location on a surface), or 3-dimensional (e.g., location within a gel or polymer matrix) address. An individual site of an array can include one or more molecules of a particular type. For example, a site can include a single polypeptide having a particular sequence or a site can include several polypeptides sharing a sequence or comprising a plurality of different sequences. A plurality of sites of an array can comprise a plurality of features of a substrate. Such features may include, without limitation, wells in a substrate, chambers in a substrate, beads (or other particles) in or on a substrate, projections from a substrate, ridges on a substrate, or channels in a substrate. A plurality of sites of an array may be disposed on a plurality of substrates. Such different molecules may have the same or different sequences. An array may include one or more wells, and/or a well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that can couple to a reactive group, such as an amino acid side chain, C-terminal carboxylate, or N-terminal amine. The label may comprise at least one reactive group (e.g., a first reactive group and/or a second reactive group). The at least one reactive group may be configured to couple to a polypeptide. The at least one reactive group may be configured to couple to a support. The at least one reactive group may be coupled to or configured to couple to a detectable moiety. A label may provide a measurable signal.

As used herein, the term “polymer matrix” generally refers to a continuous phase material that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and/or crosslinked polymer units. A polymer matrix may also contain non-polymeric species intercalated within its interstitial spaces not occupied by polymer chains. The intercalated species may be solid, liquid, or gaseous species. For example, the term “polymer matrix” may encompass desiccated hydrogels, hydrated hydrogels, and/or hydrogels containing glass fibers.

The present disclosure provides a system that can employ the use of polypeptide molecules for data storage. The system can include a solid state substrate with locations on the substrate for containing biological and/or chemical matter. The locations on the substrate may be referred to as “pixels” and/or each individual pixel is arranged such that the substrate has an array of pixels.

The term “polymerase,’ as used herein, generally refers to any enzyme capable of catalyzing a polymerization reaction. Examples of polymerases include, without limitation, a nucleic acid polymerase. A polymerase can be a polymerization enzyme. In some cases, a transcriptase or a ligase is used (i.e., enzymes which catalyze the formation of a bond).

Peptide sequence information may be obtained from a polypeptide molecule or from one or more portions of the polypeptide molecule. Peptide sequencing may provide complete or partial amino acid sequence information for a peptide sequence or a portion of a peptide sequence. At least a portion of the peptide sequence may be determined at the single molecule level. In some cases, partial amino acid sequence information, including for example, the relative positions of a specific type of amino acid (e.g., lysine) within a peptide or portion of a peptide, may be sufficient to uniquely identify an individual peptide molecule. For example, a pattern of amino acids, such as, for example, X-X-X-Lys-X-X-X-X-Lys-X-Lys (SEQ ID NO: 1), which indicates the distribution of lysine molecules within an individual peptide molecule, may be searched against a known proteome of a given organism to identify the individual peptide molecule. Such information may be used to identify a macromolecule (e.g., protein) from which the peptide was derived, and/or may preclude the need to identify all amino acids of the peptide.

Peptide sequencing may be used to acquire information (including, for example, amino acid sequence information) from individual peptide molecules in a mixture of diverse peptide molecules. In a non-limiting example, a plurality of peptides may be immobilized on a solid surface (including, for example, a glass slide, or a glass slide whose surface has been chemically modified, a plastic slide, a multi-well plate, a cassette), amino acids from the plurality of peptides may be coupled to fluorescent reporter moieties, and/or the fluorescent reporter moieties may be optically detected.

Numerous commercially available optical devices can be applied in or adapted for this manner. For example, conventional microscopes equipped with total internal reflection illumination and/or intensified charge-couple device (CCD) detectors may be adapted for sequencing methods disclosed herein. A high sensitivity CCD camera may be configured to simultaneously record the fluorescence intensity of multiple individual (e.g., single) peptide molecules distributed across a surface, and/or may be coupled to an image splitter to facilitate the simultaneous collection of multiple, distinct images (e.g., a first image comprising light of a first wavelength and/or a second image comprising light of a second wavelength). Using a motorized microscope stage with automated focus control to image multiple stage positions in the flow cell may allow thousands or more (e.g., millions) of individual single peptides (or more) to be sequenced in a single experiment.

The term “sequencing by degradation”, as used herein, refers to a method for analyzing a biomolecule comprising: (a) providing a polypeptide, wherein the polypeptide comprises at least one labeled internal amino acid; (b) detecting at least one signal or signal change from the polypeptide to identify at least a portion of a sequence of the polypeptide; and/or (c) subjecting the polypeptide to conditions sufficient to remove at least one amino acid from the polypeptide. In some embodiments, the polypeptide is immobilized to a support. In some embodiments, at least one amino acid is removed from an N-terminus of the polypeptide. In some embodiments, subsequent to (c), the at least one labeled internal amino acid becomes a labeled terminal amino acid. In some embodiments, the at least one labeled internal amino acid is from a plurality of labeled amino acids, wherein at least one signal or signal change comprises a collective signal from the plurality of labeled amino acids. In some embodiments, the plurality of labeled amino acids comprise amino acids with different labels. In some embodiments, the different labels generate signals with different signal patterns. In some embodiments, the at least one labeled internal amino acid comprises one or more members selected from the group consisting of lysine, glutamate, and aspartate. In some embodiments, the at least one labeled internal amino acid comprises an amino acid having a label covalently attached thereto, which label generates the at least one signal or signal change. In some embodiments, the at least one labeled internal amino acid comprises an amino acid having a dye coupled thereto, which dye generates the at least one signal or signal change. In some embodiments, the at least one signal or signal change is an optical signal.

In some embodiments, the at least one signal or signal change comprises a plurality of signals of different intensities. In some embodiments, the at least one signal or signal change comprises a plurality of signals of different frequencies or frequency ranges. In some embodiments, the at least one amino acid is removed from the polypeptide by a degradation reaction. In some embodiments, the degradation reaction is Edman degradation. In some embodiments, the at least one signal or signal change is detected with an optical detector having single-molecule sensitivity. In some embodiments, In some embodiments, the method further comprises processing at least a portion of the sequence against a reference sequence to identify the polypeptide or a protein from which the polypeptide is derived. In some embodiments, the method further comprises, subsequent to (c), (i) identifying the at least the portion of the sequence of the polypeptide to identify the polypeptide, and/or (ii) using the polypeptide identified in (i) to quantify the polypeptide or a protein from which the polypeptide was derived. In some embodiments, in (a), less than all amino acids of the polypeptide are labeled. In some embodiments, the method further comprises (i) repeating (b) and/or (c) to detect at least one additional signal or signal change from the polypeptide and/or (ii) using the at least one signal or signal change and/or the at least one additional signal or signal change to identify the at least the portion of the sequence.

As used herein, the term “fluorescence” refers to the emission of visible light by a substance that has absorbed light of a different wavelength. In some embodiments, fluorescence provides a non-destructive means of tracking and/or analyzing biological molecules based on the fluorescent emission at a specific wavelength. Proteins (including antibodies), peptides, nucleic acid, oligonucleotides (including single stranded and/or double stranded primers) may be “labeled” with a variety of extrinsic fluorescent molecules referred to as fluorophores. Isothiocyanate derivatives of fluorescein, such as carboxyfluorescein, are an example of fluorophores that may be conjugated to proteins (such as antibodies for immunohistochemistry) or nucleic acids. In some embodiments, fluorescein may be conjugated to nucleoside triphosphates and/or incorporated into nucleic acid probes (such as “fluorescent-conjugated primers”) for in situ hybridization. In some embodiments, a molecule that is conjugated to carboxyfluorescein is referred to as “FAM-labeled”.

In an aspect, the present disclosure provides solutions to the aforementioned challenges by providing expeditious and/or facile methods for analyzing a polypeptide. Additionally, some aspects of the present disclosure provide compositions that facilitate effective peptide characterization and/or analysis. Furthermore, in some aspects the present disclosure provides kits which enable effective polypeptide analysis.

### Biomolecule Barcodes

Molecular barcoding may be utilized for single molecule tracking, identification, or characterization in a wide range of applications. In a molecular barcoding method, a species may be coupled or provided coupled to a barcode. The barcode may comprise information in the form of a sequence, a composition, a recognizable chemical feature (e.g., an optically detectable label), a physical property (e.g., isoelectric point), or any combination therein. In some embodiments, the barcode comprises information in the form of an optically detectable label. In coupling the barcode to the species, the species may carry the information contained by the barcode. The information may be used to identify point of origin, such as the cell, volume, or emulsion from which a species was generated; a chemical treatment, such as a reaction or condition to which the species was subjected; an interaction, such as a transient binding event with a second species; or any combination thereof. Barcode information may be extracted by analyzing (e.g., sequencing or identifying an electrophoretic mobility of) the barcode. In this way, barcoding can increase the amount of information derived from an experiment or process.

In some embodiments, a peptide barcode can be used to identify a biomolecule. In some embodiments, a peptide barcode can be used to quantify a biomolecule. In some embodiments, a peptide barcode can be used to identify a property or characteristic of a biomolecule. In some embodiments, a peptide barcode can be used to identify a biomolecule's point of origin. In some embodiments, a peptide barcode can be used to identify a reaction or condition to which the biomolecule was subjected. In some embodiments, a peptide barcode can be used to identify a post-translational modification to which the biomolecule was subjected. In some embodiments, a peptide barcode can be used to quantify an amount of a biomolecule. In some embodiments, a peptide barcode can be used to identify a biomarker of a biomolecule. In some embodiments, a peptide barcode can be used to quantify a biomarker of a biomolecule.

However, barcoding methods can comprise a range of limitations. Many barcodes are limited to low information storage density. For example, with only four canonical nucleotide types, DNA barcodes typically encode a maximum of two bits per nucleotide, or about 6×10⁵ bits per millimeter, rendering complex information (e.g., the identities of multiple chemical steps enacted upon a single molecule) encoding unfeasible in many applications. Furthermore, many barcodes require mild chemical and/or physical conditions, limiting the types of solvents, acidities, salinities, temperatures, and/or reactant strengths a barcoded molecule may be subjected to. For example, DNA barcodes typically impose strict pH requirements to prevent phosphodiester hydrolysis, and thus barcode degradation. Similarly, many barcodes affect the physical and/or chemical properties of the species to which they are bound. In particular, DNA barcodes often impart strict solubility and/or isoelectric points upon species to which they couple.

The present disclosure overcomes these limitations by providing peptide barcodes capable of dense information storage, high stabilities, and/or minimal conveyance of chemical and/or physical properties upon the species to which they tether. Unlike nucleic acids, which typically comprise 4 canonical base types, peptides may readily incorporate a vast library of monomer units, including the 20 proteinogenic amino acids, hundreds of post-translationally modified amino acid types, and/or billions of synthetically derivable amino acid variants. Peptides may thus comprise a large amount of information per monomer (e.g., amino acid residue) unit. Furthermore, peptide backbones, which typically comprise repeating amide units, provide a considerable degree of stability, and/or thus may confer a tolerance to extreme physical and/or chemical conditions.

Disclosed herein is a method for identifying a biomolecule, the method comprising: (a) providing the biomolecule having coupled thereto an oligomeric barcode, wherein the oligomeric barcode comprises a plurality of monomeric subunits, wherein at least a subset of the monomeric subunits comprise a label; and/or (b) identifying the label, wherein the identifying is by sequencing by degradation. In some embodiments, the biomolecule is a polypeptide. In some embodiments, the biomolecule is a protein. In some embodiments, the method further comprises coupling the oligomeric barcode to the biomolecule.

A peptide barcode may be inert in a given method, system, or composition. For example, an assay which subjects a subject species (e.g., a molecule) to oxidizing and/or reducing conditions may utilize peptide barcodes devoid of cysteine residues, thereby avoiding disulfide formation via oxidizing reagent consumption and/or disulfide cleavage via reducing reagent consumption. An assay utilizing a strong electrophile (e.g., molecular chlorine) may utilize peptide barcodes devoid of nucleophilic amino acid residues (e.g., cysteine). Peptide barcodes may be designed (e.g., computationally or through a directed evolution process) to not comprise an affinity for species present in a method or assay. For example, an assay utilizing a plurality of enzymes may utilize a plurality of peptide barcodes with negligible antagonistic behaviors or affinities for the plurality of enzymes. A peptide barcode may be considered inert in a specific method, composition, or system when the barcode does not comprise, or comprises negligible, reactivity, agonistic, antagonistic, catalytic, binding, signaling, or inhibitory behavior.

A peptide may also be configured to minimally impact the chemical and/or physical properties of a barcoded molecule. In some embodiments, the peptide barcodes of the disclosure can be resistant against an oxidation reaction. In some embodiments, the peptide barcodes of the disclosure can be resistant against an reduction reaction. In some embodiments, the peptide barcodes of the disclosure can be resistant against an enzymatic modification. In some embodiments, the peptide barcodes of the disclosure can be resistant against a chemical modification. In some embodiments, the peptide barcodes of the disclosure can be resistant against a cleavage. Peptides are capable of adopting a range of isoelectric points, solubilities (e.g., high or low organic solvent solubilities), and/or reactivities. Accordingly, a set of peptide barcodes may be optimized for a specific method or application. For example, distinct sets of peptide barcodes may be designed (e.g., computationally designed through QM/MM optimization) for a 37° C. yeast expression assay in a mildly acid medium and a 97° C. Sulfolobaceae assay conducted at low pH.

In some embodiments, a peptide barcode may impart chemical, physical, and/or biological properties upon a biomolecule to which it is coupled. In some embodiments, a peptide barcode may impart a chemical property upon a biomolecule to which it is coupled. In some embodiments, a peptide barcode may increase a pH of a biomolecule to which it is coupled. In some embodiments, a peptide barcode may decrease a pH of a biomolecule to which it is coupled. In some embodiments, a peptide barcode may increase solubility of the biomolecule. In some embodiments, a peptide barcode may decrease solubility of the biomolecule. In some embodiments, a peptide barcode may increase an isoelectric point of the biomolecule. In some embodiments, a peptide barcode may decrease an isoelectric point of the biomolecule. In some embodiments, a peptide barcode may modulate the charge or charge distribution of the biomolecule. In some embodiments, a peptide barcode may impart a physical property upon a molecule to which it is coupled. In some embodiments, a peptide barcode may increase a size of a biomolecule by adding amino acid residues. In some embodiments, a peptide barcode may modulate the secondary structure of the biomolecule. In some embodiments, a peptide barcode may modulate the tertiary structure of the biomolecule. In some embodiments, a peptide barcode may increase access to a reactive site of the biomolecule. In some embodiments, a peptide barcode may decrease access to a reactive site of the biomolecule. In some embodiments, a peptide barcode may impart a biological property upon a molecule to which it is coupled. In some embodiments, a peptide barcode may increase a stability of a biomolecule to which it is coupled. In some embodiments, a peptide barcode may decrease a stability of a biomolecule to which it is coupled. In some embodiments, a peptide barcode may increase a reactivity of a biomolecule to which it is coupled. In some embodiments, a peptide barcode may decrease a reactivity of a biomolecule to which it is coupled. In some embodiments, a peptide barcode may increase a ligand affinity of a biomolecule to which it is coupled. In some embodiments, a peptide barcode may decrease a ligand affinity of a biomolecule to which it is coupled.

In some cases, a peptide barcode may comprise a function. In some embodiments, a peptide barcode may be used to purify the biomolecule. In some embodiments, a peptide barcode may be used to enrich for the biomolecule. In some embodiments, a peptide barcode may be used to chemically modify the biomolecule. In some embodiments, a peptide barcode may be used to enzymatically modify the biomolecule. In some embodiments, a peptide barcode may be used to detect the biomolecule. In some embodiments, a peptide barcode may be used to quantify an amount of the biomolecule. In some embodiments, a peptide barcode may comprise or be coupled to a purification tag (e.g., a His- or FLAG-tag), a localization signal (e.g., a nuclear localization sequence or cellular export sequence), a fluorescent tag, an affinity tag, or a chemically or enzymatically modifiable tag (e.g., a tag configured for redox cycling). In some embodiments, a peptide barcode may comprise or be coupled to a purification tag. In some embodiments, a peptide barcode may comprise or be coupled to a fluorescent tag. In some embodiments, a peptide barcode may comprise or be coupled to a chemically modifiable tag. In some embodiments, a peptide barcode may comprise or be coupled to an enzymatically modifiable tag.

A peptide barcode may comprise a plurality of amino acid residues. The type, order, properties, and/or chemical modifications of the amino acid residues may comprise information which may be extracted by analyzing (e.g., sequencing) the peptide barcode. A peptide barcode may contain one or more proteinogenic amino acid residues (e.g., selected from the group consisting of alanine, arginine, asparagine, aspartic acid, cystine, glutamine, glutamic acid, glycine, histidine, isoleucine, leucine, lysine, methionine, phenylalanine, proline, serine, threonine, tryptophan, tyrosine, and valine). A peptide barcode may contain a post-translationally modified amino acid, such as a methylated amino acid, a hydroxylated amino acid, a citrullinated amino acid, an acylated amino acid, an amidated amino acid, a prenylated amino acid, a lipoylated amino acid, a flavinated amino acid, a succinylated amino acid, a malonated amino acid, a glycosylated amino acid, a sialylated amino acid, a halogenated (e.g., fluorinated, chlorinated, brominated, or iodinated) amino acid, a carboxylated amino acid, a decarboxylated amino acid, a nitrosylated amino acid, a phosphorylated amino acid, a sulfurylated amino acid, a cyclized amino acid, a biotinylated amino acid, or any combination thereof. A peptide barcode may comprise a synthetic amino acid, such as an enantiomer of a proteinogenic amino acid (e.g., a D-amino acid) or a chemically (e.g., post-translationally) modified amino acid; a heteroatomic substitution variant of a proteinogenic amino acid such as a silicon-containing amino acid analogue; a post-translationally modified amino acid such as phosphoserine, azidolysine, dehydroalanine, pyroglutamic acid, hydroxyproline, thiazolyl or oxazolyl histidine, or thiourea arginine; an amino acid comprising a non-canonical side chain, such as a methylnaphthalene unit; or an amino acid comprising an alternate backbone structure, such as a β-amino acid.

In some embodiments, the oligomeric barcode comprises a polymer. In some embodiments, the oligomeric barcode comprises a polypeptide. In some embodiments, the oligomeric barcode comprises from about 2 to about 30 amino acids. In some embodiments, the oligomeric barcode comprises a non-natural amino acid. In some embodiments, the plurality of monomeric subunits is a plurality of amino acids.

A peptide barcode may comprise from about one type of amino acid to about five types of amino acids, from about five types of amino acids to about ten types of amino acids, from about ten types of amino acids to about fifteen types of amino acids, from about fifteen types of amino acids to about twenty types of amino acids, from about twenty types of amino acids to about twenty five types of amino acids, or from about twenty five types of amino acids to about thirty types of amino acids. A peptide barcode may comprise at least one type of amino acid, at least two types of amino acids, at least three types of amino acids, at least four types of amino acids, at least five types of amino acids, at least six types of amino acids, at least seven types of amino acids, at least eight types of amino acids, at least nine types of amino acids, at least ten types of amino acids, at least eleven types of amino acids, at least twelve types of amino acids, at least thirteen types of amino acids, at least fourteen types of amino acids, at least fifteen types of amino acids, at least sixteen types of amino acids, at least seventeen types of amino acids, at least eighteen types of amino acids, at least nineteen types of amino acids, at least twenty types of amino acids, at least twenty five types of amino acids, or at least thirty types of amino acids. A peptide barcode may comprise about one type of amino acid, about two types of amino acids, about three types of amino acids, about four types of amino acids, about five types of amino acids, about six types of amino acids, about seven types of amino acids, about eight types of amino acids, about nine types of amino acids, about ten types of amino acids, about eleven types of amino acids, about twelve types of amino acids, about thirteen types of amino acids, about fourteen types of amino acids, about fifteen types of amino acids, about sixteen types of amino acids, about seventeen types of amino acids, about eighteen types of amino acids, about nineteen types of amino acids, about twenty types of amino acids, about twenty five types of amino acids, or about thirty types of amino acids. A peptide barcode may comprise a limited subset of amino acids, such as at most thirty types of amino acids, at most twenty five types of amino acids, at most twenty types of amino acids, at most eighteen types of amino acids, at most seventeen types of amino acids, at most sixteen types of amino acids, at most fifteen types of amino acids, at most fourteen types of amino acids, at most thirteen types of amino acids, at most twelve types of amino acids, at most eleven types of amino acids, at most ten types of amino acids, at most nine types of amino acids, at most eight types of amino acids, at most seven types of amino acids, at most six types of amino acids, at most five types of amino acids, at most four types of amino acids, at most three types of amino acids, at most two types of amino acids, or at most one type of amino acid (e.g., peptide barcodes of a set comprising a single type of amino acid may be differentiated by length or by other chemical functionalization coupled thereto). A peptide barcode may comprise non-amino acid moieties. In some embodiments, a peptide barcode may comprise a backbone unit that does not comprise an amide bond. In some embodiments, a peptide barcode may comprise a backbone comprising a non-natural amino acid. In some embodiments, a peptide barcode may comprise a thiobutyric acid backbone unit.

### Peptide Barcode Libraries

Various aspects of the present disclosure provide peptide barcode libraries. A peptide barcode library may comprise a plurality of peptide barcodes. A peptide barcode library may also comprise a set of peptide barcodes which may be combinatorially generated from a particular set of amino acids or peptide fragments. Peptides of a peptide barcode library may comprise a common sequence. For example, all peptides of a peptide barcode library may comprise a common C-terminal sequence and/or a variable N-terminal sequence. The common sequence may comprise information regarding all members of peptide barcode library, such that a plurality of peptide barcode libraries may be distinguished by their common sequence regions.

A peptide barcode library may comprise a plurality of uniquely identifiable peptide barcodes. The number of uniquely identifiable peptide barcodes in the peptide barcode library may be equal to the number of peptide barcodes (i.e., all peptide barcodes in the peptide barcode library are uniquely identifiable). In some embodiments, only a portion of the peptide barcodes in the peptide barcode library may be uniquely identifiable peptide barcodes. In some cases, only a subset of subunits of a peptide barcode comprises identifiable information. For example, an assay may only identify chemically labeled side chains of peptide barcodes. Similarly, a peptide barcode library may generate a limited number of distinguishable fragments or tandem mass spectrometric fingerprints in a mass spectrometric assay.

A peptide barcode library may comprise a set of barcodes which provide a common signal or set of signals in a particular type of assay, which herein may collectively be referred to as a uniquely identifiable peptide barcode. A peptide barcode library may be taken to comprise as many uniquely identifiable peptide barcodes as are combinatorially achievable given the library peptide barcode structure or may be taken to comprise as many uniquely identifiable peptide barcodes as are physically present within a system. A peptide barcode library may comprise a relatively small number of uniquely identifiable peptide barcodes. Such a peptide barcode library may be used, for example, to classify a plurality of molecules into a finite number of categories, for example to identify the chromosome of origin for a plurality of gene products and/or may be generated with a relatively small subset of identifiable amino acids or amino acid sequences. For example, a peptide barcode library may comprise 6 uniquely identifiable amino acids or peptide sequences over 3 separate positions, and/or thereby comprise 216 uniquely identifiable peptide barcodes.

A peptide barcode library may comprise from about 5% to about 99.9% of uniquely identifiable peptide barcodes. In some embodiments, a peptide barcode library may comprise from about 5% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, from about 90% to about 95%, or from about 95% to about 99.9% of uniquely identifiable peptide barcodes. In some embodiments, a peptide barcode library may comprise at least about 5%, at least about 10%, at least about 20%, at least about 30%, at least about 40%, at least about 50%, at least about 60%, at least about 70%, at least about 80%, at least about 90%, at least about 95%, or at least about 99.9% of uniquely identifiable peptide barcodes. In some embodiments, a peptide barcode library may comprise about 5%, about 10%, about 20%, about 30%, about 40%, about 50%, about 60%, about 70%, about 80%, about 90%, about 95%, or about 99.9% of uniquely identifiable peptide barcodes. In some embodiments, a peptide barcode library may comprise at most about 5%, at most about 10%, at most about 20%, at most about 30%, at most about 40%, at most about 50%, at most about 60%, at most about 70%, at most about 80%, at most about 90%, at most about 95%, or at most about 99.9% of uniquely identifiable peptide barcodes.

A peptide barcode library may comprise from about 1 to about 5, from about 5 to about 10, from about 10 to about 15, from about 15 to about 20, from about 20 to about 50, from about 50 to about 100, from about 100 to about 200, from about 200 to about 300, from about 300 to about 400, from about 400 to about 500, from about 500 to about 750, from about 750 to about 1000, from about 1000 to about 1500, from about 1500 to about 2000, from about 2000 to about 2500, from about 2500 to about 3000, from about 3000 to about 4000, from about 4000 to about 5000, from about 5000 to about 6000, from about 6000 to about 8000, from about 8000 to about 10⁴, from about 10⁴ to about 5×10⁴, from about 5×10⁴ to about 10⁵, from about 10⁵ to about 5×10⁵, from about 5×10⁵ to about 10⁶, from about 10⁶ to about 5×10⁶, from about 5×10⁶ to about 10⁷, from about 10⁷ to about 5×10⁷, from about 5×10⁷ to about 10⁸, from about 10⁸ to about 5×10⁸, from about 5×10⁸ to about 10⁹, from about 10⁹ to about 5×10⁹, from about 5×10⁹ to about 10¹⁰, from about 10¹⁰ to about 5×10¹⁰, from about 5×10¹⁰ to about 10¹¹, from about 10¹¹ to about 5×10¹¹, from about 5×10¹¹ to about 10¹², from about 10¹² to about 5×10¹², or from about 5×10¹² to about 10¹³ uniquely identifiable peptide barcodes.

A peptide barcode library may comprise a single identifiable peptide barcode or at most about 2, at most about 3, at most about 4, at most about 5, at most about 6, at most about 8, at most about 10, at most about 12, at most about 15, at most about 20, at most about 30, at most about 40, at most about 50, at most about 100, at most about 200, at most about 400, at most about 600, at most about 1000, at most about 1500, at most about 2000, at most about 2500, at most about 3000, at most about 4000, at most about 5000, at most about 6000, at most about 8000, at most about 10⁴, at most about 5×10⁴, at most about 10⁵, at most about 5×10⁵, at most about 10⁶, at most about 5×10⁶, at most about 10⁷, at most about 5×10⁷, at most about 10⁸, at most about 5×10⁹, at most about 10⁹, at most about 5×10⁹, at most about 10¹⁰, at most about 5×10¹⁰, at most about 10¹¹, at most about 5×10¹¹, at most about 10¹², at most about 5×10¹², or at most about 10¹³ uniquely identifiable peptide barcodes. A peptide barcode library may comprise a single identifiable peptide barcode or about 2, about 3, about 4, about 5, about 6, about 8, about 10, about 12, about 15, about 20, about 30, about 40, about 50, about 100, about 200, about 400, about 600, about 1000, about 1500, about 2000, about 2500, about 3000, about 4000, about 5000, about 6000, about 8000, about 10⁴, about 5×10⁴, about 10⁵, about 5×10⁵, about 10⁶, about 5×10⁶, about 10⁷, about 5×10⁷, about 10⁸, about 5×10⁸, about 10⁹, about 5×10⁹, about 10¹⁰, about 5×10¹⁰, about 10¹¹, about 5×10¹¹, about 10¹², about 5×10¹², or about 10¹³ uniquely identifiable peptide barcodes. A peptide barcode library may comprise at least about 2, at least about 3, at least about 4, at least about 5, at least about 6, at least about 8, at least about 10, at least about 12, at least about 15, at least about 20, at least about 30, at least about 40, at least about 50, at least about 100, at least about 200, at least about 400, at least about 600, at least about 1000, at least about 1500, at least about 2000, at least about 2500, at least about 3000, at least about 4000, at least about 5000, at least about 6000, at least about 8000, at least about 10⁴, at least about 5×10⁴, at least about 10⁵, at least about 5×10⁵, at least about 10⁶, at least about 5×10⁶, at least about 10⁷, at least about 5×10⁷, at least about 10⁸, at least about 5×10⁸, at least about 10⁹, at least about 5×10⁹, at least about 10¹⁰, at least about 5×10¹⁰, at least about 10¹¹, at least about 5×10¹¹, at least about 10¹², at least about 5×10¹², at least about 10¹³, at least about 10¹⁴, at least about 10¹⁵, at least about 10¹⁶, at least about 10¹⁸, at least about 10²⁰, at least about 10²², at least about 10²⁴, at least about 10³⁰, at least about 10⁴⁰, or at least about 10⁵⁰ uniquely identifiable peptide barcodes. For example, a peptide barcode library may comprise 5 types of distinguishable amino acids (e.g., by fluorosequencing or by nanopore-based sequencing) in each of 100 separate positions, corresponding to a library size of nearly 10⁷⁰ uniquely identifiable peptide barcodes.

### Peptide Barcoding Methods

A peptide barcode may be coupled to a biomolecule. The biomolecule may be a small molecule such as a primary or secondary metabolite, a saccharide, a lipid, an amino acid, a nucleotide, a hormone, or any combination thereof. The biomolecule may be a macromolecule, such as a protein, a nucleic acid, a polysaccharide such as chitin, or any combination thereof. The peptide barcode may be coupled to a molecule in vivo, ex vivo, or in vitro.

A peptide barcode may be chemically coupled to a molecule. For example, a peptide barcode comprising a serine or threonine N-terminus may be oxidized (e.g., with a periodate oxidizing agent) to form an electrophilic glyoxylyl group configured for a wide range of molecular coupling steps. In some embodiments, the coupling comprises enzymatic ligation. In some embodiments, the coupling comprises transesterification. In some embodiments, the coupling comprises chemical coupling or enzymatic coupling. In some embodiments, the coupling comprises expressing the biomolecule coupled to the oligomeric barcode or co-translation of the oligomeric barcode as a peptide tag. In some embodiments, the coupling comprises expressing the biomolecule coupled to the oligomeric barcode. In some embodiments, the coupling comprises chemically synthesizing the biomolecule having coupled thereto the oligomeric barcode. A peptide barcode may be coupled to an N-terminus, a C-terminus, or an internal amino acid (e.g., directly or coupled to a linker or label) or a peptide. A peptide barcode may be coupled to a peptide with a peptide ligase (e.g., an omniligase). A molecule may be synthesized coupled to a peptide barcode (e.g., a starting material or intermediate comprises the peptide barcode). A molecule may be coupled to a single peptide barcode or to a plurality of peptide barcodes optionally comprising different information.

In some embodiments, the oligomeric barcode is coupled to the biomolecule via an N-terminal tag, a C-terminal tag, or an amino acid sidechain. In some embodiments, the N-terminal tag is a purification tag, a localization signal, a fluorescent tag, a chemically modifiable tag, or an enzymatically modifiable tag. In some embodiments, the C-terminal tag is a purification tag, a localization signal, a fluorescent tag, a chemically modifiable tag, or an enzymatically modifiable tag.

A barcode may be decoupled (e.g., cleaved) from a molecule. A barcode may be coupled to The biomolecule by a label or a linker, which may comprise a cleavable moiety (e.g., a thiocarbonate) that enables decoupling of the barcode from The biomolecule. The label or the linker may comprise a protease cleavage site, for example for TEV protease, trypsin, or any protease listed in TABLE 1. The label or the linker may be heat, acid, or photocleavable.

A peptide barcode may be cleaved from a molecule. The peptide barcode may be coupled to The biomolecule by a cleavable bond or linker. The cleavable bond or linker may comprise a chemically cleavable moiety, for example a reductively cleavable disulfide bridge; an enzymatically cleavable moiety, such as a glycoside hydrolase-cleavable saccharide linker or a reductase-cleavable polyphenol; a photocleavable moiety, such as a photocleavable benzylester or benzylcarbamate; a catalytically cleavable moiety, such as a copper-cleavable 1,2-diketone; or any combination thereof. In cases where a peptide barcode is directly coupled to a molecule, the peptide barcode may be coupled to The biomolecule by a cleavable bond or may itself comprise a cleavable moiety or protease cleavage site. A peptide barcode may comprise a protease cleavage site, enabling enzymatically mediated cleavage from a substrate. In some embodiments, the cleavable linker comprises a TEV protease cleavage site, a thrombin cleavage site, an enterokinase cleavage site, or any combination thereof. In some embodiments, the cleavable linker comprises an amino acid cleavage sequence not present in the oligomeric barcode. In some embodiments, the cleavable linker comprises a chemically cleavable group. In some embodiments, the chemically cleavable comprises a disulfide. In some embodiments, wherein the method further comprises cleaving the oligomeric barcode from the biomolecule.

A barcode may be separated from the biomolecule subsequent to decoupling or cleavage. A barcode may comprise a physical or chemical property that enables its separation from the species which it was cleaved, or from the sample from which it was derived. In some embodiments, a barcode can be separated based on a physical property. In some embodiments, a barcode can be separated based on a chemical property. In some embodiments, a barcode can be separated by differences in isoelectric points. In some embodiments, a barcode can be separated based on differences in charge or charge distribution. In some embodiments, a barcode can be separated based on differences in solubility. In some embodiments, a barcode can be separated based on differences in mass. In some embodiments, a barcode can be separated based on differences in shape or size. In some embodiments, a barcode can be separated based on differences in ligand affinity. In some embodiments, a barcode can be separated based on differences in surface affinity. In some embodiments, wherein the method further comprises separating the oligomeric barcode from the biomolecule after the cleaving. In some embodiments, wherein the separating comprises isoelectric focusing. In some embodiments, wherein the separating comprises chromatographic separation. In some embodiments, wherein the separating comprises electrophoretic separation.

A barcode may be collected by extraction (e.g., solvent extraction), isoelectric focusing, electrophoretic separation, chromatographic separation, affinity-based separation, flow-based separation, filter-based separation, precipitation-based separation, or any combination thereof. A barcode library may comprise a plurality of barcodes with a plurality of different chemical or physical properties, such that individual barcodes may be separated from the plurality of barcodes, or such that the plurality of barcodes may be separated into distinct groups. In some embodiments, a first subset of barcodes with a first property are separated from a second subset of barcodes with a second property. In some embodiments, a first subset of barcodes are separated from a second subset of barcodes based on differences in solubility. In some embodiments, a first subset of barcodes are separated from a second subset of barcodes based on differences in mass. In some embodiments, a first subset of barcodes are separated from a second subset of barcodes based on differences in isoelectric points. In some embodiments, a first subset of barcodes are separated from a second subset of barcodes based on differences in ligand affinity. In some embodiments, a first subset of barcodes are separated from a second subset of barcodes based on differences in surface affinity.

A barcode may comprise or be coupled to an enrichment tag, such as a peptide enrichment tag (e.g., a FLAG tag, a HIS tag, or a Myc tag), thereby providing a handle for affinity purification. The enrichment tag may be disposed between the barcode and a species to which it is coupled. For example, an antibody may be coupled to a peptide barcode by an S-tag.

A peptide barcode may be immobilized prior to analysis. The peptide barcode may be immobilized subsequent to cleavage from a species. The peptide barcode may be separated from a species or sample subsequent to cleavage by selective immobilization. The peptide barcode may be analyzed prior to, during, or subsequent to immobilization.

In some embodiments, wherein the method further comprises coupling the oligomeric barcode to a substrate after the cleaving. In some embodiments, wherein the method further comprises coupling the oligomeric barcode to a substrate after the separating. In some embodiments, wherein the oligomeric barcode is selected from a library comprising at least 216 uniquely identifiable oligomeric barcodes. In some embodiments, wherein the identifying comprises a resolution capable of resolving a single oligomeric barcode. In some embodiments, wherein the biomolecule and the oligomeric barcode comprise a common sequence.

The barcode may comprise information, such as the cell or sample of origin of The biomolecule, the structure or a portion of the structure of the biomolecule, a sequence of The biomolecule (e.g., an amino acid sequence of a peptide or a sequence of polyketide structural subunits), a conformation of a molecule, an activity (e.g., an enzymatic activity) of a molecule, a chemical modification (e.g., a post transcriptional or a post translational modification), or any combination thereof. Information may be identified from a barcode by sequencing, compositional analysis (e.g., elemental or amino acid composition), chromatography, electrophoresis, optical analysis, mass spectrometric analysis, or any combination thereof.

A barcode may be sequenced. In particular, the present disclosure provides a range of methods for rapid, high throughput peptide and/or peptide barcode sequencing, including mass spectrometry, nanopore translocation, Edman degradation (and similar terminal amino acid removal methods), N-terminal amino acid binding, nanogap impedance, nuclear magnetic resonance, fluorosequencing, and/or combinations thereof. In many cases, a peptide barcode sequence is identified with fluorosequencing, such that at least one type (e.g., lysine) or group (e.g., carboxylate side chain bearing) of amino acids are identified within the peptide barcode sequence. As fluorosequencing can distinguish a plurality of amino acid types, peptide barcode sequencing may generate one bit of information per identified amino acid or more than one bit of information per identified amino acid. For example, a peptide barcode may comprise a single type of labeled amino acid, such that fluorosequencing distinguishes labeled from unlabeled amino acids, and thereby generates one bit of information per identified amino acid (e.g., each amino acid of a sequence of the peptide barcode is identified as lysine or unlabeled). As a further example, a peptide barcode may comprise three types of labeled amino acids, and thereby generate 2 bits of information per identified amino acid (e.g., each position is identified as lysine, cysteine, tyrosine, or unlabeled).

A barcode may be expressed coupled to a biomolecule. For example, an organism may be stably transfected with a nucleic acid sequence encoding a peptide barcode (e.g., the nucleic acid sequence may be inserted into a host organism genome). An organism may be stably transfected with CRISPR-Cas mediated method, homologous recombination, or any combination thereof. A peptide barcode sequence may be inserted into a gene, such that the gene expresses a peptide coupled to the peptide barcode. The peptide barcode sequence may comprise a cleavable linker or an enrichment tag. A peptide barcode sequence may be inserted separately from a gene expressing a peptide to which it couples. For example, a recombinant organism may express a peptide barcode and/or an enzyme (e.g., a mutant glutathione transferase) capable of coupling the peptide barcode to a particular target. In some cases, a biomolecule is expressed coupled to a peptide barcode. For example, a protein may be expressed with a peptide barcode as a C-terminal tag. In some embodiments, a protein may be expressed with a peptide barcode as an N-terminal tag. In some embodiments, a protein may be expressed with a peptide barcode attached to an internal amino acid residue. For example, an organism may co-express a peptide barcode and/or a mutant enzyme (e.g., an engineered cytochrome P450) configured to couple the peptide barcode to a particular molecule or set of molecules, such as a steroid or a class of steroids. A molecule may be enzymatically ligated to a peptide barcode.

An organism may be transiently transfected with a nucleic acid sequence encoding a peptide barcode. A peptide barcode may be encoded by a vector. The vector may comprise a plasmid, a phagemid, a cosmid, a fosmid, or any combination thereof. The vector may also comprise a sequence encoding a species to which the peptide barcode may couple. For example, the vector may comprise a coding sequence comprising a protein and/or a peptide barcode, such that the protein is expressed coupled to the peptide barcode. The vector may comprise a sequence encoding an enrichment tag. The vector may comprise a sequence encoding a cleavable linker. The enrichment tag and/or cleavable linker may be expressed coupled to the peptide barcode. The sequence or sequences encoding the enrichment tag and/or the cleavable linker may be positioned between the sequence encoding the peptide barcode and the sequence encoding a peptide to which the peptide barcode is coupled. The vector may comprise a promoter. The vector may comprise a selection marker. Transfection with the vector may comprise DEAE-dextran-mediated transfected, electroporation, liposome-mediated transfection, calcium phosphate co-precipitation, calcium chloride co-precipitation, microinjection, or any combination thereof.

Disclosed herein is a plasmid encoding a polypeptide coupled to an oligopeptide barcode, the plasmid comprising an open reading frame downstream from a promoter, wherein the open reading frame comprises a sequence encoding the polypeptide and/or a sequence encoding the oligopeptide barcode, and/or wherein the oligopeptide barcode comprises a sequence that uniquely identifies the polypeptide. In some embodiments, the open reading frame further comprises a sequence encoding a cleavage site. In some embodiments, the sequence encoding the cleavage site is positioned between the sequence encoding the polypeptide and/or the sequence encoding the oligomeric peptide. In some embodiments, the sequence encoding cleavage site comprises a protease recognition sequence, and/or wherein the protease recognition sequence is not present in the sequence encoding the polypeptide.

In some embodiments, the protease recognition sequence comprises a TEV protease recognition sequence, a thrombin recognition sequence, an enterokinase recognition sequence, or any combination thereof. In some embodiments, the open reading frame further comprises a sequence encoding an enrichment tag. In some embodiments, the sequence encoding the enrichment tag is positioned between the sequence encoding the polynucleotide and/or the sequence encoding the oligomeric peptide. In some embodiments, the method further comprises a selection marker. In some embodiments, the method further comprises a promoter upstream of the open reading frame. In some embodiments, the promoter is a constitutive promoter.

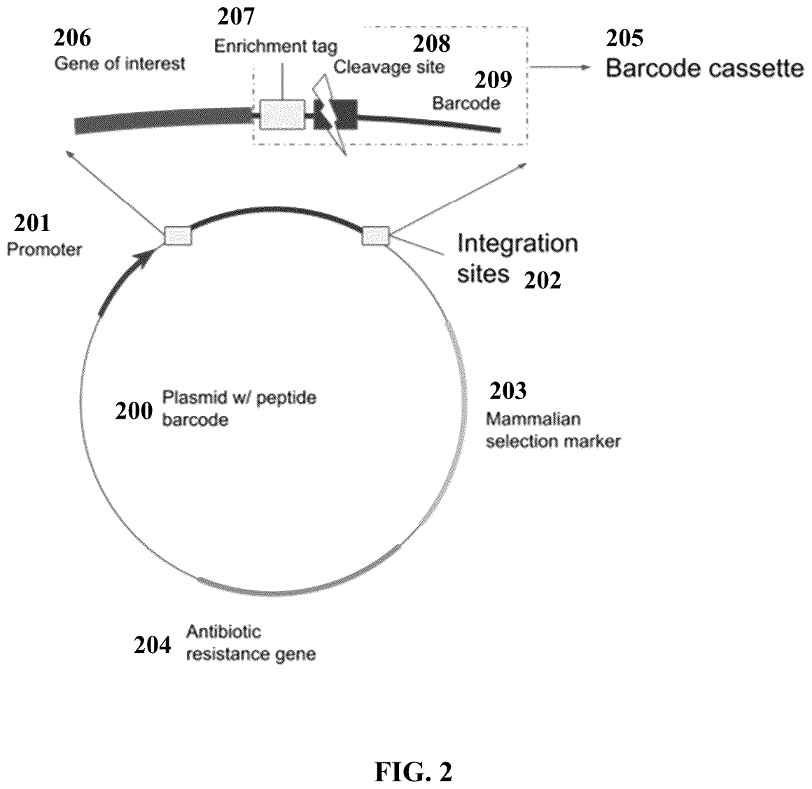

An example of a plasmid encoding a peptide barcode coupled to a peptide of interest is provided in FIG. 2. The plasmid with a peptide barcode (200) may comprise integration sites (202) configured to accept an open reading frame comprising a gene of interest (206) and/or a sequence encoding a peptide barcode (209), such that the gene product is expressed coupled to the peptide barcode. The peptide barcode sequence may be upstream of, downstream of, or inserted within the gene of interest (206), such that the peptide barcode may be expressed coupled to the C-terminus, N-terminus, or within the gene product. The open reading frame may further comprise a sequence encoding an enrichment tag (207) and/or a sequence encoding a cleavage site (208). In some cases, the sequence encoding the enrichment tag (207) and/or the sequence encoding the cleavage site (208) are disposed between the gene of interest (206) and the sequence encoding the peptide barcode (209). In other instances, the sequence encoding the enrichment tag (207) and/or the sequence encoding the cleavage site (208) may be disposed at an end of the open reading frame. As shown in FIG. 2, the enrichment tag (207), the sequence encoding the cleavage site (208), and/or the peptide barcode together form the barcode cassette (205). The open reading frame may be downstream of a promoter (201). The plasmid may further comprise a selection marker, such as an antibiotic resistance gene (204) and/or a mammalian selection marker (203). The plasmid may comprise an origin of replication.

### Peptide Barcode-Based Peptide Selection

In particular cases, a peptide barcode may comprise a sequence of a peptide or a protein to which the peptide or the protein is coupled, such that identification of a peptide-barcode sequence identifies a sequence of the peptide or the protein. For example, an antibody may comprise a peptide barcode comprising a sequence identical to at least a portion of one of complementarity determining regions (CDRs) of the antibody, such that identification of the peptide barcode sequence identifies the CDR of the antibody from which the barcode sequence is derived. The antibody or plurality of antibodies may comprise an IgA antibody, an IgD antibody, an IgE antibody, an IgG antibody, an IgM antibody, an IgW antibody, an IgY antibody, an IgNAR antibody, an hcIgG antibody, a camel Ig antibody, a minibody, a nanobody, a single domain antibody, a diabody, a triabody, or any combination thereof. A method may comprise an antibody selection method selected from the group consisting of antibody exclusion, affinity purification, antigen immobilization, selection on affinity capture antigens, physicochemical fractionation, and/or any combination thereof.

A peptide identification method may comprise expressing by an expression vector a peptide coupled to an oligomeric barcode. The vector may comprise a first sequence (e.g., a nucleotide sequence) encoding the peptide (e.g., a protein or an antibody) and/or a second sequence encoding a oligomeric barcode (e.g., an inert peptide barcode). A method may comprise transforming the vector to produce the peptide coupled to the oligomeric barcode, selecting the peptide (e.g., with an antigen display method), and/or identifying the peptide by identifying the oligomeric barcode coupled thereto. The method may further comprise cleaving the oligomeric barcode from the peptide. The method may comprise analyzing (e.g., sequencing) the oligomeric barcode. The method may comprise analyzing the vector (e.g., sequencing a plasmid vector). The method may further comprise immobilizing the oligomeric barcode (e.g., to a surface, such as a solid surface of a glass slide). The oligomeric barcode may be chemically or physically inert. The method may comprise a plurality of vectors each comprising a plurality of first sequences encoding a plurality of peptides and a plurality of second sequences encoding a plurality of oligomeric barcodes. The vector may comprise a plasmid, a phagemid, a cosmid, fosmid, or any combination thereof. The vector may comprise a sequence or a physical or chemical property enabling enrichment or isolation, such as an enrichment tag (e.g., a FLAG tag).

Disclosed herein is a method comprising: (a) providing a plurality of vectors, wherein each of the plurality of vectors comprises a first nucleotide sequence encoding a polypeptide and/or a second nucleotide sequence encoding a peptide barcode; (b) transforming the plurality of vectors to produce a plurality of polypeptides, wherein the polypeptide barcode is coupled to a polypeptide from the plurality of polypeptides; (c) selecting the polypeptide based on a condition; and/or (d) identifying the peptide barcode coupled to the polypeptide from the plurality of polypeptides, wherein the identifying is by sequencing by degradation.

In some embodiments, each of the plurality of vectors comprises a plasmid, a phagemid, a cosmid, fosmid, or any combination thereof. In some embodiments, each of the plurality of vectors further comprises a sequence encoding an enrichment tag. In some embodiments, each of the plurality of vectors further comprises a sequence encoding a cleavage tag, and/or wherein the cleavage tag is positioned between the first nucleotide sequence and/or the second nucleotide sequence. In some embodiments, each of the plurality of vectors comprises a promoter upstream of the first nucleotide sequence. In some embodiments, each of the plurality of vectors comprises a selection marker.

In some embodiments, the transforming comprises transient transfection, stable transfection, DEAE-dextran-mediated transfection, electroporation, liposome-mediated transfection, calcium phosphate co-precipitation, calcium chloride co-precipitation, microinjection, or any combination thereof. In some embodiments, the transforming comprises introducing the first nucleotide sequence and/or the second nucleotide sequence into a host organism genome. In some embodiments, the introducing comprises CRISPR-Cas enzymatic cleavage, homologous recombination, or any combination thereof.

In some embodiments, the peptide comprises an antibody. In some embodiments, the antibody comprises an IgA antibody, an IgD antibody, an IgE antibody, an IgG antibody, an IgM antibody, an IgW antibody, an IgY antibody, an IgNAR antibody, an hcIgG antibody, a camel Ig antibody, a minibody, a nanobody, a single domain antibody, a diabody, a triabody, or any combination thereof.

In some embodiments, the method further comprises cleaving the peptide barcode from the polypeptide. In some embodiments, the polypeptide barcode comprises a label. In some embodiments, the peptide barcode comprises a plurality of labels. In some embodiments, the plurality of labels comprises an amino acid specific label. In some embodiments, the plurality of labels comprises a methionine specific label, an arginine specific label, a histidine specific label, a tyrosine specific label, a carboxylic acid R-group specific label, a lysine specific label, a cysteine specific label, a tryptophan specific label, or any combination thereof. In some embodiments, the plurality of labels comprises a non-natural amino acid specific label. In some embodiments, the non-natural amino acid specific label is a phosphoserine specific label, phosphothreonine specific label, pyroglutamic acid specific label, hydroxyproline specific label, azidolysine specific label, or dehydroalanine specific label. In some embodiments, the label is a fluorescent label. In some embodiments, the label is a dye.

A peptide selection method may comprise fractionation across a biological barrier, such as a blood-brain barrier or an artificial analogue thereof. A peptide selection method may comprise function based screening or enzymatic inhibition screening. The vector may encode an unnatural amino acid in the barcoded sequence which could be manipulated to enrich or even detect the expressed proteins during the peptide selection step.

### Fluorosequencing

Fluorosequencing (e.g., sequencing by degradation) refers to sequencing peptides in a complex protein sample at the level of single molecules. In some embodiments, millions of individual fluorescently labeled peptides are visualized in parallel, monitoring changing patterns of fluorescence intensity as N-terminal amino acids are sequentially removed, and/or using the resulting fluorescence signatures (fluorosequences) to uniquely identify individual peptides. In some embodiments, amino acids are selectively labeled on immobilized peptides, and/or the amino acids are subjected to successive cycles of removing the peptide N-terminal residues (Edman degradation) and/or imaging the corresponding decrease of fluorescent intensity for individual peptide molecules. In some embodiments, amino acids are cleaved using chemical degradation, photochemical degradation, or enzymatic degradation. The methods of the present invention are capable of producing patterns sufficiently reflective of the peptide sequences to allow unique identification of a majority of proteins from a species. The resulting stair-step patterns of fluorescence decreases provide positional information of the select amino acid residues. This partial pattern is often sufficient to allow unique identification of the peptide by comparison to a reference proteome. The patterns of cleavage (even for a portion of the protein) provide sufficient information to identify a significant fraction of proteins within a known proteome, i.e. where the sequences of proteins are known in advance. In one embodiment, the single-molecule technologies of the present application allow the identification and/or absolute quantitation of a given peptide or protein in a biological sample.

In some embodiments, the methods disclosed herein can be used to perform large-scale sequencing (including but not limited to partial sequencing) of single intact peptides (not denatured) at the single molecule level by selective labeling amino acids on immobilized peptides followed by successive cycles of labeling and/or removal of the peptide amino-terminal amino acids. The methods and/or systems of the disclosure can identify amino acids in peptides, including peptides comprising unnatural amino acids. In one embodiment, the present invention comprises labeling the N-terminal amino acid with a first label and/or labeling an internal amino acid with a second label. In some embodiments, the labels are fluorescent labels. In other embodiments, the internal amino acid is Lysine. In other embodiments, amino acids in peptides are identified based on the fluorescent signature for each peptide at the single molecule level.

Various aspects of the present disclosure provide compositions and/or methods for peptide fluorosequencing, also called sequencing by degradation. A method consistent with the present disclosure may subject a peptide to fluorosequencing and/or an additional form of analysis. For example, a molecule of hemoglobin may be interrogated for glycation with immunostaining, and/or then subsequently digested and/or subjected to fluorosequencing for sequencing analysis. In one embodiment, the present invention provides a massively parallel and/or rapid method for identifying and/or quantitating individual peptide and/or protein molecules within a given complex sample.

In some embodiments, the methods of the disclosure comprises: (a) providing a polypeptide, wherein the polypeptide comprises at least one labeled internal amino acid; (b) detecting at least one signal or signal change from the polypeptide to identify at least a portion of a sequence of the polypeptide; and/or (c) subjecting the polypeptide to conditions sufficient to remove at least one amino acid from the polypeptide. In some embodiments, the at least one amino acid is removed from an N-terminus of the polypeptide. In some embodiments, subsequent to (c), the at least one labeled internal amino acid becomes a labeled terminal amino acid. In some embodiments, the at least one labeled internal amino acid is from a plurality of labeled amino acids, and/or wherein the at least one signal or signal change comprises a collective signal from the plurality of labeled amino acids. In some embodiments, the plurality of labeled amino acids comprise amino acids with different labels. In some embodiments, the different labels generate signals with different signal patterns.

In some embodiments, the at least one labeled internal amino acid comprises one or more members selected from the group consisting of lysine, glutamate, and aspartate. In some embodiments, the at least one labeled internal amino acid comprises an amino acid having a label covalently attached thereto, which label generates the at least one signal or signal change. In some embodiments, the at least one labeled internal amino acid comprises an amino acid having a dye coupled thereto, which dye generates the at least one signal or signal change. In some embodiments, the at least one signal or signal change is an optical signal. In some embodiments, the at least one signal or signal change is detected with an optical detector having single-molecule sensitivity. In some embodiments, the at least one signal or signal change comprises a plurality of signals of different intensities. In some embodiments, the at least one signal or signal change comprises a plurality of signals of different frequencies or frequency ranges.

In some embodiments, the label is coupled to an internal monomeric subunit of the plurality of monomeric subunits. In some embodiments, the label is an amino acid specific label. In some embodiments, the amino acid specific label comprises a methionine specific label, an arginine specific label, a histidine specific label, a tyrosine specific label, a carboxylic acid R-group specific label, a lysine specific label, a cysteine specific label, a tryptophan specific label, or any combination thereof. In some embodiments, the amino acid specific label comprises a non-natural amino acid specific label. In some embodiments, the non-natural amino acid specific label is a phosphoserine specific label, phosphothreonine specific label, pyroglutamic acid specific label, hydroxyproline specific label, azidolysine specific label, or dehydroalanine specific label. In some embodiments, the label is a fluorescent label. In some embodiments, the label is a dye.

In some embodiments, the at least one amino acid is removed from the polypeptide by a degradation reaction. In some embodiments, the degradation reaction is Edman degradation. In some embodiments, the method further comprises processing at least the portion of the sequence against a reference sequence to identify the polypeptide or a protein from which the polypeptide is derived. In some embodiments, the method further comprises, subsequent to (c), (i) identifying the at least the portion of the sequence of the polypeptide to identify the polypeptide, and/or (ii) using the polypeptide identified in (i) to quantify the polypeptide or a protein from which the polypeptide was derived. In some embodiments, in (a), less than all amino acids of the polypeptide are labeled. In some embodiments, the method further comprises (i) repeating (b) and/or (c) to detect at least one additional signal or signal change from the polypeptide and/or (ii) using the at least one signal or signal change and/or the at least one additional signal or signal change to identify the at least the portion of the sequence.

A characteristic feature of many fluorosequencing methods is coupling amino acid labels to a peptide to be sequenced. A label may be an amino acid specific label (e.g., configured to couple to a specific type of amino acid or a specific set of types of amino acids). A fluorosequencing method may comprise labeling a plurality of types of amino acids with separate, amino acid type specific labels. A fluorosequencing method may comprise labeling one, two, three, four, five, six, or more different types of amino acids residues in a subject peptide or protein. A plurality of amino acid residues may include, for example, an N-terminal amino acid, cysteine, lysine, glutamic acid, aspartic acid, tryptophan, tyrosine, serine, threonine, arginine, histidine, methionine, or any combination thereof. Each of these amino acid residues may be labeled with a different labeling moiety. Multiple amino acid residues may be labeled with the same labeling moiety such as (i) aspartic acid and/or glutamic acid or (ii) serine and/or threonine.

In one embodiment, a method of labeling a peptide comprises: a) providing, i) a peptide having at least one Cysteine amino acid, at least one Lysine amino acid, an N-terminal end, an amino acid having at least one carboxylate side group, a C-terminal end, and/or at least one Tryptophan amino acid, and/or ii) a first compound, iii) a second compound, iv) a third compound, v) a fourth compound, and/or vi) a fifth compound; and/or b) labeling the Cysteine with the first compound, c) labeling the Lysine with the second compound, d) labeling the N-terminal end with the third compound, e) labeling the carboxylate side group and/or the C-terminal end with the fourth compound; and/or f) labeling the Tryptophan with the fifth compound for providing a peptide having specific labels. In one embodiment, steps b-f are sequential in order from b-f. In one embodiment, the labeling in steps b-f is performed in one (a single) solution. In one embodiment, steps b-f are sequential in order from b-f and/or performed in one solution. In one embodiment, the first compound is iodoacetamide. In one embodiment, the second compound is 2-methylthio-2-imadazoline hydroiodide (MDI). In one embodiment, the third compound is 1-(4,4-dimethyl-2,6-dioxocyclohexylidene)-3-methylbutyl diethyl phosphate (Phos-ivDde). In one embodiment, the fourth compound is selected from the group consisting of benzylamine (BA), 3-dimethylaminopropylamine, and isobutylamine. In one embodiment, the fifth compound is 2,4-dinitrobenzenesulfenyl chloride.

In one embodiment, disclosed herein is a method of treating a peptide comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and/or the N-terminal amino acid of each peptide labeled with a second label, the second label being different from the first label; b) treating the plurality of immobilized peptides under conditions such that each N-terminal amino acid of each peptide is removed; and/or c) detecting the first signal for each peptide at the single molecule level. In one embodiment, the second label is attached via an amine-reactive dye. In one embodiment, the second label is selected from the group consisting of fluorescein isothiocyanate, rhodamine isothiocyanate or other synthesized fluorescent isothiocyanate derivative. In one embodiment, portions of the emission spectrum of the first label do not overlap with the emission spectrum of the second label. In one embodiment, the removal of the N-terminal amino acid in step b) is done under conditions such that the remaining peptides each have a new N-terminal amino acid. In one embodiment, the method further comprises the step d) adding the second label to the new N-terminal amino acids of the remaining peptides. In one embodiment, among the remaining peptides the new end terminal amino acid is Lysine. In one embodiment, the method further comprises the step e) detecting the next signal for each peptide at the single molecule level.

In one embodiment, the method further comprises a step of treating the immobilized peptides under conditions such that each N-terminal amino acid of each peptide is removed by an Edman degradation reaction; and/or a step of detecting the signal for each peptide at the single molecule level. In one embodiment, the label is attached to a fluorophore by a covalent bond. In one embodiment, the fluorophore and/or the covalent bond is resistant to degradation effects when incubated in an Edman degradation reaction solvent. In some embodiments, the fluorophore is a fluorophore that remains intact and/or attached to the label during Edman degradation sequencing.

The repetitive detection of signal for each peptide at the single molecule level results in a pattern. The resulting pattern is unique to a single-peptide within the plurality of immobilized peptides. In one embodiment, the single-peptide pattern is compared to the proteome of an organism to identify the peptide, one embodiment, the intensity of the labels are measured amongst the plurality of immobilized peptides. In some embodiments, the peptides are immobilized via Cysteine residues. In some embodiments, the detecting in step c) is done with optics capable of single-molecule resolution. In a specific embodiment, one or more of the plurality of peptides comprises one or more unnatural amino acids.

In some embodiments, the emission spectrum of the first label do not overlap with the emission spectrum of the second label. In some embodiments, the removal of the N-terminal amino acid in step b) is done under conditions such that the remaining peptides each have a new N-terminal amino acid. In one embodiment, the method further comprises the step d) adding the second label to the new N-terminal amino acids of the remaining peptides. In some embodiments, among the remaining peptides, the new end terminal amino acid is Lysine. In one embodiment, the method further comprises the step e) detecting the next signal for each peptide at the single molecule level. In one embodiment, the intensity of the first and/or second labels are measured amongst the plurality of immobilized peptides. In some embodiments, the peptides are immobilized via Cysteine residues. In some embodiments, the detecting in step c) is done with optics capable of single-molecule resolution. In one embodiment, one or more of the plurality of peptides comprises one or more unnatural amino acids. In one embodiment, the unnatural amino acids comprises moieties selected from the group consisting of hydroxycarboxylates, aldehydes, thiols, and olefins. In one embodiment, one or more of the plurality of peptides comprises one or more beta amino acids.

In one embodiment, the method further comprises a step of treating an immobilized peptide (e.g., a support or bead) under conditions such that each N-terminal amino acid of each peptide is removed by an Edman degradation reaction; and/or a step of detecting the signal for each peptide at the single molecule level. In some embodiments, the N-terminal amino acid removing step and/or the detecting step are successively repeated from about 1 time to about 5 times, from about 5 times to about 10 times, from about 10 times to about 20 times, from about 20 times to about 30 times, from about 30 times to about 40 times, from about 40 times to about 50 times, from about 50 times to about 60 times, from about 60 times to about 70 times, from about 70 times to about 80 times, from about 80 times to about 90 times, or from about 90 times to about 100 times. In some embodiments, the N-terminal amino acid removing step and/or the detecting step are successively repeated at least about 5 times, at least about 10 times, at least about 20 times, at least about 30 times, at least about 40 times, at least about 50 times, at least about 60 times, at least about 70 times, at least about 80 times, at least about 90 times, or at least about 100 times. In some embodiments, the N-terminal amino acid removing step and/or the detecting step are successively repeated about 5 times, about 10 times, about 20 times, about 30 times, about 40 times, about 50 times, about 60 times, about 70 times, about 80 times, about 90 times, or about 100 times. In some embodiments, the N-terminal amino acid removing step and/or the detecting step are successively repeated at most about 5 times, at most about 10 times, at most about 20 times, at most about 30 times, at most about 40 times, at most about 50 times, at most about 60 times, at most about 70 times, at most about 80 times, at most about 90 times, or at most about 100 times.

A label may comprise a detectable moiety. The detectable moiety (i.e., label) may be optically detectable (e.g., fluorescent, phosphorescent, luminescent, or light absorbing). The detectable moiety may be electrochemically detectable (e.g., a redox active moiety with a characteristic oxidation or reduction potential). The detectable moiety may comprise a mass tag (e.g., for identification with mass spectrometry. A detectable moiety may identify a label to which it is attached. A plurality of labels may comprise a plurality of detectable moieties which identify labels of the plurality of labels by their type. For example, a method may comprise a plurality of types of labels configured to couple to different amino acids, each comprising a different detectable moiety that uniquely identifies the label by its type.

Labeling specificity can be a major challenge for a fluorosequencing method. In many cases, a label may comprise reactivity toward a plurality of amino acid types. For example, some maleimide labels can react with cysteine, lysine, and/or N-terminal amines. Discriminating between similarly reactive amino acid residues can require precise ordering of labeling steps. In the above maleimide example, lysine may be discriminated from cysteine by first reacting cysteine with a cysteine specific labeling step (e.g., iodoacetamide coupling at pH 7-8), thereby preventing further cysteine labeling in a subsequent lysine labeling step. A method may comprise cysteine labeling prior to lysine labeling. A method may comprise cysteine labeling prior to aspartate and/or glutamate labeling. A method may comprise cysteine labeling prior to tryptophan labeling. A method may comprise cysteine labeling prior to tyrosine labeling. A method may comprise cysteine labeling prior to serine and/or threonine labeling. A method may comprise cysteine labeling prior to histidine labeling. A method may comprise cysteine labeling prior to arginine labeling. A method may comprise lysine labeling prior to glutamate labeling. A method may comprise lysine labeling prior to aspartate labeling. A method may comprise lysine labeling prior to tryptophan labeling. A method may comprise lysine labeling prior to tyrosine labeling. A method may comprise tyrosine labeling prior to lysine labeling. A method may comprise lysine labeling prior to serine and/or threonine labeling. A method may comprise lysine labeling prior to arginine labeling. A method may comprise carboxylate side chain (e.g., glutamate and/or aspartate side chain) labeling prior to tryptophan labeling. A method may comprise carboxylate side chain (e.g., glutamate and/or aspartate side chain) labeling prior to tyrosine labeling. A method may comprise carboxylate side chain (e.g., glutamate and/or aspartate side chain) labeling prior to serine labeling. A method may comprise carboxylate side chain (e.g., glutamate and/or aspartate side chain) labeling prior to serine and/or threonine labeling. A method may comprise carboxylate side chain (e.g., glutamate and/or aspartate side chain) labeling prior to histidine labeling. A method may comprise carboxylate side chain (e.g., glutamate and/or aspartate side chain) labeling prior to arginine labeling. A method may comprise C-terminal carboxylate labeling prior to lysine labeling. A method may comprise C-terminal carboxylate labeling prior to tyrosine labeling. A method may comprise C-terminal carboxylate labeling prior to histidine labeling. A method may comprise C-terminal carboxylate labeling prior to tryptophan labeling. A method may comprise C-terminal carboxylate labeling prior to glutamate and/or aspartate labeling. A method may comprise C-terminal carboxylate labeling prior to serine and/or threonine labeling. A method may comprise at least 2, at least 3, at least 4, at least 5, or at least 6 amino acid labeling steps performed in a sequence configured to minimize or prevent label cross-reactivity (e.g., labeling more than the intended type or types of amino acids). A method may comprise 2, 3, 4, 5, or 6 amino acid labeling steps performed in a sequence configured to minimize or prevent label cross-reactivity (e.g., labeling more than the intended type or types of amino acids).

Fluorosequencing may comprise removing peptides through techniques such as Edman degradation following or preceding subject peptide detection. Sequential peptide removal may generate sequence or position-specific information. For example, a reduction in fluorescence following an N-terminal amino acid removal step may indicate that a labeled amino acid, and/or thus that a specific type of amino acid, was disposed at a peptide N-terminal. Removal of each amino acid residue can be carried out with a variety of different techniques including Edman degradation and/or proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide chain. In situations where Edman degradation is used, the amino acid residue at the N-terminus of the peptide chain is removed.

In some embodiments, the sequencing by degradation comprises Edman degradation. In some embodiments, the sequencing by degradation comprises subjecting the oligomeric barcode to conditions sufficient to remove at least one monomeric subunit from the oligomeric barcode. In some embodiments, the sequencing by degradation comprises subjecting the oligomeric barcode to conditions sufficient to remove at least one amino acid from the oligomeric barcode. In some embodiments, the label generates at least one signal or at least one signal change. In some embodiments, the at least one signal or the at least one signal change is an optical signal. In some embodiments, the at least one signal or the at least one signal change comprises a plurality of signals of different intensities. In some embodiments, the at least one signal or the at least one signal change comprises a plurality of signals of different frequencies or signals of different frequency ranges.

In one embodiment, the label is attached to a fluorophore by a covalent bond. In one embodiment, the fluorophore and/or the covalent bond is resistant to degradation effects when incubated in an Edman degradation reaction solvent. A labeling moiety used in the instant application may be configured to withstand conditions for removing one or more of the amino acid residues. Some non-limiting examples of potential labeling moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and/or (5)6-napthofluorescein. In some embodiments, a labeling moiety is tetramethylrhodamine, Si-Rhodamine, Rhodamine B, Rhodamine B N, N′-dimethylethylenediamine, Rhodamine B sulfenyl chloride, Alexafluor555, Alexa Fluor 405, Atto647N, (5)6-napthofluorescein, variants and/or derivations thereof, etc. In one embodiment, the fluorophore is selected from the group consisting of tetramethylrhodamine, Si-Rhodamine, Rhodamine B, Rhodamine B N, N′-dimethyl ethylenediamine, Rhodamine B sulfenyl chloride, Alexafluor555, Alexa Fluor 405, Atto647N, (5)6-napthofluorescein, variants and/or derivations thereof. The labeling moiety may be a fluorescent peptide or protein or a quantum dot. In some embodiments, two-color single molecule peptide sequencing reactions can be used to identify and/or quantify biomolecules by using two or more fluorescent molecules.

In some embodiments, amino acids can be removed from the carboxy terminus of a biomolecule, revealing C-terminal sequences instead of N-terminal sequences. In some embodiments, an engineered carboxypeptidase is used to mimic Edman degradation. In some embodiments, the sequencing by degradation comprises enzymatic cleavage of the oligomeric barcode from the biomolecule. In some embodiments, the sequencing by degradation comprises chemical cleavage of the oligomeric barcode from the biomolecule. In some embodiments, the chemical cleavage comprises cyanogen bromide cleavage, BNPS-skatole cleavage, formic acid cleavage, hydroxylamine cleavage, 2-nitro-5-thiocyanobenzoic acid cleavage, or any combination thereof.

In some embodiments, the methods disclosed herein comprise identifying amino acids in peptides, comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and/or the N-terminal amino acid of each peptide labeled with a second label, the second label being different from the first label and/or selected from the group consisting of Alexa fluor dyes and Atto dyes, wherein a subset of the plurality of peptides comprise an N-terminal Lysine having both the first and/or second label; b) treating the plurality of immobilized peptides under conditions such that each N-terminal amino acid of each peptide is removed by an Edman degradation reaction; and/or c) detecting the first signal for each peptide at the single molecule level under conditions such that the subset of peptides comprising an N-terminal Lysine is identified. It is preferred that the removal of the N-terminal amino acid in step b) is done under conditions such that the remaining peptides each have a new N-terminal amino acid. The present invention further contemplates in one embodiment, a method of identifying amino acids in peptides, comprising: a) providing a plurality of peptides immobilized on a solid support, each peptide comprising an N-terminal amino acid and internal amino acids, the internal amino acids comprising Lysine, each Lysine labeled with a first label, the first label producing a first signal for each peptide, and/or the N-terminal amino acid of each peptide labeled with a second label, the second label being different from the first label and/or selected from the group consisting of Alexa fluor dyes and Atto dyes, wherein a subset of the plurality of peptides comprise an N-terminal acid that is not Lysine; b) treating the plurality of immobilized peptides under conditions such that each N-terminal amino acid of each peptide is removed by an Edman degradation reaction; and/or c) detecting the first signal for each peptide at the single molecule level under conditions such that the subset of peptides comprising an N-terminal amino acid that is not Lysine is identified. It is preferred that the removal of the N-terminal amino acid in step b) is done under conditions such that the remaining peptides each have a new N-terminal amino acid. It is preferred that the peptides are immobilized via Cysteine residues. In one embodiment, one or more of the plurality of peptides comprises one or more unnatural amino acids. In one embodiment, the unnatural amino acids comprise moieties selected from the group consisting of hydroxycarboxylates, aldehydes, thiols, and/or olefins, one embodiment, one or more of the plurality of peptides comprises one or more beta amino acids.

Detecting the immobilized peptide may comprise capturing an image comprising the peptide. The image may comprise a spatial address specific to the peptide. A plurality of peptides may be detected in a single image, wherein one or more of the peptides may comprise a spatial address within the image. The surface may be optically transparent across the visible spectrum and/or the infrared spectrum. The surface may possess a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 m thick, between 1 and 5 m thick, between 2 and 10 m thick, between 5 and 20 m thick, between 20 and 50 m thick, between 50 and 200 m thick, between 200 and 500 m thick, or greater than 500 m in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and/or metal surfaces (Gold coating)), coating schemes (spin-coating, dip-coating, electron beam deposition for metals, thermal vapor deposition and/or plasma enhanced chemical vapor deposition) and/or functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and/or modified targets for selection. Alternatively, an aminosilane modified surfaces may be used in the methods described herein. The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein may involve imaging the peptide or protein to determine the presence of one or more labeling moieties (e.g., amino acid labels) coupled to the peptide. The sequencing technique may comprise imaging a plurality of peptides or proteins to determine the presence of one or more labeling moieties on individual peptides from among the plurality of peptides. The sequencing technique may comprise imaging from about 10³ to about 10⁴, from about 10⁴ to about 10⁵, from about 10⁵ to about 10⁶, from about 10⁶ to about 10⁷, or from about 10⁷ to about 10⁸ proteins or peptides. The sequencing technique may comprise imaging at least about 10³, at least about 10⁴, at least about 10⁵, at least about 10⁶, at least about 10⁷, or at least about 10⁸ or more proteins or peptides (e.g., imaging a portion of a surface comprising at least about 10³ to at least about 10⁸ proteins or peptides). The sequencing technique may comprise imaging about 10³, about 10⁴, about 10⁵, about 10⁶, about 10⁷, or about 10⁸ or more proteins or peptides (e.g., imaging a portion of a surface comprising about 10³ to about 10⁸ proteins or peptides). The sequencing technique may comprise imaging at most about 10³, at most about 10⁴, at most about 10⁵, at most about 10⁶, at most about 10⁷, or at most about 10⁸ or more proteins or peptides (e.g., imaging a portion of a surface comprising at most about 10³ to at most about 10⁸ proteins or peptides).

These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (SEQ ID NO: 2) (wherein ‘K’ denotes lysine, ‘D’ denotes aspartate, ‘Y’ denotes tyrosine, ‘A’ denotes alanine, and ‘G’ denotes glycine), and/or may comprise labels coupled to each lysine and/or tyrosine residue. A first image comprising the C-terminal immobilized peptide may indicate the presence of two lysines and/or one tyrosine in the peptide. The N-terminal amino acid may be removed (e.g., by Edman degradation), such that a second image comprising the C-terminal immobilized peptide may indicate the presence of one lysine and/or one tyrosine in the peptide. This process may be repeated until a sequence of KXXYXXXXXXXKX (SEQ ID NO: 3) is identified for the peptide, wherein ‘X’ indicates a non-lysine, non-tyrosine amino acid, ‘K’ indicates a lysine, and ‘Y’ indicates a tyrosine. A method of the present disclosure can identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and/or comparing these locations to known peptide sequences, which may identify the entire list of amino acid residues in the peptide sequence. For example, identifying the positions of the lysines and/or cysteines in a 40 amino acid fragment of a human protein may uniquely identify the protein (e.g., only one human protein contains the specific pattern of lysine and/or cysteine residues identified in the 40 amino acid fragment).

An imaging method may involve a variety of different spectrophotometric and/or microscopy methods, such as fluorimetry, diffuse reflectance, interferometric scattering, Raman, resonance enhanced Raman, infrared absorbance, visible light absorbance, ultraviolet absorbance, and/or fluorescence. In some embodiments, a conventional microscope equipped with total internal reflection illumination and/or an intensified charge-couple device (CCD) detector may be used for imaging. Depending on the absorption and/or emission spectra of fluorescent Edman labels employed, appropriate filters can be used to record the emission intensity of the labels. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide. Such imaging methods may be used to determine the presence or absence of a label on a specific peptide sequence. After repeated cycles of removing an amino acid residue and/or imaging a subject peptide, the position of the labeled amino acid residue can be determined in the peptide.

For each Edman cycle, the fluorescence intensity of a label is recorded after each cleavage step. The loss and/or uptake of a label after each cleavage step and/or coupling step serves as a 1) counter for the number of amino acid residues removed, and/or 2) an internal error control indicating the successful completion of each round of Edman degradation for each immobilized peptide.

Following image processing to filter noise and/or identify the location of peptides, and/or to map the locations of the same peptides across the set of collected images, intensity profiles for labels are associated with each peptide as a function of Edman cycle. The label intensity profile of each error free peptide sequencing reaction is transformed into a binary sequence in which a “1” precedes a drop in fluorescence intensity and/or its location (i.e., position within the binary sequence). Identifies the number of Edman cycles performed. A database of predicted potential proteins is used as a reference database. The binary intensity profile of each peptide, as generated from the single molecule microscopy, is then compared to the entries in the simulated peptide database. Quantification can be accomplished by counting peptides derived from each protein observed.

### Selective Amino Acid Labeling

Various aspects of the present disclosure provide methods for selectively labeling amino acid types (e.g., lysine, tyrosine, or phosphotyrosine) or amino acid groups (e.g., carboxylate side chain-containing or aromatic side chain-containing). A composition or method of the present disclosure may selectively label cysteine, lysine, tyrosine, histidine, glutamic acid, aspartic acid, tyrosine, threonine, serine, arginine, N-terminal amines, C-terminal carboxyl groups, or any combination thereof. A composition or method may selectively label a group of amino acids, for example, a specific maleimide reagent may couple to lysine and/or cysteine residues present in a sample.

The free thiol group of a cysteine side chain is often the most nucleophilic group in a peptide (Scheme 1), and/or thus may promiscuously react with a range of reagents. To prevent such cross-reactivity, thiol side chains are often reacted early within labeling schemes in order to prevent or reduce the likelihood of further reactivity. An example of a thiol-selective reaction is an iodoacetamide coupling step. Such a reaction may be performed in pH ranges which limit (e.g., prevent) lysine cross-reactivity, such as a sufficiently low pH to ensure lysine protonation.

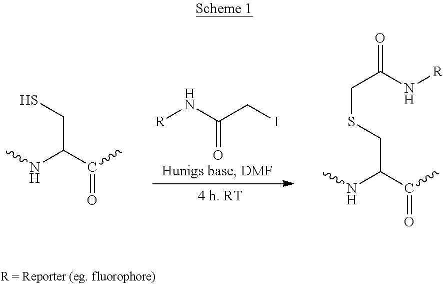

Scheme 2 provides an example of a lysine labeling reaction. The lysyl amine (e.g., a lysyl butylamine sidechain) can be selectively labeled with an ester (e.g., an NHS ester). Such a reaction may be performed after cysteine labeling in cases where cross-reactivity may be possible.

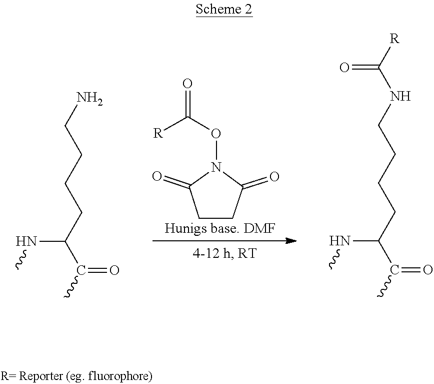

Peptide carboxylates may be labeled through amine coupling, an example of which is provided in Scheme 3. Carboxyl side chains (e.g., those of aspartic acid and/or glutamic acid) and/or C-terminal carboxyl groups can be converted to amides via amine-based nucleophilic substitution. The resulting amides may comprise detectable moieties, chemically inert groups, or reactive handles for further coupling. For example, an amine reagent for carboxylate amidation may comprise an alkyne suitable for a subsequent coupling step. In particular instances, a peptide is digested using Glu-C protease under pH 8 digestion buffer or a sufficiently similar protease/buffer system such that the cleavage site occurs on the C-terminal-side of an acidic residue (e.g., aspartic acid and/or glutamic acid). Such a digestion method can generate peptides in which every carboxyl residue (e.g., glutamic acid and/or aspartic acid) is disposed at a peptide C-terminus, thus enabling C-terminal selective amino acid immobilization. Alternate reactive groups can be used in place of an alkyne.

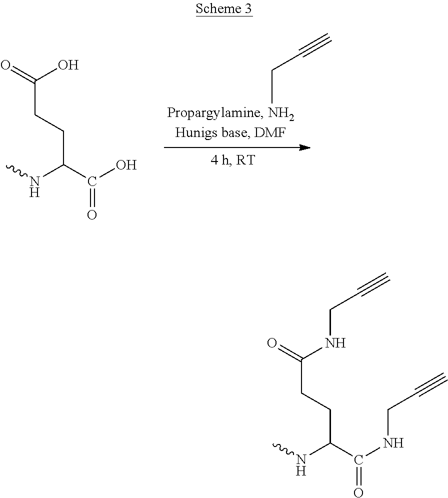

Scheme 4 provides an example of tyrosine-specific labeling scheme. The position adjacent (e.g., ortho to) the tyrosine phenol hydroxyl carbon can be labeled through a two-step labeling process using a bifunctional reagent. Following diazo-coupling to tyrosine, a second reagent (such as a dithiolane) may optionally be coupled to the diazo label (e.g., to selectively couple a detectable moiety to the labeled tyrosine). Alternatively, the diazonium reagent may comprise a detectable moiety or may lack chemically reactive handles for further coupling.

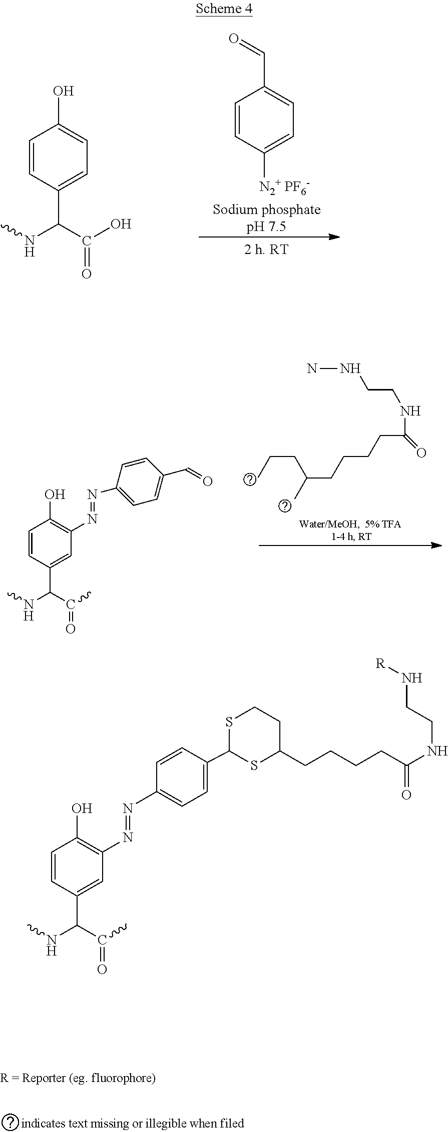

Scheme 5 provides an example of a histidine coupling scheme. A histidine imidazole nitrogen can be labeled through a two-step labeling process using an alpha-beta unsaturated carbonyl compound, such as 2-cyclohexenone. The alpha-beta unsaturated carbonyl compound may react with histidine in a nucleophilic addition reaction. The alpha-beta unsaturated carbonyl may comprise a detectable moiety. Following histidine coupling, the alpha-beta unsaturated carbonyl may be further coupled to an additional label, such as a dithiolane. Histidine may alternatively be selectively coupled to an epoxide reagent.

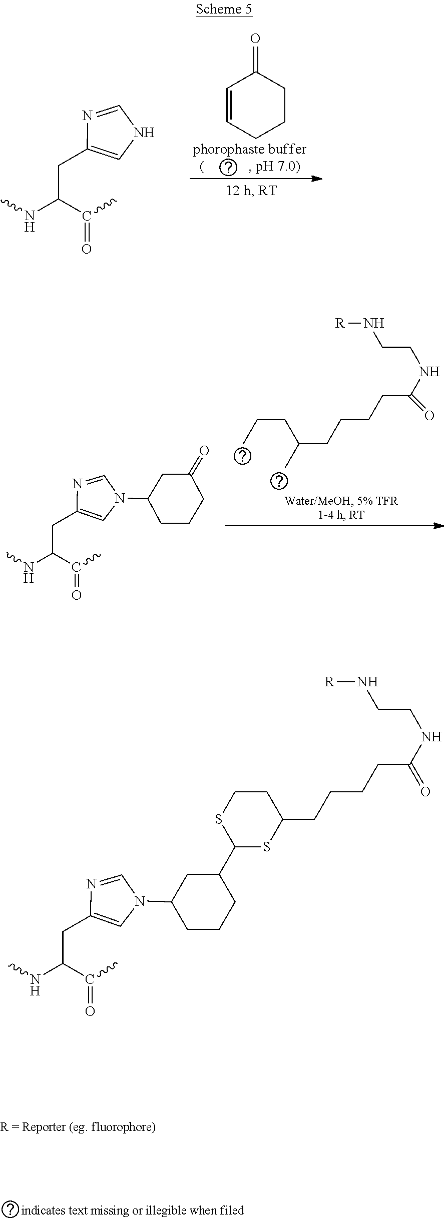

Scheme 6 provides an example of an arginine labeling mechanism. An arginine guanidinium can be acylated (e.g., labeled with an NHS ester with the aid of Barton's base). This example reaction may comprise cross-reactivity with primary amines (e.g., N-terminus, lysine) or thiols (e.g., cysteine), and thus may be performed lysine, cysteine, and/or N-terminal amine coupling steps.

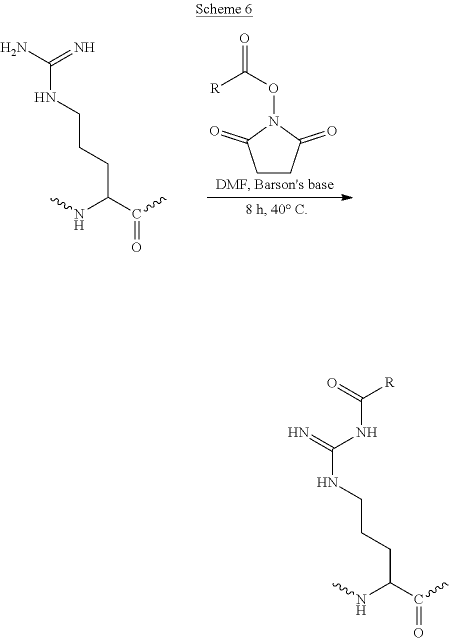

Methionine comprises a relatively low nucleophilicity and/or can often be selectively labeled by a redox based scheme utilizing an oxaziridine group configured to react with a methionine thioether (Scheme 7). The reagent may selectively label methionine over cysteine. The bond formed between the oxaziridine group and/or the methionine may be stable to reducing agents such as TCEP.

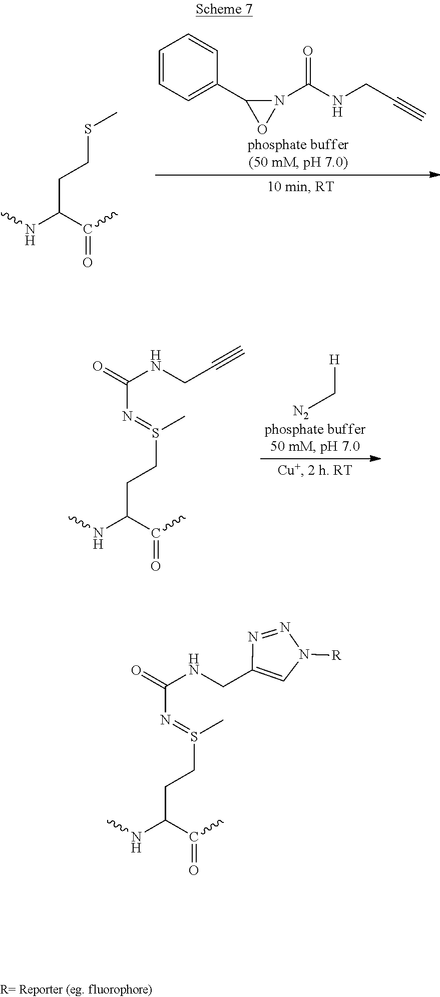

Scheme 8 provides an example of a tryptophan labeling scheme. A tryptophan indole may couple to a diazopropanoate ester, yielding a tertiary amine derivatized tryptophan. The coupling may be metal-catalyst mediated, for example, by a dirhodamine(II) tetraacetate complex. The catalyst may enhance selectivity for tryptophan over other amino acid types.

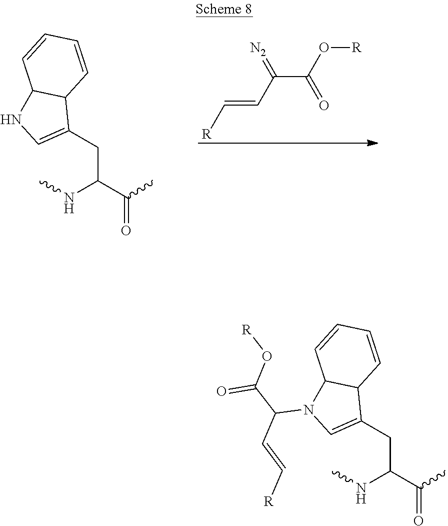

Phosphorylated amino acids such as phosphoserine, phosphotyrosine, or phosphothreonine may also be selectively labeled. Such a labeling method may distinguish between types of phosphorylated amino acids. For example, Scheme 9 below provides a phosphoryl beta-elimination followed by a label conjugate addition (e.g., a Michael acceptor reaction) step for selectively labeling of phosphoserine (pSer) and/or phosphothreonine (pThr) over other phosphorylated amino acids, such as phosphotyrosine (pTyr). Following such a step, a subsequent pan-phospho labeling method may be implemented to label remaining phosphoryl groups.

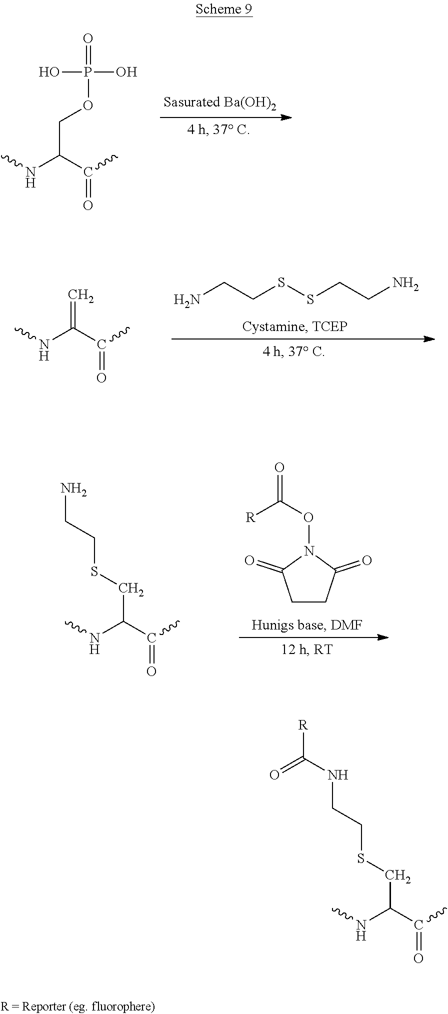

### Peptide Degradation

The present disclosure provides a range of chemical and/or enzymatic techniques for mild and/or sequential protein degradation. Degradation can be utilized in a range of peptide sequencing and/or analysis methods, for example, to determine the order or identity of particular amino acids in a fluorosequencing assay. A peptide or protein may be iteratively subjected to cleavage conditions to determine the sequence of at least a portion of the peptide sequence. The entire sequence of a peptide may be determined using the methods and/or compositions described herein. Controlled amino acid removal (e.g., N- or C-terminal amino acid removal) may be carried out through a variety of techniques including, for example, Edman degradation, organophosphate degradation, or proteolytic cleavage. In some aspects, Edman degradation may be used to remove a single terminal amino acid residue from a peptide N- or C-terminus. In some instances, the N-terminal amino acid residue of a peptide may be selectively removed. A chemical or enzymatic technique for removing a terminal amino acid may remove a defined number of (e.g., exactly one, exactly two, at most two) amino acids. Accordingly, a method for analyzing a peptide may comprise successive degradation and/or analysis steps, such that the removal of a defined number of amino acids from an N-terminus or C-terminus per step provides position and/or sequence specific amino acid identifications during analysis. A chemical or enzymatic technique for removing a terminal amino acid may cleave a peptide at a defined location (e.g., only in between two alanine residues, or only at the peptide bond connecting an N-terminal amino acid to the remainder of a peptide).

An Edman degradation method may comprise chemically functionalizing a peptide N-terminus or C-terminus (e.g., to form a thiourea or a guanidinium derivative of an N-terminal amine), and/or then contacting the functionalized terminal amino acid with a reagent (e.g., a hydrazine), a condition (e.g., a high or low pH or temperature), or an enzyme (e.g., an Edmanase with specificity for the functionalized terminal amino acid) to remove the functionalized terminal amino acid.

A deactivated phosphate or phosphonate may be used for peptide cleavage. Such a method may utilize an acid to remove a functionalized amino acid. The deactivated phosphate or phosphonate may be a dihalophosphate ester. In other embodiments, the techniques involve using an enzyme to remove the terminal amino acid residue, such as, for example, an exopeptidase or an Edmanase. For example, a method may comprise derivatizing an N-terminal amino acid of a peptide with a deactivated phosphate and/or contacting the peptide with an Edmanase enzyme with cleavage activity toward phosphate-functionalized N-terminal amino acids.

A cleavage method (e.g., a cleavage method implemented within a sequencing method) may comprise enzymatic cleavage. The cleavage method may comprise the use of a single protease, a series of proteases (e.g., provided in a specific order), or a combination of proteases. A cleavage method may comprise decoupling a peptide barcode from a molecule (e.g., a peptide or protein). For example, a peptide barcode may comprise a cleavable linker comprising a cleavage site recognized by a protease listed in TABLE 1. In such cases, the sequence of the cleavage site may be present in the cleavable linker and/or absent in the peptide barcode. A cleavage method may comprise fragmenting a peptide barcode (e.g., cleaving an internal peptide bond prior to peptide barcode sequencing).

TABLE 1
Example Proteases

| Protease | Cleavage Site |
|---|---|
| Carboxypeptidase A | C-terminal exopeptidase |
| Carboxypeptidase B | C-terminal exopeptidase; specific for lysine or arginine |
| Carboxypeptidase P | C-terminal exopeptidase |
| Carboxypeptidase Y | C-terminal exopeptidase |
| Cathepsin C | N-terminal exopeptidase; removes N-terminal dipeptide (except |
| | when N-terminal amino acid is lysine or arginine, or when 2^{nd} or |
| | 3^{rd} amino acid from N-terminal is proline) |
| Chymotrypsin | C-terminal exopeptidase; specific for phenylalanine, tryptophan, |
| | and/or tyrosine |
| Clostripain | Arginine |
| Elastase | Alanine, Valine, Serine, Glycine, Leucine, or Isoleucine |
| Endoproteinase Arg-C | Arginine |
| Endoproteinase Glu-C | Glutamic Acid |
| Endoproteinase Lys-C | Lysine |
| Glutamyl endopeptidase | Glutamic acid |
| Kallikrein (Plasma) | Lysine or Arginine |
| Papain | Lysine or Arginine followed by a hydrophobic residue |
| Pepsin | Leucine, Phenylalanine, Tryptophan or Tyrosine |
| Proteinase K | Aliphatic and/or aromatic amino acids |
| Subtilisin | Hydrophobic amino acids |
| TEV Protease | Specific for the sequences Glutamic acid-Asparagine-Leucine- |
| | Tyrosine-Phenylalanine-Glutamine-Glycine (SEQ ID NO: 4) |
| | and/or Glutamic acid-Asparagine-Leucine-Tyrosine-Phenylalanine- |
| | Glutamine-Serine (SEQ ID NO: 5); cleaves between Glutamine- |
| | Glycine or Glutamine-Serine |
| Thermolysin | Isoleucine, Methionine, Phenylalanine, Tryptophan, Tyrosine, or |
| | Valine |
| Trypsin | Lysine or Arginine |

Peptide cleavage may comprise chemical cleavage. Examples of chemical cleavage reagents consistent with the present disclosure include cyanogen bromide, BNPS-skatole, formic acid, hydroxylamine, and/or 2-nitro-5-thiocyanobenzoic acid. A peptide barcode may comprise a chemically cleavable moiety, such as a disulfide. A peptide barcode may be coupled to a molecule by a linker which comprises a chemically cleavable moiety. A peptide barcode may be coupled to a molecule by a chemically cleavable bond. A cleavage method may comprise a combination (e.g., parallel or sequential use) of chemical and/or enzymatic cleavage reagents. A cleavage method may comprise activating (e.g., functionalizing) an amino acid for chemical or enzymatic cleavage. For example, a method may comprise derivatizing an N-terminal amino acid residue of a peptide, and/or then contacting the peptide with an Edmanase enzyme configured to remove the derivatized N-terminal amino acid residue.

Peptide cleavage conditions may be achieved with a solvent. The solvent may be an aqueous solvent, an organic solvent, or a combination or mixture thereof. The solvent may be an organic solvent. The organic solvent may comprise a miscibility with water. The organic solvent may be anhydrous. The solvent may be a non-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethyl sulfoxide (DMSO), etc.), or a polar protic solvent (e.g., isopropanol (IPA), ethanol, methanol, acetic acid, water, etc.). The solvent may be DMF. The solvent may be a C₁-C₁₂ haloalkane. The C₁-C₁₂ haloalkane may be DCM. The solvent may be a mixture of two or more solvents. The mixture of two or more solvents may be a mixture of a polar aprotic solvent and/or a C₁-C₁₂ haloalkane. The mixture of two or more solvents may be a mixture of DMF and/or DCM. The mixture of solvents may be any combination thereof.

A degradation process may comprise a plurality of steps. For example, a method may comprise an initial step for derivatizing a terminal amino acid of a peptide, and/or a subsequent step for cleaving the derivatized terminal amino acid from the peptide. One such method comprises organophosphorus compound-mediated N-terminal functionalization and/or removal, and thus provides an alternative to the isothiocyanate (e.g., phenyl isothiocyanate) based processes of some Edman degradation schemes.

An organophosphate-based degradation scheme may comprise dissolving a peptide (e.g., a protein) in an organic solvent or organic solvent mixture (e.g., a mixture of DCM and/or DMF) in the presence of an organic base (e.g., triethylamine, N,N-diisopropylethylamine (DIPEA), 1,8-diazabicyclo[5.4.0]undec-7-ene (DBU), pyridine, 1,5-diazabicyclo(4.3.0)non-5-ene, 2,6-di-tert-butylpyridine, imidazole, histidine, sodium carbonate, etc.). The peptide may then be contacted with at least one organophosphorus compound. The cleavage of the peptide N-terminus may be initiated through the addition of a weak acid (e.g., formic acid in water). The cleavage of the peptide N-terminus may also be initiated with water. The resulting products may include the terminal amino acid of the peptide released from the peptide as a phosphoramide and/or the peptide that is shortened by the terminal amino acid residue, which comprises a free N-terminus that can be used to perform a subsequent cleavage reaction.

A cleavage method may comprise digesting a peptide to generate fragments of a desired average length. The cleavage method may generate peptides (e.g., by acting upon a complex mixture of peptides, such as cell lysate) with an average length of at least about 5 amino acids, at least about 8 amino acids, at least about 10 amino acids, at least about 12 amino acids, at least about 15 amino acids, at least about 20 amino acids, at least about 25 amino acids, at least about 30 amino acids, at least about 40 amino acids, or at least about 50 amino acids. The cleavage method may generate peptides with an average length of about 50 amino acids, about 40 amino acids, about 30 amino acids, about 25 amino acids, about 20 amino acids, about 15 amino acids, about 12 amino acids, about 10 amino acids, about 8 amino acids, or about 5 amino acids. The cleavage method may generate peptides with an average length of at most about 50 amino acids, at most about 40 amino acids, at most about 30 amino acids, at most about 25 amino acids, at most about 20 amino acids, at most about 15 amino acids, at most about 12 amino acids, at most about 10 amino acids, at most about 8 amino acids, or at most about 5 amino acids. The cleavage method may generate peptide fragments with an average length of between 5 and 20 amino acids, between 5 and 30 amino acids, between 10 and 20 amino acids, between 10 and 30 amino acids, between 12 and 18 amino acids, between 15 and 30 amino acids, between 20 and 40 amino acids, or between 30 and 50 amino acids.

A reaction mixture may comprise a stoichiometric or an excess concentration of a cleavage compound (e.g., relative to the concentration of peptides to be cleaved). The reaction mixture may comprise at least about 0.001% volume/volume (v/v), at least about 0.01% v/v, at least about 0.1% v/v, at least about 1% v/v, at least about 5% v/v, at least about 10% v/v, at least about 15% v/v, at least about 20% v/v, at least about 30% v/v, at least about 40% v/v, at least about 50% v/v, or more of the cleavage compound. The reaction mixture may comprise about 50% v/v, about 40% v/v, about 30% v/v, about 20% v/v, about 15% v/v, about 10% v/v, about 5% v/v, about 1% v/v, about 0.1% v/v, about 0.01% v/v, about 0.001% v/v, or less of the cleavage compound. The reaction mixture may comprise at most about 50% v/v, at most about 40% v/v, at most about 30% v/v, at most about 20% v/v, at most about 15% v/v, at most about 10% v/v, at most about 5% v/v, at most about 1% v/v, at most about 0.1% v/v, at most about 0.01% v/v, at most about 0.001% v/v, or less of the cleavage compound. The reaction mixture may comprise from about 0.1% v/v to about 20% v/v, about 0.5% v/v to about 10% v/v, or about 1% v/v to about 10% v/v of the cleavage compound.

The reaction mixture may comprise about 5% v/v of the cleavage compound. The reaction may be performed at a temperature of at least about 0° C., at least about 5° C., at least about 10° C., at least about 15° C., at least about 20° C., at least about 25° C., at least about 30° C., at least about 40° C., at least about 50° C., at least about 60° C., at least about 70° C., at least about 80° C., or at least about 90° C. The reaction may be performed at a temperature of about 90° C., about 80° C., about 70° C., about 60° C., about 50° C., about 40° C., about 30° C., about 25° C., about 20° C., about 15° C., about 10° C., about 5° C., about 0° C., or less. The reaction may be performed at a temperature of at most about 90° C., at most about 80° C., at most about 70° C., at most about 60° C., at most about 50° C., at most about 40° C., at most about 30° C., at most about 25° C., at most about 20° C., at most about 15° C., at most about 10° C., at most about 5° C., at most about 0° C., or less. The reaction may be performed at a temperature from about 0° C. to about 70° C., about 10° C. to about 50° C., about 20° C. to about 40° C., or about 20° C. to about 30° C. The reaction may be performed at a temperature above room temperature (e.g., about 22° C. to about 27° C.). The reaction may be performed at room temperature. The reaction may be performed at close to 0° C. or below 0° C. (e.g., in the presence of an antifreeze).

The peptide and the cleavage compound may be mixed or incubated for at least about 1 minute, at least about 5 minutes, at least about 10 minutes, at least about 20 minutes, at least about 30 minutes, at least about 40 minutes, at least about 50 minutes, at least about 60 minutes, at least about 2 hours, at least about 3 hours, at least about 4 hours, at least about 6 hours, at least about 8 hours, at least about 10 hours, at least about 12 hours, at least about 16 hours, at least about 20 hours, at least about 24 hours, or more. The peptide and the cleavage compound may be mixed or incubated for about 1 minute, about 5 minutes, about 10 minutes, about 20 minutes, about 30 minutes, about 40 minutes, about 50 minutes, about 60 minutes, about 2 hours, about 3 hours, about 4 hours, about 6 hours, about 8 hours, about 10 hours, about 12 hours, about 16 hours, about 20 hours, or about 24 hours. The peptide and the cleavage compound may be mixed or incubated for at most about 24 hours, at most about 20 hours, at most about 16 hours, at most about 12 hours, at most about 10 hours, at most about 8 hours, at most about 6 hours, at most about 4 hours, at most about 3 hours, at most about 2 hours, at most about 1 hour, at most about 50 minutes, at most about 40 minutes, at most about 30 minutes, at most about 20 minutes, at most about 10 minutes, at most about 5 minutes, at most about 1 minute, or less. The peptide and the cleavage compound may be mixed or incubated from about 1 minute to about 5 minutes, from about 5 minutes to about 10 minutes, from about 10 minutes to about 20 minutes, from about 20 minutes to about 30 minutes, from about 30 minutes to about 40 minutes, from about 40 minutes to about 50 minutes, from about 50 minutes to about 60 minutes, from about 60 minutes to about 3 hours, from about 3 hours to about 6 hours, from about 6 hours to about 12 hours, or from about 12 hours to about 24 hours.

### Information Storage

Computer data storage is a technology that has computer components and/or recording media used to retain data electronically. The most commonly used data storage technologies are semiconductor, magnetic, and/or optical. Data may be stored in data storage media, which data in a data storage device. A modern digital computer represents data using the binary numeral system. Text, numbers, pictures, audio, and/or nearly any other form of information can be converted into a string of bits, or binary digits, each of which has a value of 1 or 0. The most common unit of storage is the byte, equal to 8 bits. A piece of information can be handled by any computer or device whose storage space is large enough to accommodate the binary representation of the piece of information, or simply data.

While there are systems and/or methods presently available to store information electronically, recognized herein are various issues with such methods. Current systems and/or methods may not be capable of meeting the ever growing need for increased storage. As digital information continues to accumulate, higher density and/or longer-term storage solutions may be necessary, and/or current methods for storing information may not be capable of meeting the demand for higher density and/or longer-term storage.

Recognized herein is the need for improved methods and/or systems of storing data, accessing data and/or performing computations. Polypeptide-based data storage is an alternative to current systems and/or methods presently available to store data electronically. Polypeptide computing is a form of computing that uses polypeptides, biochemistry and/or molecular biology to store data, access data and/or perform computations. One potential advantage of polypeptide computing is that, similar to parallel computing, it can try many different possibilities at once owing to having many different amino acid options for polypeptides. In some embodiments, the devices and/or methods of the present disclosure have individually addressable arrays that can be used to perform computation using polypeptide molecules.

The present disclosure provides devices, systems and/or methods that employ the use of polypeptide sequences for data storage and/or computing. The systems and/or methods described herein have an array of sites referred to as pixels at which polypeptides can be synthesized, degraded, sequenced, attached, and/or detached. The pixels can be independently addressed, that is, each site can perform any one of polypeptide synthesis, degradation, sequencing, attachment, and/or detachment, irrespective of such actions being performed at any other site of the array. In some cases, an electrical field can be formed around each pixel to attract molecules to or repel molecules from the vicinity of the pixel. The present disclosure provides systems and/or methods for polypeptide-based computing that can be performed by the independent actions of an array of a large number of pixels (e.g., at least about 100, 1000, 10000, 50000, 100000, 500000, 1000000, 5000000, or 10000000 pixels).

Disclosed herein is a method comprising: (a) providing a polypeptide immobilized to a support, wherein the polypeptide comprises at least one labeled internal amino acid, and wherein the polypeptide encodes data; (b) detecting at least one signal or signal change from the polypeptide immobilized to the support to identify at least a portion of a sequence of the polypeptide; and/or (c) subjecting the polypeptide to conditions sufficient to remove at least one amino acid from the polypeptide. In some embodiments, the data are text. In some embodiments, the data are an image. In some embodiments, the data are numerical data. In some embodiments, the data are multimedia.

Data may be electronically encoded by assigning a bit pattern to each character, digit, or multimedia object. Many standards exist for encoding (e.g., character encodings like ASCII, image encodings like JPEG, video encodings like MPEG-4). Polypeptide computing is a form of computing that uses polypeptides, biochemistry and/or molecular biology to store data, access data and/or perform computations. One of potential advantage of polypeptide computing is that, similar to parallel computing, it can try many different possibilities at once owing to having many different amino acids within polypeptides.

Aspects of the present disclosure provide peptide barcodes for information storage. Many data storage systems offer fundamentally limited data storage densities and/or stabilities. For example, DNA-based information storage and/or magnetic memory storage can rapidly lose stored information when stored at room temperature, while silicon transistor-based memory storage systems are beginning to reach fundamental density limits imposed by quantum tunneling mechanisms. The peptide barcode storage systems of the present disclosure may be configured for dense and/or stable memory storage by retaining information within sequences comprising stable peptide bonds.

In some embodiments, the at least one labeled internal amino acid comprises a plurality of amino acid specific labels. In some embodiments, the amino acid specific labels comprise a methionine specific label, an arginine specific label, a histidine specific label, a tyrosine specific label, a carboxylic acid-containing amino acid specific label, a lysine specific label, a cysteine specific label, or any combination thereof. In some embodiments, the at least one labeled internal amino acid comprises an optically detectable label. In some embodiments, the at least one amino acid is removed from an N-terminus of the polypeptide.

In some embodiments, subsequent to (c), the at least one labeled internal amino acid becomes a labeled terminal amino acid. In some embodiments, the at least one labeled internal amino acid is from a plurality of labeled amino acids, and wherein the at least one signal or signal change comprises a collective signal from the plurality of labeled amino acids. In some embodiments, the plurality of labeled amino acids comprise amino acids with different labels. In some embodiments, the different labels generate signals with different signal patterns. In some embodiments, the at least one labeled internal amino acid comprises one or more members selected from the group consisting of lysine, glutamate, and aspartate. In some embodiments, the at least one labeled internal amino acid comprises an amino acid having a dye coupled thereto, which dye generates the at least one signal or signal change. In some embodiments, the at least one signal or signal change is an optical signal. In some embodiments, the at least one signal or signal change comprises a plurality of signals of different intensities. In some embodiments, the at least one signal or signal change comprises a plurality of signals of different frequencies or frequency ranges.

A peptide barcode (e.g., in the form of an amino acid sequence or composition) may be coupled to a substrate. The substrate may comprise an array of peptide barcodes coupled to spatially discrete locations on the substrate. For such a substrate, a peptide location may comprise additional information, for example, the locations of a plurality peptide barcodes of an array may denote an order in which the barcodes are intended to be read. A method for retrieving information may comprise coupling a peptide barcode to an array, for example by non-covalent coupling to a capture moiety (e.g., an antibody or a peptide nanopore structure) or by covalent coupling to an array-based linker. Conversely, a peptide barcode or a plurality of peptide barcodes may be stored in solution, frozen, lyophilized, or solid (e.g., powder) form.

In some embodiments, the system has individually addressable pixels where the data readout associated with each pixel may be accessed. As reactions of interest occur in each pixel, the data associated with each individual pixel may be accessed. For example, in the case of polypeptide sequencing, the data associated with the detection of a polypeptide and/or amino acid incorporation event may be accessed for the individual pixel where the incorporation event is occurring. This access may occur in real-time and/or there may be data readout for the particular pixel of interest as the reaction is happening and/or as the data is being generated. In other embodiments, the data may be accessed sometime after the data has been generated and/or sometime after the reaction of interest has occurred.

In some embodiments, the system may be used in conjunction with carrier particles, such as beads. In an embodiment, the beads may be magnetic and may bind to one or more magnets associated with individual pixels. In other embodiments, the system may not use carrier particles, but may bind biological and/or chemical targets of interest to each pixel in an alternate configuration. For example, the targets may be bound through a biotin-streptavidin bond, or contained in wells in the substrate.

A plurality of peptide barcodes may be disposed within a single molecule. For example, an information storage system may comprise a plurality of peptide barcodes disposed within a single peptide molecule and/or optionally coupled by cleavable linkers (e.g., protease recognition sites), such that the plurality of peptide barcodes may be stored as a single peptide or protein. The plurality of peptide barcodes may comprise or may be coupled to peptide sequences which impart a degree of secondary, tertiary, or quaternary structure. Such a system may enable particularly high-density information storage. For example, a peptide comprising 10⁵ amino acids may fold into a sphere-like particle with a diameter of less than 30 nm, corresponding to an information storage density of greater than 50 kilobytes per cubic nanometer (assuming 20 amino acid types and/or that each amino acid is identifiable).

Owing to the small size of amino acids, a peptide barcode may comprise a high degree of information density. A folded peptide barcode comprising amino acids selected from a set of twenty proteinogenic amino acids may comprise over 86 bits of information in a volume of less than about 3 nm³, or about 30 bits per nm³, comparing favorably to the leading solid state storage devices, which often provide less than 0.1 bits per nm². A peptide barcode may provide an information storage density of from about 0.01 bits per nm³ to about 0.02 bits per nm³, from about 0.02 bits per nm³ to about 0.05 bits per nm³, from about 0.05 bits per nm³ to about 0.1 bits per nm³, from about 0.1 bits per nm³ to about 0.25 bits per nm³, from about 0.25 bits per nm³ to about 0.5 bits per nm³, from about 0.5 bits per nm³ to about 1 bit per nm³, from about 1 bit per nm³ to about 2 bits per nm³, from about 2 bits per nm³ to about 3 bits per nm³, from about 3 bits per nm³ to about 4 bits per nm³, from about 4 bits per nm³ to about 5 bits per nm³, from about 5 bits per nm³ to about 6 bits per nm³, from about 6 bits per nm³ to about 8 bits per nm³, from about 8 bits per nm³ to about 10 bits per nm³, from about 10 bits per nm³ to about 15 bits per nm³, from about 15 bits per nm³ to about 20 bits per nm³, from about 20 bits per nm³ to about 25 bits per nm³, or from about 25 bits per nm³ to about 30 bits per nm³.

A peptide barcode may provide an information storage density of at least about 30 bits per nm³, at least about 25 bits per nm³, at least about 20 bits per nm³, at least about 15 bits per nm³, at least about 10 bits per nm³, at least about 8 bits per nm³, at least about 6 bits per nm³, at least about 5 bits per nm³, at least about 4 bits per nm³, at least about 3 bits per nm³, at least about 2 bits per nm³, at least about 1 bit per nm³, at least about 0.5 bits per nm³, at least about 0.25 bits per nm³, at least about 0.1 bits per nm³, at least about 0.05 bits per nm³, at least about 0.02 bits per nm³, or at least about 0.01 bits per nm³. A peptide barcode may provide an information storage density of about 30 bits per nm³, about 25 bits per nm³, about 20 bits per nm³, about 15 bits per nm³, about 10 bits per nm³, about 8 bits per nm³, about 6 bits per nm³, about 5 bits per nm³, about 4 bits per nm³, about 3 bits per nm³, about 2 bits per nm³, about 1 bit per nm³, about 0.5 bits per nm³, at least 0.25 about bits per nm³, about 0.1 bits per nm³, about 0.05 bits per nm³, about 0.02 bits per nm³, or about 0.01 bits per nm³. A peptide barcode may provide an information storage density of at most about 30 bits per nm³, at most about 25 bits per nm³, at most about 20 bits per nm³, at most about 15 bits per nm³, at most about 10 bits per nm³, at most about 8 bits per nm³, at most about 6 bits per nm³, at most about 5 bits per nm³, at most about 4 bits per nm³, at most about 3 bits per nm³, at most about 2 bits per nm³, at most about 1 bit per nm³, at most about 0.5 bits per nm³, at least 0.25 about bits per nm³, at most about 0.1 bits per nm³, at most about 0.05 bits per nm³, at most about 0.02 bits per nm³, or at most about 0.01 bits per nm³.

An information storage system of the present disclosure may comprise a storage density of from about 10³ to about 10⁴, from about 10⁴ to about 10⁵, from about 10⁵ to about 10⁶, from about 10⁶ to about 10⁷, from about 10⁷ to about 10⁸, from about 10⁸ to about 10⁹, from about 10⁹ to about 10¹⁰, from about 10¹⁰ to about 10¹¹, from about 10¹¹ to about 10¹², from about 10¹² to about 10¹⁵, from about 10¹⁵ to about 10²⁰, from about 10²⁰ to about 10²⁵, or from about 10²⁵ to about 10³⁰ bytes/cm³.

An information storage system of the present disclosure may comprise a storage density of at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵, at least about 10¹⁶, at least about 10¹⁷, at least about 10¹⁸, at least about 10¹⁹, at least about 10²⁰, at least about 10²¹, at least about 10²², at least about 10²³, at least about 10²⁴, at least about 10²⁵, at least about 10²⁶, at least about 10²⁷, at least about 10²⁸, at least about 10²⁹, or at least about 10³⁰ bytes/cm³. An information storage system of the present disclosure may comprise a storage density of about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰, about 10²¹, about 10²², about 10²³, about 10²⁴, about 10²⁵, about 10²⁶, about 10²⁷, about 10²⁸, about 10²⁹, or about 10³⁰ bytes/cm³.

An information storage system of the present disclosure may comprise a storage density of at most about 10³, at most about 10⁴, at most about 10⁵, at most about 10⁶, at most about 10⁷, at most about 10⁸, at most about 10⁹, at most about 10¹⁰, at most about 10¹¹, at most about 10¹², at most about 10¹³, at most about 10¹⁴, at most about 10¹⁵, at most about 10¹⁶, at most about 10¹⁷, at most about 10¹⁸, at most about 10¹⁹, at most about 10²⁰, at most about 10²¹, at most about 10²², at most about 10²³, at most about 10²⁴, at most about 10²⁵, at most about 10²⁶, at most about 10²⁷, at most about 10²⁸, at most about 10²⁹, or at most about 10³⁰ bytes/cm³.

In some embodiments, the method further comprises cleaving the polypeptide from the support. In some embodiments, at least one amino acid is removed from the polypeptide by a degradation reaction. In some embodiments, the degradation reaction is Edman degradation. In some embodiments, the polypeptide is a protein. In some embodiments, the polypeptide is part of a protein. In some embodiments, the at least one signal or signal change is detected with an optical detector having single-molecule sensitivity. In some embodiments, the method further comprises processing the at least the portion of the sequence against a reference sequence to identify the polypeptide or a protein from which the polypeptide is derived. In some embodiments, the method further comprises, subsequent to (c), (i) identifying the at least the portion of the sequence of the polypeptide to identify the polypeptide, and/or (ii) using the polypeptide identified in (i) to quantify the polypeptide or a protein from which the polypeptide was derived.

An aspect of the present disclosure provides a method for accessing data. The method can comprise providing an array of individually addressable sites, where a given site of the array has a polypeptide molecule with a sequence of amino acid subunits that corresponds to bits encoding at least one computer-executable directive for storing data. The method can include, at the given site, identifying the sequence of amino acid using sequencing by degradation. The method can use a computer processor to identify the bits from the sequence of amino acid subunits and/or generate the data from the bits.

Information may be extracted from a peptide barcode through a variety of methods. A peptide barcode may be analyzed optically (e.g., fluorometrically), chemically, electrochemically (e.g., using nanopores), by mass spectrometry, compositionally (e.g., by elemental analysis), chromatographically, electrophoretically, or any combination thereof.

In such a method, according to some embodiments, there may be a substrate having a plurality of locations, or pixels, for containing biological matter. The biological matter can for instance be a labeled polypeptide. Labeled polypeptides can be delivered to specific pixels on the substrate of a single chip and/or these pixels can also be referred to as “nano-reactors.” Identifying the polypeptide sequence of the amino acid subunits can comprise sequencing the amino acid molecule. In some cases, the sequencing comprises performing sequencing by degradation as described herein. The sequence of amino acid subunits can be stored in computer memory. In some cases, the method further comprises storing the data in computer memory.

Information may be retrieved from a peptide barcode through sequencing. Such a method may comprise fluorosequencing. For example, the method may comprise coupling the peptide barcode to a support (e.g., a support comprising an array of peptides), labeling at least a subset of amino acids or amino acid sequences of the peptide barcode, and/or detecting labels coupled to the peptide barcode. Optionally, the detecting may comprise peptide barcode degradation. For example, a fluorosequencing method may comprise iteratively performing detection and/or terminal amino acid removal steps, such that each detecting round provides information regarding the previous terminal amino acid. A sequencing method may comprise antibody-based peptide barcode analysis or terminal amino acid binding agent-based peptide barcode analysis. For example, a sequencing method may employ a plurality of uniquely identifiable N-terminal amino binding proteins configured to couple to single amino acid types at peptide N- or C-terminals. A sequencing method may comprise mass spectrometric analysis.

In some embodiments, in (a), less than all amino acids of the polypeptide are labeled. In some embodiments, the method further comprises (i) repeating (b) and/or (c) to detect at least one additional signal or signal change from the polypeptide immobilized to the support and/or (ii) using the at least one signal or signal change and/or the at least one additional signal or signal change to identify the at least the portion of the sequence. In some embodiments, the detecting identifies a sequence of the polypeptide. In some embodiments, the detecting is performed at a read rate of at least 36 bits/s. In some embodiments, the detecting comprises fluorimetry. In some embodiments, the detecting comprises imaging.

In some embodiments, the method further comprises assigning the polypeptide a optically resolvable address. In some embodiments, the optically resolvable address comprises digital information. In some embodiments, the method further comprises comparing the portion of the sequence of the polypeptide against a database of known sequences. In some embodiments, the method further comprises, prior to (a), coupling the polypeptide to the support. In some embodiments, the method further comprises determining a physical property of the polypeptide. In some embodiments, the physical property is selected from the group consisting of isoelectric point, molecular weight, and/or hydrophobicity index. In some embodiments, the method further comprises, prior to (a), coupling the polypeptide to an array. In some embodiments, the method further comprises lyophilizing the array. In some embodiments, the array comprises an information storage density of at least 10⁷ bytes/cm³. In some embodiments, the array comprises an information storage density of at least 10³⁰ bytes/cm³.

In some embodiments, the polypeptides can comprise at least two distinct subunits, where a subset of the at least two distinct subunits corresponds to a 1 or 0. In some cases, a given site comprises a plurality of the amino acid molecules. The method can further comprise assembling generated data into a larger piece of data.

A sequencing method may comprise nanopore analysis. A nanopore sequencing method disclosed herein can provide peptide sequence information at the single molecule level. Nanopore sequencing involves passing single strands of biomolecules through a tiny protein channel (nanopore) embedded in an electrically resistant membrane. A voltage is applied across the nanopore to cause a stretch of the biomolecule to thread through the nanopore. A sequence of the biomolecule can be identified based on changes in ion current flowing through the nanopore that are associated with each monomeric unit of the biomolecule. In this manner, for example, individual amino acids of a peptide sequence can be identified. A nanopore sequencing method disclosed herein may employ a proteosome that controls the unfolding and/or linearized transport of proteins across the nanopore. Similar to fluorosequencing methods, nanopore sequencing methods may involve coupling amino acid labels to a peptide to be sequenced. A method consistent with the present disclosure may subject a peptide to nanopore sequencing and/or an additional form of analysis. For example, nanopore sequencing can be combined with machine learning techniques and/or reference peptide or proteome databases. A sequencing method may read peptide barcode data at a range of rates. A sequencing method may read information at a rate of from about 1 bit to about 5 bits, from about 5 bits to about 10 bits, from about 10 bits to about 20 bits, from about 20 bits to about 64 bits, from about 64 bits to about 128 bits, from about 128 bits to about 256 bits, from about 256 bits to about 512 bits, from about 512 bits to about 1 kilobits, from about 1 kilobits to about 5 kilobits, from about 5 kilobits to about 10 kilobits, from about 10 kilobits to about 32 kilobits, from about 32 kilobits to about 64 kilobits, from about 64 kilobits to about 128 kilobits, from about 128 kilobits to about 256 kilobits, from about 256 kilobits to about 512 kilobits, or from about 512 kilobits to about 1 megabit per second (bits/s).

A sequencing method may read peptide barcode data at a range of rates. A sequencing method may read information at a rate of at least about 1 bit, at least about 2 bits, at least about 3 bits, at least about 4 bits, at least about 5 bits, at least about 6 bits, at least about 7 bits, at least about 8 bits, at least about 9 bits, at least about 10 bits, at least about 12 bits, at least about 16 bits, at least about 20 bits, at least about 24 bits, at least about 28 bits, at least about 36 bits, at least about 64 bits, at least about 128 bits, at least about 256 bits, at least about 512 bits, at least about 1 kilobit, at least about 2 kilobits, at least about 4 kilobits, at least about 8 kilobits, at least about 16 kilobits, at least about 32 kilobits, at least about 64 kilobits, at least about 128 kilobits, at least about 256 kilobits, at least about 512 kilobits, or at least about 1 megabit per second (bits/s). A sequencing method may read information at a rate of about 1 bit, about 2 bits, about 3 bits, about 4 bits, about 5 bits, about 6 bits, about 7 bits, about 8 bits, about 9 bits, about 10 bits, about 12 bits, about 16 bits, about 20 bits, about 24 bits, about 28 bits, about 36 bits, about 64 bits, about 128 bits, about 256 bits, about 512 bits, about 1 kilobit, about 2 kilobits, about 4 kilobits, about 8 kilobits, about 16 kilobits, about 32 kilobits, about 64 kilobits, about 128 kilobits, about 256 kilobits, about 512 kilobits, or about 1 megabit per second (bits/s). A sequencing method may read information at a rate of at most about 1 bit, at most about 2 bits, at most about 3 bits, at most about 4 bits, at most about 5 bits, at most about 6 bits, at most about 7 bits, at most about 8 bits, at most about 9 bits, at most about 10 bits, at most about 12 bits, at most about 16 bits, at most about 20 bits, at most about 24 bits, at most about 28 bits, at most about 36 bits, at most about 64 bits, at most about 128 bits, at most about 256 bits, at most about 512 bits, at most about 1 kilobit, at most about 2 kilobits, at most about 4 kilobits, at most about 8 kilobits, at most about 16 kilobits, at most about 32 kilobits, at most about 64 kilobits, at most about 128 kilobits, at most about 256 kilobits, at most about 512 kilobits, or at most about 1 megabit per second (bits/s). For example, an array of peptides may be subjected to fluorosequencing, such that the rate of information retrieval from the array is a multiple of the rate of fluorosequencing and/or the number of peptide barcodes of the array.

The sequencing method may identify a subset of amino acids within a peptide barcode sequence. In such cases, the partial peptide barcode sequence may be filled in via comparison to a database. A peptide barcode or peptide barcode library may comprise a finite number of sequences, such that a partial sequence determined for the peptide barcode or for a peptide barcode of the peptide barcode library may be uniquely matched to a complete peptide sequence from the database. Accordingly, a relatively short peptide barcode sequence (e.g., a sequence comprising 216 bits of information) may denote extensive information (e.g., kilobits, megabits, gigabits, or greater) when matched against a database. Such a system may be used for secure data encryption. For example, a data-receiving party may comprise a unique peptide sequence database for interpreting partial sequence information from a library of peptide barcodes.

An aspect of the present disclosure provides a method for data storage. The method can comprise receiving bits encoding at least one computer-executable directive for storing data. The method can use a computer processor to generate a polypeptide sequence that encodes the data, where the polypeptide sequence comprises amino acid subunits that correspond to the bits. The method can include using an array of individually addressable polypeptide synthesis sites to generate a polypeptide molecule having an amino acid sequence at a first site of the array at the exclusion of generating an additional polypeptide molecule having the amino acid sequence at a second site of the array.

In some embodiments, the system may be used to store information, similar to a hard drive in a traditional computer. Amino acids (e.g., alanine (A), glycine (G), isoleucine (I), leucine (L), proline (P), valine (V), phenylalanine (F), tryptophan (W), tyrosine (Y), aspartic acid (D), glutamic acid (E), arginine (R), histidine (H), lysine (K), serine (S), threonine (T), cysteine (C), methionine (M), asparagine (N), and/or glutamine (Q)) and/or various combinations of these in different lengths can be used to “code” for information. In this manner, virtually any type of information can be “stored” within the polypeptide. In a further embodiment, a polypeptide can be considered to have up to twenty “bits” (e.g., the amino acids A, G, I, L, P, V, F, W, Y, D, E, R, H, K, S, T, C, M, N, and/or Q), versus a traditional computer transistor that only has two bits (the binary 0 and 1). Furthermore, polypeptide molecules can be three-dimensional (3D) and/or have directionality on the z-axis, where the distance between each layer is about 3 angstroms or less. These properties of polypeptides can allow for very dense storage.

In some embodiments, a peptide barcode can be used to store information using 2, 3, 4, 5, 6, 7, 8, 9, 10, 11, 12, 13, 14, 15, 16, 17, 18, 19, or 20 bits per amino acid. In some embodiments, each amino acid residue can correspond to a single “bit”. In some embodiments, each amino acid residue can correspond to a “bit” corresponding to a binary digit used in computer systems. In some embodiments, each amino acid residue can correspond to a sequence of bit, wherein each bit corresponds to a binary digit used in computer systems.

In some embodiments, a polypeptide can be used to store information using 3 bits per amino acid. In some embodiments, a polypeptide can be used to store information using 4 bits per amino acid. In some embodiments, a polypeptide can be used to store information using 5 bits per amino acid. In some embodiments, a polypeptide can be used to store information using 6 bits per amino acid. In some embodiments, a polypeptide can be used to store information using 7 bits per amino acid. In some embodiments, a polypeptide can be used to store information using 8 bits per amino acid. In some embodiments, a polypeptide can be used to store information using 9 bits per amino acid. In some embodiments, a polypeptide can be used to store information using 10 bits per amino acid. In some embodiments, a polypeptide can be used to store information using 11 bits per amino acid. In some embodiments, a polypeptide can be used to store information using 12 bits per amino acid. In some embodiments, a polypeptide can be used to store information using 13 bits per amino acid. In some embodiments, a polypeptide can be used to store information using 14 bits per amino acid. In some embodiments, a polypeptide can be used to store information using 15 bits per amino acid. In some embodiments, a polypeptide can be used to store information using 16 bits per amino acid. In some embodiments, a polypeptide can be used to store information using 17 bits per amino acid. In some embodiments, a polypeptide can be used to store information using 18 bits per amino acid. In some embodiments, a polypeptide can be used to store information using 19 bits per amino acid. In some embodiments, a polypeptide can be used to store information using 20 bits per amino acid. In some embodiments, a polypeptide can be used to store information using from about 2 bits to about 5 bits, from about 5 bits to about 10 bits, from about 10 bits to about 15 bits, or from about 15 bits to about 20 bits per amino acid. In some embodiments, a polypeptide can be used to store information using at least 2 bits, at least 3 bits, at least 4 bits, at least 5 bits, at least 6 bits, at least 7 bits, at least 8 bits, at least 9 bits, at least 10 bits, at least 11 bits, at least 12 bits, at least 13 bits, at least 14 bits, at least 15 bits, at least 16 bits, at least 17 bits, at least 18 bits, at least 19 bits, or at least 20 bits per amino acid. In some embodiments, a polypeptide can be used to store information using at most 2 bits, at most 3 bits, at most 4 bits, at most 5 bits, at most 6 bits, at most 7 bits, at most 8 bits, at most 9 bits, at most 10 bits, at most 11 bits, at most 12 bits, at most 13 bits, at most 14 bits, at most 15 bits, at most 16 bits, at most 17 bits, at most 18 bits, at most 19 bits, or at most 20 bits per amino acid.

The present disclosure provides an example of a code for translating a polypeptide sequence to numerical data. For example, the following values from 1-20 can be mapped to the following amino acids as shown in TABLE 2. In some embodiments, one amino acid can be designated as a “break”, indicating the end of a code and/or beginning of a new code. In some embodiments, one-to-one mapping of bit sequences to amino acids can be used to store 3 bits per amino acid, as shown in TABLE 3.

TABLE 2

| Amino acid | Number |
|---|---|
| Alanine (A) | 1 |
| Glycine (G) | 2 |
| Isoleucine (I) | 3 |
| Leucine (L) | 4 |
| Proline (P) | 5 |
| Valine (V) | 6 |
| Phenylalanine (F) | 7 |
| Tryptophan (W) | 8 |
| Tyrosine (Y) | 9 |
| Aspartic acid (D) | 10 |
| Glutamic acid (E) | 11 |
| Arginine (R) | 12 |
| Histidine (H) | 13 |
| Lysine (K) | 14 |
| Serine (S) | 15 |
| Threonine (T) | 16 |
| Cysteine (C) | 17 |
| Methionine (M) | 18 |
| Asparagine (N) | 19 |
| Glutamine (Q) | 20 |

TABLE 3

| Bit sequence | | 000 | 001 | 010 | 011 | 100 | 101 | 110 | 111 |
|---|---|---|---|---|---|---|---|---|---|
| Amino | Data | S | T | E | Y | A | V | L | F |
| acid | set A | | | | | | | | |
| | Data | Y | T | E | V | A | S | L | F |
| | set B | | | | | | | | |

In some embodiments, the system may also have the capability to allow for the synthesis of polypeptides, or allow for “polypeptide writing.” In some embodiments, the user may wish to create a particular polypeptide sequence and/or slight variations of a known polypeptide sequence. In some embodiments, a polypeptide with a known sequence may be located inside an individual pixel. The polypeptide may be held in a location in the pixel by a primer, a chemical bond, or a bead (e.g., magnetically attractable bead). In other embodiments, there may be a plurality of polypeptides in various locations in the pixels. The methods of the disclosure can further comprise removing the polypeptide from the array.

In some embodiments, the bits can encode a plurality of computer-executable directives. The data can be stored in computer memory. In some cases, the polypeptide sequence can be stored in computer memory. In some instances, the amino acid subunits can be selected from at least two distinct subunits, where a subset of the at least two distinct subunits corresponds to a 1 or 0.

In some cases, the polypeptide molecule can be generated on a reaction surface at the first site. The reaction surface can be a particle or a surface of a well at the first site. In some cases, the polypeptide molecule can be generated on the reaction surface via covalent coupling of an amino acid subunit or precursor thereof of the polypeptide molecule to the reaction surface. In some instances, the polypeptide molecule can be generated on the reaction surface via coupling of an amino acid subunit or precursor thereof of the polypeptide molecule to a linker coupled to the reaction surface. The linker can comprise another polypeptide or a chemical linker.

In some cases, the array can be substantially planar (e.g., deviates from a plane by no more than 0.1%, 0.5%, 1%, 5%, or 10% of the longest dimension of the array at any one point of the plane).

The basic operations of polypeptide synthesis, degradation, sequencing, attachment, and/or detachment can be combined to create any number of computational modules. A computational module can involve any combination of storing, writing and/or manipulating a polypeptide molecule according to a programmed algorithm.

### Sample Types

The methods described herein may comprise analyzing a biological sample. A biological sample may be derived from a subject (e.g., a patient or a participant in a study), from a tissue sample (e.g., an engineered tissue sample), from a cell culture (e.g., a human cell line or a bacterial colony), from a cell (e.g., a cell isolated during a single cell sorting assay), or a portion thereof (e.g., an organelle from a cell or an exosome from a blood sample). A biological sample may be synthetic, such as a composition of synthetic peptides. A sample may comprise a single species or a mixture of species. A biological sample may comprise biomaterial from a single organism, from a colony of genetically near-identical organisms, or from multiple organisms (e.g., enterocytes and/or microbiota from a human digestive tract). A biological sample may be fractionated (e.g., plasma separated from whole blood), filtered, or depleted (e.g., high abundance proteins such as albumin and/or ceruloplasmin removed from plasma).

A sample may comprise all or a subset of the biomolecules from the subject, tissue sample, cell culture, cell, or portion thereof. For example, a sample from a subject may comprise the majority of proteins present in that subject or may comprise a small subset of the proteins from that subject. A biological sample may comprise a bodily fluid such as cerebral spinal fluid (CSF), saliva, urine, tears, blood, plasma, serum, breast aspirate, prostate fluid, seminal fluid, stool, amniotic fluid, intraocular fluid, mucous, or any combination thereof. A biological sample may comprise a tissue culture, for example a tumor sample, or tissue from a kidney, liver, lung, pancreas, stomach, intestine, bladder, ovary, testis, skin, colorectal, breast, brain, esophagus, placenta, or prostate.

The biological sample may comprise a molecule whose presence or absence may be measured or identified. The biological sample may comprise a macromolecule, such as, for example, a polypeptide or a protein. The macromolecule may be isolated (e.g., separated from other components from which the macromolecule was sourced) or purified, such that the macromolecule comprises at least about 0.5%, at least about 1%, at least about 2%, at least about 3%, at least about 4%, at least about 5%, at least about 7.5%, at least about 10%, at least about 15%, at least about 20%, at least about 25%, at least about 30%, at least about 40%, at least about 50%, at least about 60%, at least about 70%, at least about 75%, at least about 80%, at least about 90%, at least about 95%, at least about 98%, or at least about 99% of a composition by weight (e.g., by dry weight or including solvent). The macromolecule may be isolated (e.g., separated from other components from which the macromolecule was sourced) or purified, such that the macromolecule comprises about 0.5%, about 1%, about 2%, about 3%, about 4%, about 5%, about 7.5%, about 10%, about 15%, about 20%, about 25%, about 30%, about 40%, about 50%, about 60%, about 70%, about 75%, about 80%, about 90%, about 95%, about 98%, or about 99% of a composition by weight (e.g., by dry weight or including solvent). The macromolecule may be isolated (e.g., separated from other components from which the macromolecule was sourced) or purified, such that the macromolecule comprises at most about 0.5%, at most about 1%, at most about 2%, at most about 3%, at most about 4%, at most about 5%, at most about 7.5%, at most about 10%, at most about 15%, at most about 20%, at most about 25%, at most about 30%, at most about 40%, at most about 50%, at most about 60%, at most about 70%, at most about 75%, at most about 80%, at most about 90%, at most about 95%, at most about 98%, or at most about 99% of a composition by weight (e.g., by dry weight or including solvent).

The biological sample may be complex, and/or may comprise a plurality of components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single type (e.g., peptides, nucleic acids, lipids, small molecules). A biological sample may comprise a plurality of peptides configured for a method of the present disclosure (e.g., digestion, C-terminal labeling, or fluorosequencing).

Methods consistent with the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for different analyses (e.g., CTC lysate and/or buffy coat proteins may be partitioned and/or separately analyzed) or pooled for common analysis. A biological species may be homogenized, fragmented, or lysed prior to analysis. In particular instances, a species or plurality of species from among the homogenate, fragmentation products, or lysate may be collected for analysis. For example, a method may comprise collecting circulating tumor cells during a liquid biopsy, optionally isolating individual circulating tumor cells, lysing the circulating tumor cells, isolating peptides from the resulting lysate, and/or analyzing the peptides by a fluorosequencing method of the present disclosure. A method may comprise capturing peptides from a sample using a C-terminal capture reagent, and/or analyzing the peptides (e.g., by a fluorosequencing method).

Methods consistent with the present disclosure may comprise nucleic acid analysis, such as sequencing, southern blot, or epigenetic analysis. Nucleic acid analysis may be performed in parallel with a second analytical method, such as a fluorosequencing method of the present disclosure. The nucleic acid and/or the subject of the second analytical method may be derived from the same subject or the same sample. For example, a method may comprise collecting cell free DNA and/or proteins from a human plasma sample, sequencing the cell free DNA (e.g., to identify a cancer marker), and/or performing proteomic analysis on the plasma proteins.

### Computer Systems

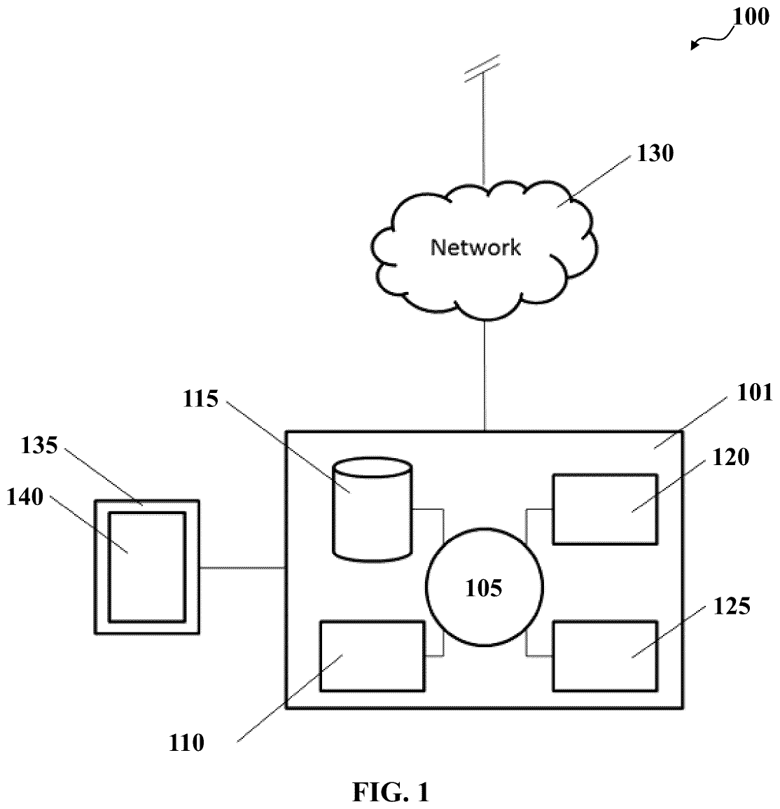

The present disclosure provides computer systems that are programmed to implement methods of the disclosure. FIG. 1 shows a computer system 101 that is programmed or otherwise configured to implement methods or parts of methods disclosed herein, including compiling, analyzing, and/or displaying data obtained through the present methods. The computer system 101 may regulate various aspects of the present disclosure, such as, for example, controlling cell partitioning and/or optical imaging devices. The computer system 101 may be an electronic device of a user or a computer system that is remotely located with respect to the electronic device. The electronic device may be a mobile electronic device.

The computer system 101 includes a central processing unit (CPU, also “processor” and/or “computer processor” herein) 105, which may be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and/or peripheral devices 125, such as cache, other memory, data storage, and/or electronic display adapters. The memory 110, storage unit 115, interface 120 and/or peripheral devices 125 are in communication with the CPU 105 through a communication bus (solid lines), such as a motherboard. The storage unit 115 may be a data storage unit (or data repository) for storing data. The computer system 101 may be operatively coupled to a computer network (“network”) 130 with the aid of the communication interface 120. The network 130 may be the Internet, an internet and/or extranet, or an intranet and/or extranet that is in communication with the Internet. The network 130 in some cases is a telecommunication and/or data network. The network 130 may include one or more computer servers, which may enable distributed computing, such as cloud computing. The network 130, in some cases with the aid of the computer system 101, may implement a peer-to-peer network, which may enable devices coupled to the computer system 101 to behave as a client or a server.

The CPU 105 may execute a sequence of machine-readable instructions, which may be embodied in a program or software. The instructions may be stored in a memory location, such as the memory 110. The instructions may be directed to the CPU 105, which may subsequently program or otherwise configure the CPU 105 to implement methods of the present disclosure. Examples of operations performed by the CPU 105 may include fetch, decode, execute, and/or writeback.

The CPU 105 may be part of a circuit, such as an integrated circuit. One or more other components of the system 101 may be included in the circuit. In some cases, the circuit is an application specific integrated circuit (ASIC).

The storage unit 115 may store files, such as drivers, libraries, and/or saved programs. The storage unit 115 may store user data, e.g., user preferences and/or user programs. The computer system 101 in some cases may include one or more additional data storage units that are external to the computer system 101, such as located on a remote server that is in communication with the computer system 101 through an intranet or the Internet.

The computer system 101 may communicate with one or more remote computer systems through the network 130. For instance, the computer system 101 may communicate with a remote computer system of a user (e.g., a fluorimeter or a cell sorting device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 101 via the network 130.

Methods as described herein may be implemented by way of machine (e.g., computer processor) executable code stored on an electronic storage location of the computer system 101, such as, for example, on the memory 110 or electronic storage unit 115. The machine executable or machine readable code may be provided in the form of software. During use, the code may be executed by the processor 105. In some cases, the code may be retrieved from the storage unit 115 and/or stored on the memory 110 for ready access by the processor 105. In some situations, the electronic storage unit 115 may be precluded, and/or machine-executable instructions are stored on memory 110.

The code may be pre-compiled and/or configured for use with a machine having a processer adapted to execute the code or may be compiled during runtime. The code may be supplied in a programming language that may be selected to enable the code to execute in a pre-compiled or as-compiled fashion.

Aspects of the systems and/or methods provided herein, such as the computer system 101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and/or the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and/or electromagnetic waves, such as used across physical interfaces between local devices, through wired and/or optical landline networks and/or over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and/or fiber optics, including the wires that comprise a bus within a computer system. Carrier-wave transmission media may take the form of electric or electromagnetic signals, or acoustic or light waves such as those generated during radio frequency (RF) and/or infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and/or EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 may include or be in communication with an electronic display 135 that comprises a user interface (UI) 140 for providing, for example, orders and/or options for controlling flow rates in a cell sorting device. Examples of UI's include, without limitation, a graphical user interface (GUI) and/or web-based user interface.

Methods and/or systems of the present disclosure may be implemented by way of one or more algorithms. An algorithm may be implemented by way of software upon execution by the central processing unit 105. The algorithm may, for example, determine a correlation using linear and/or quadratic discriminant analysis (LDA and/or QDA), Support Vector Machine (SVM), linear discriminant analysis (LDA), quadratic discriminant analysis (QDA), Naive Bayes, Random Forest, or any other suitable method.

### EXAMPLES

### Example 1: Multiplexing Samples for Protein Quantification

Multiplexing samples and using fluorosequencing as an analytical technique to measure levels of the target proteins removes biases in sample handling and processing. Expression levels of two target proteins present in the proteasomes for two different samples are quantified. The expression levels of two target proteins—PSME4 (Uniprot ID: Q14997) and PSMB10 (Uniprot ID: P40306)—are used to differentiate the immunogenic reaction of lung cancer cell lines.

Culturing cell lines and TNF treatment: Cell line A549 (ATCC, Cat #CCL-185) is cultured in DMEM media, supplemented with 10% Fetal bovine serum, 1% Penicillin and L-glutamine (2 mmol/L) at 37° C. and 5% CO₂. For activating the immune-response in the A549 cell-line, termed “A549-Ti”, TNF-alpha and IFN-gamma are added at concentrations of 20 ng/mL and 10 ng/mL, respectively for 24h after an overnight seeding.

Proteasomal immunoprecipitation and Protein amount: Cells are lysed in 25 mM HEPES, pH 7.5, 10% glycerol, 5 mM MgCl₂, 1 mM ATP and a 1:400 protease-inhibitor mixture (Calbiochem). The cells are homogenized through freeze-thaw cycles. The lysates are cleared by 30-min centrifugation at 10,000 rpm at 4° C. to remove cell debris. A pre-clearing step is performed by incubating the cells with Protein A/G conjugated Magnetic beads (Thermo) for 30 min at 4° C. 1 mg of pre-cleared cell lysates are then added to 20 μL of fresh beads along with 10 ug of antibody—anti-PSMA2 (Abcam). The mixture is incubated overnight at 4° C. with rotation. Following the incubation, the beads are washed thrice with 100 mM NaCl in PBS buffer. The immunoprecipitation of proteasome is confirmed using western blot, using antibodies for PSME4 (abcam) and PSMB10 (abeam). The proteins are eluted using a 0.1 M glycine buffer (pH 2.5). The proteins are then concentrated by speed-vac, and an approximate amount of 10 μg is aliquoted and dissolved in 200 μL of phosphate buffer (pH 8.5). The two samples are termed—“Ctrl” and “Ti treated”.

Construction of peptide-barcode for N-terminal protein ligation: Two different barcoded peptide sequences are prepared by conjugating the peptide barcode (Fmoc-KAKA-COOH (SEQ ID NO: 6) and Fmoc-KAAK—COOH (SEQ ID NO: 7), where K and A are single letter amino acid codes and Fmoc: 9-Fluorenylmethyl chloroformate) to peptide H₂N-DFSKL-Cam (SEQ ID NO: 8) ester, using solid-phase peptide synthesis. The resulting peptide-barcodes BC1 and BC2 with sequence—Fmoc-KAKADFSKL-Cam (SEQ ID NO: 9) ester and Fmoc-KAAKDFSKL-Cam (SEQ ID NO: 10) ester, respectively, are weighed and aliquoted to 1 mg (1 μmole) each.

Ligation of peptide-barcode to immunoprecipitated proteasomal proteins using Omniligase: Enzyme Omniligase is used to ligate barcoded-peptides to N-termini of proteins. 1 mg of BC1 is added to 200 μL of Ctrl sample, and 1 mg of BC2 is added to 200 μL of Ti treated proteasomal sample. 2.5 μL of TCEP (100 mg/mL) and 2 μL of Omniligase is added to each of the sample. The reaction is incubated for 1 h at 37° C. and quenched with 25 μL of 4% TFA in Acetonitrile/Water (1:1 v/v) solution. The result are barcoded samples comprising proteins modified with peptide-barcodes at their N-termini.

Mixing and ArgC digestion of barcoded-samples: The samples are mixed together and digested using ArgC protease. Briefly, 1 μg of argC protease (Promega) are added to the pooled protein mixture and incubated for 16 h at 37° C. 2 mM DTT is added to activate the enzyme. The reaction is stopped by adding 10% formic acid.

Sample preparation for fluorosequencing: Peptides generated following argC digestion contain a C-terminal Arginine residue. C-termini differentiation, lysine and acidic residue labeling (as described in section—Selective Amino Acid Labeling, scheme 2 and scheme 3) are performed. The two amino-acids lysine and acid residues (glutamic acid, aspartic acid) are labeled with fluorophores Atto647N (Atto-tec) and Janelia Fluor (JF549, Tocris), respectively. The result are fluorescently labeled peptides, where the C-termini are labeled with an alkyne moiety, and the lysines and acidic residues labeled with fluorophores.

Fluorosequencing: For single-molecule peptide sequencing, a 40 mm German Desag 263 borosilicate glass coverslip (Bioptechs) surfaces are first cleaned by UV/ozone and then functionalized by soaking the coverslips for 30 min in methanol containing 0.01% azidopropyltriethoxysilane (Gelest) and 4 mM acetic acid. Weakly attached silane is removed by agitating the coverslips gently for 10 min in a bath of methanol, and subsequently gently agitating the coverslips for 10 min in water. The coverslip with immobilized peptides is dried under a nitrogen gas stream and baked in a vacuum oven for 20 min at 110° C. Peptides are covalently coupled to the coverslip surface via copper-catalyzed click chemistry between the alkyne-modified C-terminal AA residue and the azido silane. Coverslips are incubated with a fresh solution of 2 mM copper sulfate, 1 mM tris(3-hydroxypropyltriazolylmethyl)amine (Sigma), 20 mM HEPES (pH 8.0), and 5 mM sodium ascorbate with fluorescently labeled angiotensin for 30 min at room temperature. The coverslips are then washed with water to remove unbound peptides and dried under a nitrogen gas stream. Single-molecule sequencing is performed as described. Fluorosequencing datasets are analyzed using SigProc software tool. The raw image files are uploaded to Zenodo.

Data analysis and protein quantification: The unique target peptides containing the barcode peptides for PSME4 and PSMB10 are named MEPAER (SEQ ID NO: 11) and MLKPALEPR (SEQ ID NO: 12), respectively. Through the two different samples, the peptides are associated with the sample specific barcodes—BC1 and BC2. By collating the counts of BC1-PSME4, BC1-PSMB10 and BC2-PSME4, BC2-PSMB10 in the same fluorosequencing experiment, ratiometric measurements of the PSME4/PSMB10 in the two samples are made, which produce quantitative results to determine the proteasomal makeup between the control and the interferon activated cell line.

### Example 2: Data Storage and Retrieval Using Peptide Barcode Systems

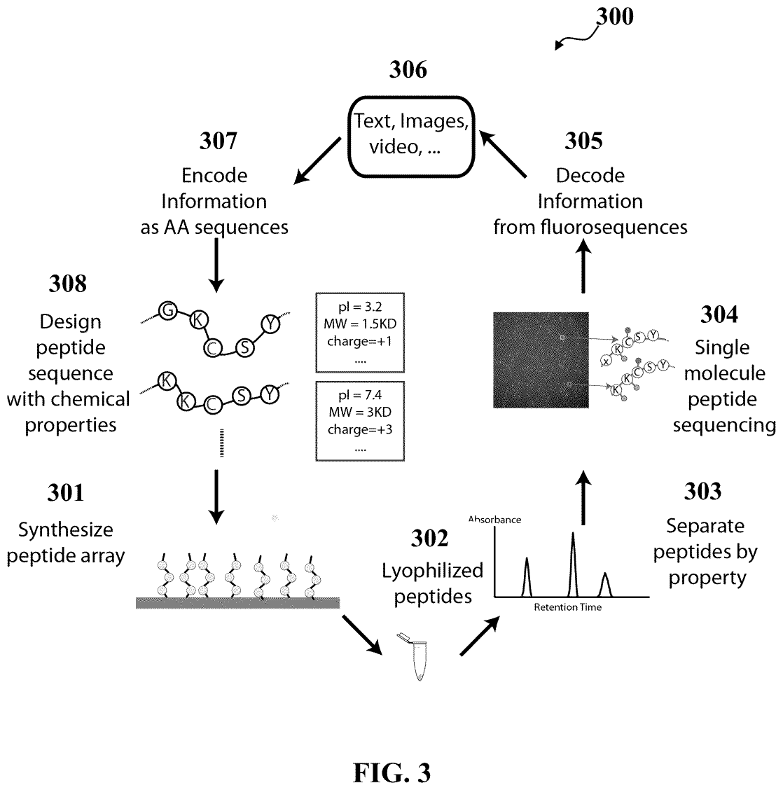

FIG. 3 illustrates a method for storing and/or retrieving information from a peptide barcode system 300. A plurality of peptide barcodes are synthesized and coupled to an array (301). The array is treated, for example, lyophilized (302) or formaldehyde-fixed, to enhance peptide barcode stability. The peptide barcodes are separated or differentially activated or identified for analysis by isoelectric points (303). Information is retrieved from the array through peptide barcode analysis. All array peptide barcodes are analyzed simultaneously, or peptide barcodes are analyzed separately based on distinct peptide barcode groups. The information retrieval is spatially resolved, such that a single peptide of the array provides position-specific information, e.g., by fluorosequencing (304). The information, e.g., fluorosequencing information, is decoded (305) and/or converted into other forms of information, such as text, images, and/or videos (306). The information is then be encoded as an amino acid (AA) sequence of the barcodes (307) for data storage. Such information is optionally re-recorded into new peptide arrays for further storage, and/or design and analysis (308).

For single molecule Edman sequencing, a #1 (1.7 mm) glass cover slip surface is cleaned by UV/ozone and functionalized by amino-silanization with aminopropltriethyoxysilane (APTES). Slide surfaces are further passivated by overnight incubation with polyethylene glycol (PEG)-NHS solution, prepared by dissolving a mixture of 80 mg of mPEG-SVA and 4 mg tboc-PEG-SVA in a sodium bicarbonate solution (pH 8.2). Functionalized slides are stored in a vacuum desiccator until use. The t-butyloxycarbonyl protecting groups are removed by incubating a slide with 90% TVA (v/v in water) for 5 h before use, exposing free amine groups for peptide immobilization. To aid in surface passivation, PEG slides are optionally treated with a 2% solution of Tween 20 in TRIS for 30 minutes.

Peptides are covalently coupled to the cover slip surface via amide bonds between the carboxylic acid of the C-terminal amino acid residue and the glass surface amines. Fresh solutions of 4 mM of 1-ethyl-3-(3-dimethylamino)propyl carbodiimide, hydrochloride (EDC) and 10 mM of N-hydroxysuccinimide (NHS) is made in 0.1 M MES buffer in 0.1 M 2-(N-morpholino)ethanesulfonic acid (MES) just before use. A solution of fluorescently labeled peptide (200 μM) is diluted with EDC-NHS solution (1:1 mixture by volume) to a final concentration of 20 μM peptide, 1.6 mM EDC, and 4 mM NHS. The mixture is stirred for 4 hr at room temperature before preparing an initial dilution series in 0.1M MES. Peptides are titrated from a secondary dilution series to between 20 μM and 2 nM peptide in 0.1 M NaHCO₃ to provide an attachment density on the slide of approximately 10 molecules per square nanometer. Peptides are incubated on the slide for 20 minutes before washing with water and methanol to remove unbound peptide. 1 μm-long 12-mercaptododecanoic acid NHS ester-functionalized gold nanorods are covalently attached to the slide via the amines to serve as fiducial markers for focusing and imaging registration.

After attaching the peptides and nanorods, the slide is incubated in 90% TFA (v/v in water) for 5 h then rinsed with methanol to remove boc group sand expose the peptides' free amino termini. Alternatively, to remove fmoc protecting groups, peptides are incubated for 1 h in 20% piperidine solution (in DMF), then washed with DMF and methanol to remove residual piperidine. An optional 1 hour incubation with 1,8-diazabicyclo[5.4.0]undec-7-ene (DBU) is used to remove peptides non-specifically bound to the surface of the slide. To assist with focus stability, the chamber and microscope are equilibrated at 40° C. during de-blocking and up to an additional 12 h.

Single molecule total internal reflection (TIRF) microscopy experiments are performed with a Nikon Ti-E inverted microscope equipped with a CFI Apo 60X/1.49NA oil immersion objective lens, a motorized stage with 100 nm resolution linear encoder, an iXon3 DU-897E 512×512 EMCCD detector operated at −70° C., and a MLC400B laser combiner with 561 nm and 647 nm lasers. Fluorescence from Atto647N is excited using 6.0 mW or 2.8 mW of 647 nm laser power via 647 LP dichoric and collected through 665LP and 705/72BP emission filters. Fluorescence for tetramethylrhodamine (TMR) is excited sing 2.7 mW of 561 nm laser power via 561LP dichroic and collected through 575LP and 600/50BP emission filters. Gold nanorod reflection is excited using <0.01 mW of 561 nm laser light using a 95/5 reflectance cube. To increase the number of pixels in an individual diffraction limited spot and to maximize the flat-field portion of the image collected, an additional 1.5× tube lens is inserted into the beam path.

Automated Edman degradation: For single-molecule Edman sequencing experiments, the sample temperature is maintained at 40° C. by heating both the perfusion chamber and microscope objective. Edman reagents are bubbled with dry nitrogen gas for 10 min, and then kept under nitrogen gas throughout the experiment. Solvent exchanges in the fluidic device are controlled using Python scripts and coordinated with image acquisition via custom macros in the Nikon Elements software package. Reagents are introduced to the perfusion chamber as shown in TABLE 4.

TABLE 4

| | | | Incubation | Incubation |
|---|---|---|---|---|
| | | | time (min) | time (min) |
| Protocol Step | | Reagents | System A | System B |
| Step | Pump wash | Water | wash | wash |
| 1 | | | | |
| Step | Methanol wash | Methanol | wash | wash |
| 2 | | | | |
| Step | Free basing solution | 1. 10:3:2:1 v/v acetonitrile, | wash | 5 |
| 3 | | pyridine, triethylamine, water | | |
| | | 2. 2:1 v/v acetonitrile, | | |
| | | triethylamine | | |
| Step | Mock or Edman | 100% acetonitrile or 9:1 v/v | 30 | 20 |
| 4 | solution | acetonitrile:phenylisothiocyanate | | |
| Step | Free basing solution | 1. 10:3:2:1 v/v acetonitrile, | wash | NA |
| 5 | | pyridine, triethylamine, water | | |
| | | 2. 2:1 v/v acetonitrile, | | |
| | | triethylamine | | |
| Step | Ethylacetate/ACN | Ethyl acetate or acetonitrile | wash | wash |
| 6 | wash | | | |
| Step | Cleavage solution | 100% trifluoroacetic acid | 30 | 15 |
| 7 | | | | |
| Step | Ethylacetate wash | Ethyl acetate | wash | wash |
| 8 | | | | |
| Step | Pump wash | Water | wash | wash |
| 9 | | | | |
| Step | Methanol wash | Methanol | wash | wash |
| 10 | | | | |
| Step | Oxygen scavenging | Trolox in methanol (1 mM) | 10 | 5.5 |
| 11 | solution | | | |

“Wash” denotes exchanging the solvents in the flow chamber (approx. 3 minutes). To distinguish signal loss due specifically to Edman chemistry, as many as four mock Edman cycles using all reagents except PITC are performed prior to Edman cycles. In total, steps 1-11 take approx. 1 to 1.5 hours.

Images are processed using each field view taken after each consecutive Edman cycle. Data are processed to measure changes in single molecule dye fluorescence intensities. The image processing and intensity measurements involve the following steps—(a) collating of TIRF images through multiple Edman cycles and fluorescent channels, (b) identifying fluorescent peptides after background filtering and fitting a point spread function around every individual fluorescent spot—termed peak, (c) extracting intensity values by summing the pixel values under the peak and (d) creating arrays of intensity values for every single fluorescent spot through channels and cycles. The multidimensional array includes intensities for every individual peak through each Edman cycle and fluorescent channel. A model of intensity is pre-determined for every individual dye, which maps to a unique log-normal distribution with a median intensity and spread, resulting in an estimate of the dye count based on the intensity value alone. The change in intensity for each dye is monitored as a removal of the select labeled amino acid. The result of the computation is the determination of a “dye-track”, where the counts of the different dyes for every individual fluorescent peptide molecule after every Edman cycle is produced. Simultaneously, mapping the reference database to the expected dye-tracks for proteins and peptides with the inclusion of the physico-chemical errors (a Monte-Carlo process) is simulated. The mapping of the dye-tracks obtained from the Edman sequencing experiment is matched to the results of the simulation. The entire process produces a list of peptides identified in the mixture with an assigned probability.

While preferred embodiments of the present invention have been shown and/or described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

The following non-limiting embodiments provide illustrative examples of the invention, but do not limit the scope of the invention.

Embodiment 1. A method for identifying a biomolecule, the method comprising:
(a) providing the biomolecule having coupled thereto an oligomeric barcode, wherein the oligomeric barcode comprises a plurality of monomeric subunits, wherein at least a subset of the monomeric subunits comprise a label; and
(b) identifying the label, wherein the identifying is by sequencing by degradation.

Embodiment 2. The method of embodiment 1, wherein the biomolecule is a polypeptide.

Embodiment 3. The method of embodiment 1, wherein the biomolecule is a protein.

Embodiment 4. The method of any one of embodiments 1-3, further comprising coupling the oligomeric barcode to the biomolecule.

Embodiment 5. The method of embodiment 4, wherein the coupling comprises enzymatic ligation.

Embodiment 6. The method of embodiment 4, wherein the coupling comprises transesterification.

Embodiment 7. The method of embodiment 4, wherein the coupling comprises chemical coupling or enzymatic coupling.

Embodiment 8. The method of embodiment 4, wherein the coupling comprises expressing the biomolecule coupled to the oligomeric barcode or co-translation of the oligomeric barcode as a peptide tag.

Embodiment 9. The method of embodiment 4, wherein the coupling comprises expressing the biomolecule coupled to the oligomeric barcode.

Embodiment 10. The method of embodiment 4, wherein the coupling comprises chemically synthesizing the biomolecule having coupled thereto the oligomeric barcode.

Embodiment 11. The method of any one of embodiments 1-10, wherein the oligomeric barcode comprises a polymer.

Embodiment 12. The method of any one of embodiments 1-11, wherein the oligomeric barcode comprises a polypeptide.

Embodiment 13. The method of any one of embodiments 1-12, wherein the oligomeric barcode comprises from about 2 to about 30 amino acids.

Embodiment 14. The method of any one of embodiments 1-13, wherein the oligomeric barcode comprises a non-natural amino acid.

Embodiment 15. The method of any one of embodiments 1-14, wherein the plurality of monomeric subunits is a plurality of amino acids.

Embodiment 16. The method of any one of embodiments 1-15, wherein the label is coupled to an internal monomeric subunit of the plurality of monomeric subunits.

Embodiment 17. The method of any one of embodiments 1-16, wherein the label is an amino acid specific label.

Embodiment 18. The method of embodiment 17, wherein the amino acid specific label comprises a methionine specific label, an arginine specific label, a histidine specific label, a tyrosine specific label, a carboxylic acid R-group specific label, a lysine specific label, a cysteine specific label, a tryptophan specific label, or any combination thereof.

Embodiment 19. The method of embodiment 17, wherein the amino acid specific label comprises a non-natural amino acid specific label.

Embodiment 20. The method of embodiment 19, wherein the non-natural amino acid specific label is a phosphoserine specific label, phosphothreonine specific label, pyroglutamic acid specific label, hydroxyproline specific label, azidolysine specific label, or dehydroalanine specific label.

Embodiment 21. The method of any one of embodiments 1-20, wherein the label is a fluorescent label.

Embodiment 22. The method of any one of embodiments 1-21, wherein the label is a dye.

Embodiment 23. The method of any one of embodiments 1-22, wherein the sequencing by degradation comprises Edman degradation.

Embodiment 24. The method of any one of embodiments 1-22, wherein the sequencing by degradation comprises subjecting the oligomeric barcode to conditions sufficient to remove at least one monomeric subunit from the oligomeric barcode.

Embodiment 25. The method of any one of embodiments 1-22, wherein the sequencing by degradation comprises subjecting the oligomeric barcode to conditions sufficient to remove at least one amino acid from the oligomeric barcode.

Embodiment 26. The method of any one of embodiments 1-25, wherein the label generates at least one signal or at least one signal change.

Embodiment 27. The method of embodiment 26, wherein the at least one signal or the at least one signal change is an optical signal.

Embodiment 28. The method of embodiment 26, wherein the at least one signal or the at least one signal change comprises a plurality of signals of different intensities.

Embodiment 29. The method of embodiment 26, wherein the at least one signal or the at least one signal change comprises a plurality of signals of different frequencies or signals of different frequency ranges.

Embodiment 30. The method of any one of embodiments 1-29, wherein the sequencing by degradation comprises enzymatic cleavage of the oligomeric barcode from the biomolecule.

Embodiment 31. The method of any one of embodiments 1-29, wherein the sequencing by degradation comprises chemical cleavage of the oligomeric barcode from the biomolecule.

Embodiment 32. The method of embodiment 31, wherein the chemical cleavage comprises cyanogen bromide cleavage, BNPS-skatole cleavage, formic acid cleavage, hydroxylamine cleavage, 2-nitro-5-thiocyanobenzoic acid cleavage, or any combination thereof.

Embodiment 33. The method of any one of embodiments 1-32, wherein the oligomeric barcode is coupled to the biomolecule via an N-terminal tag, a C-terminal tag, or an amino acid sidechain.

Embodiment 34. The method of embodiment 33, wherein the N-terminal tag is a purification tag, a localization signal, a fluorescent tag, a chemically modifiable tag, or an enzymatically modifiable tag.

Embodiment 35. The method of embodiment 33, wherein the C-terminal tag is a purification tag, a localization signal, a fluorescent tag, a chemically modifiable tag, or an enzymatically modifiable tag.

Embodiment 36. The method of any one of embodiments 1-35, wherein the oligomeric barcode is coupled to the biomolecule via a cleavable linker.

Embodiment 37. The method of embodiment 36, wherein the cleavable linker comprises a TEV protease cleavage site, a thrombin cleavage site, an enterokinase cleavage site, or any combination thereof.

Embodiment 38. The method of embodiment 36, wherein the cleavable linker comprises an amino acid cleavage sequence not present in the oligomeric barcode.

Embodiment 39. The method of embodiment 36, wherein the cleavable linker comprises a chemically cleavable group.

Embodiment 40. The method of embodiment 39, wherein the chemically cleavable comprises a disulfide.

Embodiment 41. The method of embodiment 36, further comprising cleaving the oligomeric barcode from the biomolecule.

Embodiment 42. The method of embodiment 41, further comprising separating the oligomeric barcode from the biomolecule after the cleaving.

Embodiment 43. The method of embodiment 42, wherein the separating comprises isoelectric focusing.

Embodiment 44. The method of embodiment 42, wherein the separating comprises chromatographic separation.

Embodiment 45. The method of embodiment 42, wherein the separating comprises electrophoretic separation.

Embodiment 46. The method of any one of embodiments 41-45, further comprising coupling the oligomeric barcode to a substrate after the cleaving.

Embodiment 47. The method of any one of embodiments 41-45, further comprising coupling the oligomeric barcode to a substrate after the separating.

Embodiment 48. The method of any one of embodiments 1-47, wherein the oligomeric barcode is selected from a library comprising at least 216 uniquely identifiable oligomeric barcodes.

Embodiment 49. The method of any one of embodiments 1-48, wherein the identifying comprises a resolution capable of resolving a single oligomeric barcode.

Embodiment 50. The method of any one of embodiments 1-49, wherein the biomolecule and the oligomeric barcode comprise a common sequence.

Embodiment 51. A method comprising:
(a) providing a polypeptide immobilized to a support, wherein the polypeptide comprises at least one labeled internal amino acid, and wherein the polypeptide encodes data;
(b) detecting at least one signal or signal change from the polypeptide immobilized to the support to identify at least a portion of a sequence of the polypeptide; and
(c) subjecting the polypeptide to conditions sufficient to remove at least one amino acid from the polypeptide.

Embodiment 52. The method of embodiment 51, wherein the at least one labeled internal amino acid comprises a plurality of amino acid specific labels.

Embodiment 53. The method of embodiment 52, wherein the amino acid specific labels comprise a methionine specific label, an arginine specific label, a histidine specific label, a tyrosine specific label, a carboxylic acid-containing amino acid specific label, a lysine specific label, a cysteine specific label, or any combination thereof.

Embodiment 54. The method of any one of embodiments 51-53, wherein the at least one labeled internal amino acid comprises an optically detectable label.

Embodiment 55. The method of any one of embodiments 51-54, wherein the at least one amino acid is removed from an N-terminus of the polypeptide.

Embodiment 56. The method of any one of embodiments 51-55, wherein, subsequent to (c), the at least one labeled internal amino acid becomes a labeled terminal amino acid.

Embodiment 57. The method of any one of embodiments 51-56, wherein the at least one labeled internal amino acid is from a plurality of labeled amino acids, and wherein the at least one signal or signal change comprises a collective signal from the plurality of labeled amino acids.

Embodiment 58. The method of embodiment 57, wherein the plurality of labeled amino acids comprise amino acids with different labels.

Embodiment 59. The method of embodiment 58, wherein the different labels generate signals with different signal patterns.

Embodiment 60. The method of any one of embodiments 51-59, wherein the at least one labeled internal amino acid comprises one or more members selected from the group consisting of lysine, glutamate, and aspartate.

Embodiment 61. The method of any one of embodiments 51-60, wherein the at least one labeled internal amino acid comprises an amino acid having a dye coupled thereto, which dye generates the at least one signal or signal change.

Embodiment 62. The method of any one of embodiments 51-61, wherein the at least one signal or signal change is an optical signal.

Embodiment 63. The method of any one of embodiments 51-62, wherein the at least one signal or signal change comprises a plurality of signals of different intensities.

Embodiment 64. The method of any one of embodiments 51-63, wherein the at least one signal or signal change comprises a plurality of signals of different frequencies or frequency ranges.

Embodiment 65. The method of any one of embodiments 51-64, further comprising cleaving the polypeptide from the support.

Embodiment 66. The method of any one of embodiments 51-65, wherein at least one amino acid is removed from the polypeptide by a degradation reaction.

Embodiment 67. The method of embodiment 66, wherein the degradation reaction is Edman degradation.

Embodiment 68. The method of any one of embodiments 51-67, wherein the polypeptide is a protein.

Embodiment 69. The method of any one of embodiments 51-67, wherein the polypeptide is part of a protein.

Embodiment 70. The method of any one of embodiments 51-69, wherein the at least one signal or signal change is detected with an optical detector having single-molecule sensitivity.

Embodiment 71. The method of any one of embodiments 51-70, further comprising processing the at least the portion of the sequence against a reference sequence to identify the polypeptide or a protein from which the polypeptide is derived.

Embodiment 72. The method of any one of embodiments 51-71, further comprising, subsequent to (c), (i) identifying the at least the portion of the sequence of the polypeptide to identify the polypeptide, and (ii) using the polypeptide identified in (i) to quantify the polypeptide or a protein from which the polypeptide was derived.

Embodiment 73. The method of any one of embodiments 51-72, wherein in (a), less than all amino acids of the polypeptide are labeled.

Embodiment 74. The method of any one of embodiments 51-73, further comprising (i) repeating (b) and (c) to detect at least one additional signal or signal change from the polypeptide immobilized to the support and (ii) using the at least one signal or signal change and the at least one additional signal or signal change to identify the at least the portion of the sequence.

Embodiment 75. The method of any one of embodiments 51-74, wherein the detecting identifies a sequence of the polypeptide.

Embodiment 76. The method of any one of embodiments 51-75, wherein the detecting is performed at a read rate of at least 36 bits/s.

Embodiment 77. The method of any one of embodiments 51-76, wherein the detecting comprises fluorimetry.

Embodiment 78. The method of any one of embodiments 51-77, wherein the detecting comprises imaging.

Embodiment 79. The method of any one of embodiments 51-78, further comprising assigning the polypeptide a optically resolvable address.

Embodiment 80. The method of embodiment 80, wherein the optically resolvable address comprises digital information.

Embodiment 81. The method of any one of embodiments 51-80, further comprising comparing the portion of the sequence of the polypeptide against a database of known sequences.

Embodiment 82. The method of any one of embodiments 51-81, further comprising, prior to (a), coupling the polypeptide to the support.

Embodiment 83. The method of any one of embodiments 51-82, further comprising determining a physical property of the polypeptide.

Embodiment 84. The method of embodiment 83, wherein the physical property is selected from the group consisting of isoelectric point, molecular weight, and hydrophobicity index.

Embodiment 85. The method of any one of embodiments 51-84, further comprising, prior to (a), coupling the polypeptide to an array.

Embodiment 86. The method of embodiment 85, further comprising lyophilizing the array.

Embodiment 87. The method of embodiment 86, wherein the array comprises an information storage density of at least 10⁷ bytes/cm³.

Embodiment 88. The method of embodiment 86, wherein the array comprises an information storage density of at least 10³⁰ bytes/cm³.

Embodiment 88a. The method of any one of embodiments 51-88, wherein the data are text.

Embodiment 88b. The method of any one of embodiments 51-88, wherein the data are an image.

Embodiment 88c. The method of any one of embodiments 51-88, wherein the data are numerical data.

Embodiment 88d. The method of any one of embodiments 51-88, wherein the data are multimedia.

Embodiment 89. A method comprising:
(a) providing a plurality of vectors, wherein each of the plurality of vectors comprises a first nucleotide sequence encoding a polypeptide and a second nucleotide sequence encoding a peptide barcode;
(b) transforming the plurality of vectors to produce a plurality of polypeptides, wherein the polypeptide barcode is coupled to a polypeptide from the plurality of polypeptides;
(c) selecting the polypeptide based on a condition; and
(d) identifying the peptide barcode coupled to the polypeptide from the plurality of polypeptides, wherein the identifying is by sequencing by degradation.

Embodiment 90. The method of embodiment 89, wherein the peptide is a protein.

Embodiment 91. The method of any one of embodiments 89-90, wherein each of the plurality of vectors comprises a plasmid, a phagemid, a cosmid, fosmid, or any combination thereof.

Embodiment 92. The method of any one of embodiments 89-91, wherein each of the plurality of vectors further comprises a sequence encoding an enrichment tag.

Embodiment 93. The method of any one of embodiments 89-92, wherein each of the plurality of vectors further comprises a sequence encoding a cleavage tag, and wherein the cleavage tag is positioned between the first nucleotide sequence and the second nucleotide sequence.

Embodiment 94. The method of any one of embodiments 89-93, wherein each of the plurality of vectors comprises a promoter upstream of the first nucleotide sequence.

Embodiment 95. The method of any one of embodiments 89-94, wherein each of the plurality of vectors comprises a selection marker.

Embodiment 96. The method of any one of embodiments 89-95, wherein the transforming comprises transient transfection, stable transfection, DEAE-dextran-mediated transfection, electroporation, liposome-mediated transfection, calcium phosphate co-precipitation, calcium chloride co-precipitation, microinjection, or any combination thereof.

Embodiment 97. The method of any one of embodiments 89-96, wherein the transforming comprises introducing the first nucleotide sequence and the second nucleotide sequence into a host organism genome.

Embodiment 98. The method of embodiment 97, wherein the introducing comprises CRISPR-Cas enzymatic cleavage, homologous recombination, or any combination thereof.

Embodiment 99. The method of any one of embodiments 89-98, wherein the peptide comprises an antibody.

Embodiment 100. The method of embodiment 99, wherein the antibody comprises an IgA antibody, an IgD antibody, an IgE antibody, an IgG antibody, an IgM antibody, an IgW antibody, an IgY antibody, an IgNAR antibody, an hcIgG antibody, a camel Ig antibody, a minibody, a nanobody, a single domain antibody, a diabody, a triabody, or any combination thereof.

Embodiment 101. The method of any one of embodiments 89-100, further comprising cleaving the peptide barcode from the polypeptide.

Embodiment 102. The method of any one of embodiments 89-101, wherein the polypeptide barcode comprises a label.

Embodiment 103. The method of any one of embodiments 89-102, wherein the peptide barcode comprises a plurality of labels.

Embodiment 104. The method of embodiment 103, wherein the plurality of labels comprises an amino acid specific label.

Embodiment 105. The method of embodiment 103 or 104, wherein the plurality of labels comprises a methionine specific label, an arginine specific label, a histidine specific label, a tyrosine specific label, a carboxylic acid R-group specific label, a lysine specific label, a cysteine specific label, a tryptophan specific label, or any combination thereof.

Embodiment 106. The method of embodiment 103, wherein the plurality of labels comprises a non-natural amino acid specific label.

Embodiment 107. The method of embodiment 106, wherein the non-natural amino acid specific label is a phosphoserine specific label, phosphothreonine specific label, pyroglutamic acid specific label, hydroxyproline specific label, azidolysine specific label, or dehydroalanine specific label.

Embodiment 108. The method of embodiment 102, wherein the label is a fluorescent label.

Embodiment 109. The method of embodiment 102, wherein the label is a dye.

Embodiment 110. The method of any one of embodiments 89-109, wherein the sequencing by degradation comprises Edman degradation.

Embodiment 111. The method of any one of embodiments 89-110, wherein the sequencing by degradation comprises subjecting the peptide barcode to conditions sufficient to remove at least one amino acid from the peptide barcode.

Embodiment 112. The method of embodiment 102, wherein the label generates at least one signal or at least one signal change.

Embodiment 113. The method of embodiment 112, wherein the at least one signal or the at least one signal change is an optical signal.

Embodiment 114. The method of embodiment 112, wherein the at least one signal or the at least one signal change comprises a plurality of signals of different intensities.

Embodiment 115. The method of embodiment 112, wherein the at least one signal or the at least one signal change comprises a plurality of signals of different frequencies or signals of different frequency ranges.

Embodiment 116. The method of any one of embodiments 89-115, wherein the sequencing by degradation comprises cleaving the peptide barcode from the polypeptide.

Embodiment 117. The method of embodiment 116, further comprising separating the peptide barcode from the biomolecule after the cleaving.

Embodiment 118. The method of embodiment 117, wherein the separating comprises isoelectric focusing.

Embodiment 119. The method of embodiment 117, wherein the separating comprises chromatographic separation.

Embodiment 120. The method of embodiment 117, wherein the separating comprises electrophoretic separation.

Embodiment 121. The method of embodiment 116, wherein the sequencing comprises enzymatic or chemical cleavage of the peptide barcode.

Embodiment 122. The method of embodiment 116, further comprising coupling the peptide barcode to a substrate after the cleaving.

Embodiment 123. The method of any one of embodiments 117-121, further comprising coupling the peptide barcode to a substrate after the separating.

Embodiment 124. The method of any one of embodiments 89-123, wherein the identifying comprises detecting at a resolution capable of resolving a single peptide barcode.

Embodiment 125. A plasmid encoding a polypeptide coupled to an oligopeptide barcode, the plasmid comprising an open reading frame downstream from a promoter, wherein the open reading frame comprises a sequence encoding the polypeptide and a sequence encoding the oligopeptide barcode, and wherein the oligopeptide barcode comprises a sequence that uniquely identifies the polypeptide.

Embodiment 126. The plasmid of embodiment 125, wherein the open reading frame further comprises a sequence encoding a cleavage site.

Embodiment 127. The plasmid of embodiment 125 or 126, wherein the sequence encoding the cleavage site is positioned between the sequence encoding the polypeptide and the sequence encoding the oligomeric peptide.

Embodiment 128. The plasmid of any one of embodiments 125-127, wherein the sequence encoding cleavage site comprises a protease recognition sequence, and wherein the protease recognition sequence is not present in the sequence encoding the polypeptide.

Embodiment 129. The plasmid of embodiment 128, wherein the protease recognition sequence comprises a TEV protease recognition sequence, a thrombin recognition sequence, an enterokinase recognition sequence, or any combination thereof.

Embodiment 130. The plasmid of any one of embodiments 125-129, wherein the open reading frame further comprises a sequence encoding an enrichment tag.

Embodiment 131. The plasmid of embodiment 130, wherein the sequence encoding the enrichment tag is positioned between the sequence encoding the polynucleotide and the sequence encoding the oligomeric peptide.

Embodiment 132. The plasmid of any one of embodiments 125-131, further comprising a selection marker.

Embodiment 133. The plasmid of any one of embodiments 125-132, further comprising a promoter upstream of the open reading frame.

Embodiment 134. The plasmid of any one of embodiments 125-133, wherein the promoter is a constitutive promoter.

1. A method for identifying a biomolecule, said method comprising:
(a) providing said biomolecule having coupled thereto an oligopeptide barcode associated with the biomolecule's identity,
wherein said oligopeptide barcode comprises a plurality of amino acid residues, wherein at least a subset of said amino acid residues comprise a label; and
(b) identifying said label(s) by sequencing by degradation, thereby identifying the oligopeptide and the biomolecule coupled thereto.

2. The method of claim 1, wherein said biomolecule is a polypeptide.

3-7. (canceled)

8. The method of claim 1, wherein said biomolecule is coupled to said oligopeptide barcode by co-expression or co-translation of said oligopeptide barcode as a peptide tag.

9. (canceled)

10. The method of claim 1, wherein said biomolecule is coupled to said oligopeptide by chemical synthesis.

11-12. (canceled)

13. The method of claim 1, wherein said oligopeptide barcode comprises from about 2 to about 30 amino acids.

14. The method of claim 1, wherein said oligopeptide barcode comprises a non-natural amino acid.

15. (canceled)

16. The method of claim 1, wherein said label is coupled to an internal amino acid residue of said plurality of amino acid residues.

17. The method of claim 1, wherein said label is an amino acid specific label.

18. The method of claim 17, wherein said amino acid specific label comprises a methionine specific label, an arginine specific label, a histidine specific label, a tyrosine specific label, a carboxylic acid R-group specific label, a lysine specific label, a cysteine specific label, a tryptophan specific label, or any combination thereof.

19. The method of claim 17, wherein said amino acid specific label comprises a non-natural amino acid specific label.

20. The method of claim 19, wherein said non-natural amino acid specific label is a phosphoserine specific label, phosphothreonine specific label, pyroglutamic acid specific label, hydroxyproline specific label, azidolysine specific label, or dehydroalanine specific label.

21-25. (canceled)

26. The method of claim 1, wherein said label generates at least one signal or at least one signal change.

27. The method of claim 26, wherein said at least one signal or said at least one signal change is an optical signal.

28. The method of claim 26, wherein said at least one signal or said at least one signal change comprises a plurality of signals of different intensities.

29. (canceled)

30. The method of claim 1, wherein said sequencing by degradation comprises enzymatic cleavage of said oligopeptide barcode from said biomolecule.

31. The method of claim 1, wherein said sequencing by degradation comprises chemical cleavage of said oligopeptide barcode from said biomolecule.

32. (canceled)

33. The method of claim 1, wherein said oligopeptide barcode is coupled to said biomolecule via an N-terminal tag, a C-terminal tag, or an amino acid sidechain.

34-50. (canceled)

51. A method comprising:
(a) providing a polypeptide immobilized to a support, wherein said polypeptide comprises at least one labeled internal amino acid, and wherein said polypeptide encodes data;
(b) detecting at least one signal or signal change from said polypeptide immobilized to said support to identify at least a portion of a sequence of said polypeptide; or
(c) subjecting said polypeptide to conditions sufficient to remove at least one amino acid from said polypeptide.

52-91. (canceled)
